SEQUENCE LISTING (15 sequences)

SEQ ID NO: 1 - DNA, 34 bp, Artificial Sequence
  Primer
gtcgtatgagctctgctgaaactgttgaaagttg

SEQ ID NO: 2 - DNA, 21 bp, Artificial Sequence
  Primer
ctgaacaccctgaacaaagtc

SEQ ID NO: 3 - DNA, 22 bp, Artificial Sequence
  Primer
cgaattctcagatggcccaggt

SEQ ID NO: 4 - DNA, 22 bp, Artificial Sequence
  Primer
gaaaacgccgcggaaaagattg

SEQ ID NO: 5 - DNA, 8684 bp, Artificial Sequence
  Synthetic construct
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcacaagcgagctctgcttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgaccgcaacgcaattaatgtgagttagctcactcattaggcaccccaggctttacactttatgcttccggctcgtatgttgtgtggaattgtgagcggataacaatttcacccatgctttggacaggaaacagctatgaaaaagcttttattcgctatcccgttagttgtaccgttctattctcactctgccgagacagtcgaatcctgcctggccaaggtccacactgagaatagtttcacaaatgtgtggaaggatgataagacccttgatcgatatgccaattacgaaggctgcttatggaatgccaccggcgtcgttgtctgcacgggcgatgagacacaatgctatggcacgtgggtgccgataggcttagccataccggagaacgaaggcggcggtagcgaaggcggtggcagcgaaggcggtggatccgaaggaggtggaaccaagccgccggaatatggcgacactccgatacctggttacacctacattaatccgttagatggaacctaccctccgggcaccgaacagaatcctgccaacccgaacccaagcttagaagaaagccaaccgttaaacacctttatgttccaaaacaaccgttttaggaaccgtcaaggtgctcttaccgtgtacactggaaccgtcacccagggtaccgatcctgtcaagacctactatcaatataccccggtctcgagtaaggctatgtacgatgcctattggaatggcaagtttcgtgattgtgcctttcacagcggtttcaacgaagacccttttgtctgcgagtaccagggtcagagtagcgatttaccgcagccaccggttaacgcgggtggtggtagcggcggaggcagcggcggtggtagcgaaggcggaggtagcgaaggaggtggcagcggaggcggtagcggcagtggcgacttcgactacgagaaaatggctaatgccaacaaaggcgccatgactgagaacgctgacgagaatgcactgcaaagtgatgccaagggtaagttagacagcgtcgccacagactatggtgctgccatcgacggctttatcggcgatgtcagtggtctggctaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttctctgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 6 - DNA, 8108 bp, Artificial Sequence
  Synthetic construct
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcagatatcaacgatgatcgtatggctagcggcgccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgtttctgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 7 - DNA, 8684 bp, Artificial Sequence
  Synthetic construct
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcacaagcgagctctgcttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgaccgcaacgcaattaatgtgagttagctcactcattaggcaccccaggctttacactttatgcttccggctcgtatgttgtgtggaattgtgagcggataacaatttcacccatgctttggacaggaaacagctatgaaaaagcttttattcgctatcccgttagttgtaccgttctattctcactctgccgagacagtcgaatcctgcctggccaaggtccacactgagaatagtttcacaaatgtgtggaaggatgataagacccttgatcgatatgccaattacgaaggctgcttatggaatgccaccggcgtcgttgtctgcacgggcgatgagacacaatgctatggcacgtgggtgccgataggcttagccataccggagaacgaaggcggcggtagcgaaggcggtggcagcgaaggcggtggatccgaaggaggtggaaccaagccgccggaatatggcgacactccgatacctggttacacctacattaatccgttagatggaacctaccctccgggcaccgaacagaatcctgccaacccgaacccaagcttagaagaaagccaaccgttaaacacctttatgttccaaaacaaccgttttaggaaccgtcaaggtgctcttaccgtgtacactggaaccgtcacccagggtaccgatcctgtcaagacctactatcaatataccccggtctcgagtaaggctatgtacgatgcctattggaatggcaagtttcgtgattgtgcctttcacagcggtttcaacgaagacccttttgtctgcgagtaccagggtcagagtagcgatttaccgcagccaccggttaacgcgggtggtggtagcggcggaggcagcggcggtggtagcgaaggcggaggtagcgaaggaggtggcagcggaggcggtagcggcagtggcgacttcgactacgagaaaatggctaatgccaacaaaggcgccatgactgagaacgctgacgagaatgcactgcaaagtgatgccaagggtaagttagacagcgtcgccacagactatggtgctgccatcgacggctttatcggcgatgtcagtggtctggctaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttcggagccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 8 - DNA, 9023 bp, Artificial Sequence
  Synthetic construct
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctggcgcggccgaatcacatctagacggcgccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacagtgatagactagttagacgcgtgcttaaaggcctccaatcctcttggcgcgccaattctatttcaaggagacagtcataatgaaatacctattgcctacggcagccgctggattgttattactcgcggcccagccggccctctgataagatatcacttgtttaaactctgcttggccctcttggccttctagtagacttgcggccgcacatcatcatcaccatcacggggccgcagaacaaaaactcatctcagaagaggatctgaatggggccgcataggctagctctgctagtggcgacttcgactacgagaaaatggctaatgccaacaaaggcgccatgactgagaacgctgacgagaatgctttgcaaagcgatgccaagggtaagttagacagcgtcgcgaccgactatggcgccgccatcgacggctttatcggcgatgtcagtggtttggccaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttctctgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 9 - DNA, 5310 bp, Artificial Sequence
  Synthetic construct
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacaggtccaactgcaggtcgacctcgagatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgttaataaggcgcgccaattctatttcaaggagacagtcataatgaaatacctattgcctacggcagccgctggattgttattactcgcggcccagccggccatggcccaggtgcagctgcaggagagcggggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatataaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 10 - DNA, 9780 bp, Artificial Sequence
  Synthetic construct
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctggcgcggccgaatcacatctagacggcgccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgactgaatggcgaatggcgctttgcctggtttccggcaccagaagcggtgccggaaagctggctggagtgcgatcttcctgacgctcgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacagtgatagactagttagacgcgtgcttaaaggcctccaatcctcttggcgcgccaattctatttcaaggagacagtcataatgaaatacctattgcctacggcagccgctggattgttattactcgcggcccagccggccctctgataagatatcacttgtttaaactctgcttggccctcttggccttctagtagacttgcggccgcacatcatcatcaccatcacggggccgcagaacaaaaactcatctcagaagaggatctgaatggggccgcataggctagcgatatcaacgatgatcgtatggcttctactgccgagacagtcgaatcctgcctggccaagcctcacactgagaatagtttcacaaatgtgtggaaggatgataagacccttgatcgatatgccaattacgaaggctgcttatggaatgccaccggcgtcgttgtctgcacgggcgatgagacacaatgctatggcacgtgggtgccgataggcttagccataccggagaacgaaggcggcggtagcgaaggcggtggcagcgaaggcggtggatccgaaggaggtggaaccaagccgccggaatatggcgacactccgatacctggttacacctacattaatccgttagatggaacctaccctccgggcaccgaacagaatcctgccaacccgaacccaagcttagaagaaagccaaccgttaaacacctttatgttccaaaacaaccgttttaggaaccgtcaaggtgctcttaccgtgtacactggaaccgtcacccagggtaccgatcctgtcaagacctactatcaatataccccggtctcgagtaaggctatgtacgatgcctattggaatggcaagtttcgtgattgtgcctttcacagcggtttcaacgaagacccttttgtctgcgagtaccagggtcagagtagcgatttaccgcagccaccggttaacgcgggtggtggtagcggcggaggcagcggcggtggtagcgaaggcggaggtagcgaaggaggtggcagcggaggcggtagcggcagtggcgacttcgactacgagaaaatggctaatgccaacaaaggcgccatgactgagaacgctgacgagaatgcactgcaaagtgatgccaagggtaagttagacagcgtcgccacagactatggtgctgccatcgacggctttatcggcgatgtcagtggtctggctaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttctctgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 11 - DNA, 9413 bp, Artificial Sequence
  Synthetic construct
ttaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgaccgcaacgcaattaatgtgagttagctcactcattaggcaccccaggctttacactttatgcttccggctcgtatgttgtgtggaattgtgagcggataacaatttcacccatgctttggacaggaaacagctatgaaaaagcttttattcgctatcccgttagttgtaccgttctattctcactctgccgagacagtcgaatcctgcctggccaagtctcacactgagaatagtttcacaaatgtgtggaaggatgataagacccttgatcgatatgccaattacgaaggctgcttatggaatgccaccggcgtcgttgtctgcacgggcgatgagacacaatgctatggcacgtgggtgccgataggcttagccataccggagaacgaaggcggcggtagcgaaggcggtggcagcgaaggcggtggatccgaaggaggtggaaccaagccgccggaatatggcgacactccgatacctggttacacctacattaatccgttagatggaacctaccctccgggcaccgaacagaatcctgccaacccgaacccaagcttagaagaaagccaaccgttaaacacctttatgttccaaaacaaccgttttaggaaccgtcaaggtgctcttaccgtgtacactggaaccgtcacccagggtaccgatcctgtcaagacctactatcaatataccccggtctcgagtaaggctatgtacgatgcctattggaatggcaagtttcgtgattgtgcctttcacagcggtttcaacgaagacccttttgtctgcgagtaccagggtcagagtagcgatttaccgcagccaccggttaacgcgggtggtggtagcggcggaggcagcggcggtggtagcgaaggcggaggtagcgaaggaggtggcagcggaggcggtagcggcagtggcgacttcgactacgagaaaatggctaatgccaacaaaggcgccatgactgagaacgctgacgagaatgcactgcaaagtgatgccaagggtaagttagacagcgtcgccacagactatggtgctgccatcgacggctttatcggcgatgtcagtggtctggctaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttcggagccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcacaagcgagctctgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaa

SEQ ID NO: 12 - DNA, 9413 bp, Artificial Sequence
  Synthetic construct
ttaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgaccgcaacgcaattaatgtgagttagctcactcattaggcaccccaggctttacactttatgcttccggctcgtatgttgtgtggaattgtgagcggataacaatttcacccatgctttggacaggaaacagctatgaaaaagcttttattcgctatcccgttagttgtaccgttctattctcactctgccgagacagtcgaatcctgcctggccaagtctcacactgagaatagtttcacaaatgtgtggaaggatgataagacccttgatcgatatgccaattacgaaggctgcttatggaatgccaccggcgtcgttgtctgcacgggcgatgagacacaatgctatggcacgtgggtgccgataggcttagccataccggagaacgaaggcggcggtagcgaaggcggtggcagcgaaggcggtggatccgaaggaggtggaaccaagccgccggaatatggcgacactccgatacctggttacacctacattaatccgttagatggaacctaccctccgggcaccgaacagaatcctgccaacccgaacccaagcttagaagaaagccaaccgttaaacacctttatgttccaaaacaaccgttttaggaaccgtcaaggtgctcttaccgtgtacactggaaccgtcacccagggtaccgatcctgtcaagacctactatcaatataccccggtctcgagtaaggctatgtacgatgcctattggaatggcaagtttcgtgattgtgcctttcacagcggtttcaacgaagacccttttgtctgcgagtaccagggtcagagtagcgatttaccgcagccaccggttaacgcgggtggtggtagcggcggaggcagcggcggtggtagcgaaggcggaggtagcgaaggaggtggcagcggaggcggtagcggcagtggcgacttcgactacgagaaaatggctaatgccaacaaaggcgccatgactgagaacgctgacgagaatgcactgcaaagtgatgccaagggtaagttagacagcgtcgccacagactatggtgctgccatcgacggctttatcggcgatgtcagtggtctggctaacggcaacggagccaccggagacttcgcaggttcgaattctcagatggcccaggttggagatggggacaacagtccgcttatgaacaactttagacagtaccttccgtctcttccgcagagtgtcgagtgccgtccattcgttttctctgccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcacaagcgagctctgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaa

SEQ ID NO: 13 - DNA, 8492 bp, Artificial Sequence
  Synthetic construct
aattctcagatggcccaggttggagatggggacaacagtccgcttatgaacaactttagacagtaccttccgtctcttccgcagagtgtcgagtgccgtccattcgttttcggagccggcaagccttacgagttcagcatcgactgcgataagatcaatcttttccgcggcgttttcgctttcttgctatacgtcgctactttcatgtacgttttcagcactttcgccaatattttacgcaacaaagaaagctagtgatctcctaggaagcccgcctaatgagcgggctttttttttctggtatgcatcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcacagtgcacaatcacatctagacgcggccgctcatcaccaccatcatcactctgctgaacaaaaactcatctcagaagaggatctgaatggtgccgcacaagcgagctctgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggatccaagcttgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggcgcactagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgtacgtaagacccccaagcttgtcgacagtgatagactagttagacgcgtgcttaaaggcctccaatcctcttggcgcgccaattctatttcaaggagacagtcataatgaaatacctattgcctacggcagccgctggattgttattactcgcggcccagccggccctctgataagatatcacttgtttaaactctgcttggccctcttggccttctagtagacttg

SEQ ID NO: 14 - protein, 400 aa, Bacteriophage fd.
TVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILR

SEQ ID NO: 15 - protein, 400 aa, Bacteriophage fd
TVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILR

(19) United States
(12) Patent Application Publication
(10) Pub. No.: US 2004/0180422 A1
(43) Pub. Date: Sep. 16, 2004

(54) Methods and compositions for controlling valency of phage display

(75) Inventors: Rene Hoet, Maastricht (NL); Robert Charles Ladner, Ijamsville, MD (US); Nicolas Frans, Liege (BE)

Correspondence Address: FISH & RICHARDSON PC, 225 FRANKLIN ST, BOSTON, MA, 02110, US

(73) Assignee: DYAX CORP.

(21) Appl. No.: 10/723,981

(22) Filed: Nov. 26, 2003

Related U.S. Application Data
(60) Provisional application No. 60/429,134, filed on Nov. 26, 2002.

(51) Int. Cl. C12N 5/02; C12N 5/00; C12N 7/01; C12N 7/00; C07H 21/04; C12Q 1/70

(52) U.S. Cl. 435/235.1; 536/23.72; 536/23.5; 435/325

(57) ABSTRACT

Disclosed are methods and compositions useful, e.g., for controlling the valency of display proteins during display library screenings and selections. In one embodiment, they are applicable to phage and phage libraries that are based on bacteriophage, e.g., filamentous bacteriophage.

### CROSS-REFERENCE TO RELATED APPLICATIONS

This application claims the benefit of priority to U.S. Provisional Patent Application Serial No. 60/429,134, filed on Nov. 26, 2002, the entire contents of which are herein incorporated by reference.

### BACKGROUND

Phage display can be used to identify protein ligands that bind to a particular target. This technique uses bacteriophage particles as vehicles for linking candidate protein ligands to the nucleic acids encoding them. The coding nucleic acid is packaged within the bacteriophage, and the encoded protein can be expressed on the phage surface. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; WO 00/70023; US 2002-0102613; de Haard et al. (1999) J. Biol. Chem 274:18218-30; Hoogenboom et al. (1998) Immunotechnology 4:1-20; Hoogenboom et al. (2000) Immunol Today 2:371-8.

There are at least two general systems of phage display. In one system, the nucleic acid sequence encoding the display protein is included in the phage genome. In another system, this nucleic acid is located on a phagemid that is packaged in the phage particles. Co-infection of a host cell with helper phage (such as M13KO1) enables the phage particles to be produced that package phagemids. Particles that display a protein that binds to a particular target can be selected from the display library. The nucleic acid within the selected particles enables identification and isolation of the display protein.

### SUMMARY

The methods and compositions described herein are useful, e.g., for controlling the valency of proteins during display library screenings and selections. In particular, they are applicable to phage and phage libraries that are based on bacteriophage, e.g., filamentous bacteriophage.

In one aspect, the invention features a method that includes: providing a set of host cells. Each of the host cells of the set includes a) a first expression unit and b) second expression unit.

The first expression unit includes (1) a first open reading frame and (2) a first promoter operably linked to the first open reading frame. The first open reading frame encodes a first polypeptide including (i) an amino acid sequence to be displayed on a phage and (ii) a portion of a phage coat protein of a filamentous phage. The portion of the phage coat protein physically associates with phage particles.

The second expression unit includes (1′) a second open reading frame, encoding a second polypeptide including a portion of the phage coat protein, and (2′) a second promoter operably linked to the second open reading frame, wherein the second promoter is regulatable. The method can further include maintaining the set of host cells under a first condition, wherein phage particles that include amino acid sequences to be displayed are produced.

The amino acid sequence to be displayed can vary among cells of the set. For example, the host cells of the set collectively encode, e.g., between 10³ to 10¹¹ different amino acid sequences to be displayed, e.g., between 10⁵ to 10¹¹ or 10⁶ to 10¹⁰. In one embodiment, the host cells of the set collectively encode at least 10³, 10⁴, 10⁵, 10⁶, 10⁷, 10⁸, or 10⁹ different amino acid sequences to be displayed.

The amino acid sequence to be displayed may be unstructured, partially structured, or structured, e.g., it can include one or more structured domains. Typically the amino acid sequence to be displayed includes at least one folded domain, e.g., an immunoglobulin variable domain sequence or a Kunitz domain. One or more amino acid positions in the domain can vary among cells of the set.

In one embodiment, the second polypeptide is invariant for all host cells of the set. In one embodiment, the second polypeptide does not include a non-phage sequence of greater than five or twenty amino acids in length. For example, the second polypeptide can only include phage sequences.

In one embodiment, the first condition increases activity of the regulatable promoter relative to a reference condition (e.g., a standard condition provided herein), and the phage particles produced by the first set of host cells are characterized by a first average number of copies of the first polypeptide.

In one embodiment, the first condition decreases activity of the regulatable promoter relative to a reference condition (e.g., a standard condition provided herein), and the phage particles produced by the first set of host cells are characterized by a first average number of copies of the first polypeptide.

In one embodiment, the first conditions results in a level of production of the second polypeptide such that at least, on average, the ratio between the first polypeptide and the second polypeptide is between 1:1 and 1:1.5, 2, 5, or 10, 1:2 and 1:3, 5, or 10, 1:1 and (1.5, 5, 5, or 10):1, or 1:2 and (1, 3, 5, or 10):1. Ratios greater than these examples, favoring either the first or the second polypeptide, can also be used. In one embodiment, on average, at least one second polypeptide is assembled into a phage particle.

In one embodiment, the phage coat protein is the gene III protein and the phage particles produced have on average 1-2 copies of the second polypeptide and 3-4 copies of the first polypeptide.

In another embodiment, the phage coat protein is the gene III protein and the phage particles produced have on average 2-3 copies of the second polypeptide and about 2-3 copies of the first polypeptide.

In yet another embodiment, the phage coat protein is the gene III protein and the phage particles produced have on average 3-4 copies of the second polypeptide and 1-2 copies of the first polypeptide.

In another embodiment, the phage coat protein is the gene III protein and the phage particles produced have on average 4-5 copies of the second polypeptide and 0-1 copies of the first polypeptide. A titration of an inducing agent or other variable can be used to identify parameters of the condition which causes such particle assembly.

In one embodiment, the first expression unit is a component of a nucleic acid element that further includes a phage origin of replication and a phage packaging signal. For example, the nucleic acid element is a phagemid or a phage genome. In one embodiment, the first expression unit and the second expression unit are components of the same nucleic acid molecule, e.g., a phage genome.

In one embodiment, the first expression unit and the second expression unit are on separate nucleic acid molecules. For example, the first expression unit is on a nucleic acid molecule that can be packaged into a phage particle. The second nucleic acid unit can be on a different phage nucleic acid (e.g., the genome of a helper phage), on a plasmid in a host cell, or integrated into a chromosome in the host cell.

In one embodiment, the first polypeptide includes an immunoglobulin variable domain sequence (e.g., a heavy chain variable domain sequence). The first polypeptide can further include an immunoglobulin constant domain in frame with the immunoglobulin variable domain sequence. For example, the first polypeptide can include VH and CH1.

In one embodiment, the first expression unit further comprises an additional open reading frame, e.g., an open reading frame that is not in frame with the first open reading frame. Transcription of the first expression unit can, e.g., provide a transcript that includes both the additional open reading frame and the first open reading frame. In one embodiment, the first open reading frame encodes an immunoglobulin variable domain sequence (e.g., a heavy chain variable domain sequence), and the additional open reading frame also encodes an immunoglobulin variable domain sequence, particularly one compatible with the first (e.g., a light chain variable domain sequence). In other related embodiments, the first open reading frame and the additional open reading frame (or more) are used to encode respective subunits of a multi-chain protein. Accordingly the produced phage particles can display a Fab. Using a different configuration the particle can display a single chain antibody.

In one embodiment, the first polypeptide includes a mature full-length coat protein. For example, if the coat protein is gene III, the first polypeptide includes the mature full-length gene III protein. In an embodiment, the first polypeptide only includes a portion of the coat protein. For example, if the coat protein is gene III protein, the first polypeptide includes only the anchor domain of gene III protein.

In an embodiment in which the coat protein is required for infection, the second polypeptide includes at least sufficient sequences from the coat protein to enable formation of infectious particles. For example, if the coat protein is the gene III protein, the second polypeptide can include at least the N- and C-terminal domains of the gene III protein. In one embodiment, the second polypeptide includes a mature full-length coat protein.

In one embodiment, the filamentous phage is selected from the group consisting of M13, fl, and fd. For example, the portion of the coat protein in the first and second open reading frame is a portion of the gene III protein. In one embodiment, the gene III protein is a wild-type gene III protein (e.g., glycine at position 358). In another embodiment, the gene III protein is a mutant or variant of gene III protein that physically associates with phage particles less efficiently than wild-type.

In one embodiment, the first and second polypeptides include, at least, the same segment of a particular coat protein. For example, the first polypeptide can include the anchor domain of gene III protein, and the second polypeptide can include the mature, full-length gene III protein. In one embodiment, the common portion of the coat protein in the first or second open reading frame is encoded by at least one synthetic codon. For example, a segment of at least 20, 50, 70, or 150 amino acids in the portion of the coat protein is identical in the first and second polypeptide, but the nucleic acid sequence encoding the segment differs by at least one nucleotide (e.g., at least 5, 10, 20, 50, or 70) in the first open reading frame relative to the second open reading frame. Different nucleic acids can encode the same amino acid segment, but use of different codons. For example, the sequence encoding of the segment in the first open reading frame can use natural codons from the phage gene, whereas the sequence encoding of the segment in the second open reading frame can use synthetic codons. The configuration can be reversed, or each open reading frame can include synthetic codons, e.g., different synthetic codons, or synthetic codons at different positions.

In one embodiment, activity of the second promoter is regulated by an agent, and the first condition includes presence of the agent. Generally, the first and second promoter differ at least such that an agent or other intervention that regulates the second promoter does not cause a commensurate change to activity of the first promoter. For example, the second promoter regulatable by the lacI repressor, e.g., the second promoter is a lac promoter or a synthetic lacI-regulated promoter (e.g., tac).

In one embodiment, the first promoter is constitutive. For example, the first promoter is a phage promoter. In one embodiment, the phage promoter is a promoter naturally associated with an open reading frame encoding phage coat protein.

In one embodiment, the first promoter has a lower baseline activity than the second promoter, e.g., under standard conditions described herein. In one embodiment, the first promoter is less active than the lac promoter.

In one embodiment, the method further includes: selecting a subset of the phage particles produced by the set (e.g., a first set) of the host cells, introducing nucleic acid from phage particles of the subset into a second set of bacterial host cells, maintaining at least two host cells of the second set under a second condition. Use of the second condition results in a different level of activity of the second promoter than the first condition. Accordingly, phage particles produced by the second set of host cells are characterized by a second average number of copies of the first polypeptide physically attached to the phage, and the second average number of copies is different from the first average number of copies. For example, the second average number of copies is less than the first average number of copies.

The selecting can be based on a functional criteria, e.g., binding, enzymatic activity, stability, etc., and combinations thereof. In one embodiment, the selecting includes contacting phage to a target (e.g., a target molecule or target cell), and separating phage that bind the target from phage that do not bind the target. The target can be immobilized, e.g., prior, during or after the contacting.

In one embodiment, the method can further include selecting a subset of the phage particles produced by host cells of the second set.

In one embodiment, the method (e.g., using just a first set of host cells, or using both a first and second set) further includes administering a protein displayed by a selected phage or a functional segment thereof to a cell or an organism (e.g, a mammal, e.g., a rodent or human). In one embodiment, the method further includes formulating a protein displayed by a selected phage or a functional segment thereof for administration to an organism, e.g., as a pharmaceutically acceptable composition. In one embodiment, the method further includes varying the protein or functional segment thereof, and administering a variant to a cell or organism, or formulating the variant for administration, e.g., as a pharmaceutically acceptable compostion. In one embodiment, the method further includes sending or receiving information (e.g. nucleic acid or amino acid sequence information, or assay information (e.g., binding information) about a protein displayed by a selected phage or a functional segment thereof

In another aspect, the invention features a host cell that includes: a) a first expression unit including (1) a first open reading frame and (2) a first promoter operably linked to the first open reading frame, wherein the first open reading frame encodes a first polypeptide including (i) an amino acid sequence to be displayed on a phage and (ii) a portion of a phage coat protein, the portion of the phage coat protein being capable of physically associating with phage particles, and b) a second expression unit including (1′) a second open reading frame and (2′) a second promoter that is regulatable and operably linked to the second open reading frame. The second open reading frame encodes a second polypeptide including a portion of the phage coat protein. The portion of the phage coat protein is capable of physically associating with phage particles.

The host cell can be a bacterial cell, e.g., a non-pathogenic bacterial cell, e.g., a Gram positive or Gram negative bacterial cell, e.g., an E. coli cell.

In one embodiment, the amino acid sequence to be displayed includes at least one folded domain, e.g., an immunoglobulin variable domain sequence or a Kunitz domain. One or more amino acid positions in the domain can vary among cells of the set.

In one embodiment, the second polypeptide does not include a non-phage sequence of greater than five or twenty amino acids in length. For example, the second polypeptide can only include phage sequences.

In one embodiment, the first expression unit is a component of a nucleic acid element that further includes a phage origin of replication and a phage packaging signal. For example, the nucleic acid element is a phagemid or a phage genome. In one embodiment, the first expression unit and the second expression unit are components of the same nucleic acid molecule, e.g., a phage genome.

In one embodiment, the first expression unit and the second expression unit are on separate nucleic acid molecules. For example, the first expression unit is on a nucleic acid molecule that can be packaged into a phage particle. The second nucleic acid unit can be on a different phage nucleic acid (e.g., the genome of a helper phage), on a plasmid in a host cell, or integrated into a chromosome in the host cell.

In one embodiment, the first polypeptide includes an immunoglobulin variable domain sequence (e.g., a heavy chain variable domain sequence). The first polypeptide can further include an immunoglobulin constant domain in frame with the immunoglobulin variable domain sequence. For example, the first polypeptide can include VH and CH1.

In one embodiment, the first expression unit further comprises an additional open reading frame, e.g., an open reading frame that is not in frame with the first open reading frame. Transcription of the first expression unit can, e.g., provide a transcript that includes both the additional open reading frame and the first open reading frame. In one embodiment, the first open reading frame encodes an immunoglobulin variable domain sequence (e.g., a heavy chain variable domain sequence), and the additional open reading frame also encodes an immunoglobulin variable domain sequence, particularly one compatible with the first (e.g., a light chain variable domain sequence).

In one embodiment, the first polypeptide includes a mature full-length coat protein. For example, if the coat protein is gene III, the first polypeptide includes the mature full-length gene III protein. In an embodiment, the first polypeptide only includes a portion of the coat protein. For example, if the coat protein is gene III protein, the first polypeptide includes only the anchor domain of gene III protein.

In an embodiment in which the coat protein is required for infection, the second polypeptide includes at least sufficient sequences from the coat protein to enable formation of infectious particles. For example, if the coat protein is the gene III protein, the second polypeptide can include at least the N- and C-terminal domains of the gene III protein. In one embodiment, the second polypeptide includes a mature full-length coat protein.

In one embodiment, the filamentous phage is selected from the group consisting of M13, fl, and fd. Filamentous phage coat proteins such as the gene III, gene VI, gene VII, gene VIII, and gene IX proteins or portions of these proteins (e.g., functional portions) can be used. For example, the portion of the coat protein in the first and second open reading frame is a portion of the gene III protein. In one embodiment, the gene III protein is a wild-type gene III protein (e.g., glycine at position 358). In another embodiment, the gene III protein is a mutant or variant of gene III protein that physically associates with phage particles less efficiently than wild-type.

In one embodiment, the first and second polypeptides include, at least, the same segment of a particular coat protein. For example, the first polypeptide can include the anchor domain of gene III protein, and the second polypeptide can include the mature, full-length gene III protein.

In one embodiment, the codons encoding the coat protein domain of the first polypeptide or the second polypeptide are synthetic, i.e., the naturally occurring codons are altered so as to prevent recombination with sequences encoding the endogenous coat protein or with sequences encoding the coat protein domain of the second polypeptide. For example, the second polypeptide includes the full length mature gene III protein, e.g., encoded by at least two non-naturally occurring codons. In one embodiment, the second polypeptide is free of non-phage amino acid sequences, e.g., free of a mammalian amino acid sequence or a sequence from a source other than the bacteriophage in use. In another embodiment, the second polypeptide contains less than 30, 20, 10, 5, or 2 amino acids derived from a non-phage amino acid sequence, e.g., exogenous amino acid sequences.

In one embodiment, the common portion of the coat protein in the first or second open reading frame is encoded by at least one synthetic codon. For example, a segment of at least 20, 50, 70, or 150 amino acids in the portion of the coat protein is identical in the first and second polypeptide, but the nucleic acid sequence encoding the segment differs by at least one nucleotide (e.g., at least 5, 10, 20, 50, or 70) in the first open reading frame relative to the second open reading frame. Different nucleic acids can encode the same amino acid segment, but use of different codons. For example, the sequence encoding of the segment in the first open reading frame can use natural codons from the phage gene, whereas the sequence encoding of the segment in the second open reading frame can use synthetic codons. The configuration can be reversed, or each open reading frame can include synthetic codons, e.g., different synthetic codons, or synthetic codons at different positions.

In one embodiment, the first and second promoter differ at least such that an agent or other intervention that regulates the second promoter does not cause a commensurate change to activity of the first promoter. For example, the second promoter regulatable by the lacI repressor, e.g., the second promoter is a lac promoter or a synthetic lacI-regulated promoter (e.g., tac). The activity of a second promoter can be modulated (e.g., increased or decreased) relative to a reference level, e.g., induced or suppressed. For example, promoter activity can be altered by a factor of at least 1.1, 1.2, 1.5, 1.8, 2.0, 2.5, 5, 6, 10, 50, or 100 fold relative to the reference level (e.g., a standard condition described herein). In one embodiment, the second promoter is not endogenous to the phage. The second promoter can be regulated, for example, by an environmental parameter, e.g., a thermal change, pH change, nutrient change, hormones, metals, metabolites, antibiotics, or chemical agents. Exemplary inducible promoters include lac, tet, trp, tac, rho, ara, and rhamnose promoters. In one embodiment, the inducible promoter is a lac promoter. The lac promoter is positively regulated by lactose and molecules that are structurally related to lactose (e.g., allolactose), and is negatively regulated by glucose and molecules that are structurally related to glucose. In another embodiment, a promoter can be indirectly regulated.

In one embodiment, the first promoter is constitutive. For example, the first promoter is a phage promoter. In one embodiment, the phage promoter is a promoter naturally associated with an open reading frame encoding phage coat protein. In another embodiment, the first promoter is not regulatable (e.g., the activity of the first promoter is not significantly altered by an environmental parameter, such as the environmental parameter that alters activity of the regulatable parameter).

In one embodiment, the first promoter has a lower baseline activity than the second promoter, e.g., under standard conditions described herein. In one embodiment, the first promoter is less active than the lac promoter.

In another aspect, the invention features a nucleic acid that includes: a) a first expression unit including (1) an open reading frame and (2) a first promoter operably linked to the open reading frame, wherein the open reading frame encodes a first polypeptide including (i) an amino acid sequence to be displayed and (ii) a portion of a phage coat protein, the portion of the phage coat protein being capable of physically associating with phage particles, and b) a second expression unit including a (1′) second open reading frame and (2′) a second promoter that is regulatable and operably linked to the second open reading frame. The second open reading frame encodes a second polypeptide including a portion of the phage coat protein. The portion of the phage coat protein is capable of physically associating with phage particles. The nucleic acid can be a phage genome. The nucleic acid can include other features described herein.

In another aspect, the invention features plurality of phage particles produced by a method described herein.

In another aspect, the invention features a library of host cells. The library includes plurality of host cells, e.g., as described herein (e.g., above), wherein the amino acid sequence to be displayed varies among cells of the plurality. In one embodiment, the host cells of the plurality collectively encode, e.g., between 10³ to 10¹² different amino acid sequences to be displayed, e.g., between 10⁵ to 10¹¹ or 10⁶ to 10¹⁰. In one embodiment, the host cells of the plurality collectively encode at least 10³, 10⁴, 10⁵, 10⁶, 10⁷, 10⁸, or 10⁹ different amino acid sequences to be displayed.

In another aspect, the invention features a library of phage particles. The library includes a plurality of phage particles that include a phage genome, e.g., as described herein. The amino acid sequence to be displayed varies among phage particles of the plurality. In one embodiment, the phage particles of the plurality collectively encode between 10³ to 10¹² different amino acid sequences to be displayed, e.g., between 10⁵ to 10¹¹ or 10⁶ to 10¹⁰. In one embodiment, the phage particles of the plurality collectively encode at least 10³, 10⁴, 10⁵, 10⁶, 10⁷, 10⁸, or 10⁹ different amino acid sequences to be displayed.

In another aspect, the invention features a phagemid that includes: a) an open reading frame that encodes a polypeptide including an amino acid sequence to be displayed and a portion of a phage coat protein, wherein the amino acid sequence to be displayed is a heterologous sequence, b) a promoter, operably linked to the open reading frame, wherein the promoter is (i) a phage promoter or (ii) a promoter that has less than 70, 60, 50, 40, 30, 20, 10, or 5% of the activity of the lac promoter in Luria Broth at 30 or 37° C., c) a phage origin of replication, and d) a phage packaging signal.

In one embodiment, the promoter is a phage promoter that is naturally associated with an open reading frame encoding the phage coat protein.

In one embodiment, the amino acid sequence to be displayed includes an immunoglobulin variable domain sequence.

In another aspect, the invention features a kit that includes: (a) the phagemid described herein or a phage particle or cell that contains the phagemid; and (b) an isolated nucleic acid that includes a nucleic acid sequence that includes an open reading frame that encodes a polypeptide including a portion of a phage coat protein and a regulatable promoter, operably linked to the open reading frame, or a phage particle or cell containing the nucleic acid.

In another aspect, the invention features phagemid including: a display cassette configured to receive a sequence encoding an amino acid sequence to be displayed; a sequence encoding at least a portion of a phage coat protein; and a promoter that is identical, or substantially identical to an endogenous phage promoter, or includes a sequence that hybridizes to a strand of an endogenous phage promoter, the promoter being operably linked to the display cassette such that a transcript can be produced that includes a sequence inserted into the display cassette and the sequence encoding at least a portion of the phage coat protein. In one embodiment, the phagemid is less than 12, 11, 10, or 9 kilobases. The phagemid can include other features described herein.

In another aspect, the invention features a phagemid that includes: a coding sequence encoding a polypeptide that includes a first amino acid sequence to be displayed and at least a portion of a phage coat protein; and a promoter that is identical, or substantially identical to an endogenous phage promoter, or includes a sequence that hybridizes to a strand of an endogenous phage promoter, the promoter being operably linked to the coding sequence. In one embodiment, the phagemid further includes a second coding sequence that encodes a second amino acid sequence to be displayed, wherein the second amino acid sequence is not attached to a portion of phage coat protein, but can associate with the first amino acid sequence. In one embodiment, the first amino acid sequence includes a first immunoglobulin variable domain sequence, and the second amino acid sequence includes a second immunoglobulin variable domain sequence that can interact with the first immunoglobulin variable domain sequence to form an antigen binding site. The phagemid can include other features described herein.

In one embodiment, the invention features a method of providing phage particles that display a heterologous amino acid sequence, the method including: providing a host cell that includes the phagemid as described herein, and a genome of a helper phage, the genome including a regulatable promoter operably linked to a sequence encoding a coat protein whose abundance in the cell modulates incorporation of the amino acid sequence to be displayed into phage particles; and maintaining the host cell under conditions, whereby phage particles that package the phagemid are produced. In one embodiment, the conditions are selected to alter activity of the regulatable promoter relative to a reference activity level of the regulatable promoter.

In another aspect, the invention features an polypeptide (e.g., an isolated polypeptide) that includes a portion of a filamentous phage gene III protein, wherein the polypeptide can incorporate into phage particles, and the efficiency of its incorporation is less than the efficiency of incorporation of wild-type. In one embodiment, the portion is the gene III protein c-terminal domain, and the polypeptide is altered at position 358 of gene III relative to wild-type. For example, the polypeptide includes a substitution mutation, e.g., a substitution at position G358, e.g., G358S, or at position L196, e.g., L196P.

The invention also features a nucleic acid that includes a sequence that encodes the polypeptide.

In another aspect, the invention features a filamentous display phage that includes (a) a display protein physically associated with the phage particle, and (b) a polypeptide that includes portion of a phage coat protein, wherein the polypeptide can incorporate into phage particles, but with an efficiency less than the efficiency of incorporation of a corresponding wild-type portion, and the polypeptide does not include a non-phage domain. The polypeptide that includes portion of a phage coat protein can be e gene III protein c-terminal domain. In one embodiment, the polypeptide is altered at position 358 of gene III relative to wild-type. For example, the polypeptide includes a substitution mutation, e.g., a substitution at position G358, e.g., G358S, or at position L196, e.g., L196P.

In another aspect, the invention features a library that includes a plurality of host cells, wherein each cell of the plurality is according to any of the host cells described herein, and the amino acid sequence to be displayed of the first polypeptide differs among cells of the plurality. For example, the plurality can encode between 10³ to 10¹² different display proteins. In one embodiment, the plurality of nucleic acid elements encodes between 10⁶ to 10¹⁰ different antibody variable domains.

In one embodiment, the amino acid sequence of the second polypeptide is invariant among the members of the library.

In one embodiment, the amino acid sequence of the first polypeptide differs among members of the library and the amino acid sequence of a third polypeptide differs among members of the library.

In one embodiment, the amino acid sequence of the first polypeptide differs among members of the library and the amino acid sequence of the third polypeptide does not differ among members of the library. In another embodiment, the amino acid sequence of the first polypeptide does not differ among members of the library and the amino acid sequence of the third polypeptide differs among members of the library.

The library can further include one or more features described herein.

In another aspect, the invention features a library of bacteriophage particles produced from the any of the host cells described herein, wherein a majority (e.g., more than 50%, 60%, 70&, 80%, 90%, or 95%) of the phage particles include the first polypeptide encoded by a nucleic acid element packaged therein. In one embodiment, the library includes between 10³ to 10¹² types of phage particles (e.g. phage particles having different amino acid sequences of the first polypeptide).

In another aspect, the invention features a method of producing phage particles, the method including: providing a plurality of host cells that include phagemids according to the phagemids described herein, introducing a helper phage into at least two host cells of the plurality, wherein the helper phage includes an expression unit that encodes at least portion of the coat protein operably linked to a regulatable promoter; and maintaining at least two host cells under conditions (e.g., achieving a desired degree of regulation) wherein the host cells produce infectious phage particles that package the phagemids. In some embodiment, host cells that do not include the phagemids can be present.

In one aspect, the invention features a method of providing a phage display library, the method including:

a) providing a plurality of diverse nucleic acids, the plurality containing at least 10² different nucleic acid sequences that each encode a polypeptide of at least 6 amino acids,

b) generating a plurality of nucleic acid elements, each element containing a first expression unit including (1) a first open reading frame and (2) a first promoter operably linked to the first open reading frame. The first open reading frame that includes a coding sequence from the plurality of diverse nucleic acids and a sequence encoding a phage coat protein. Each nucleic acid element can further include a phage origin of replication and a phage packaging signal. For example, the nucleic acid element can be a phagemid.

The method can further include introducing nucleic acid elements from the plurality of nucleic acid elements into host cells to provide host cells that include the first expression unit. The host cells can include a second expression unit including (1′) a second open reading frame and (2′) a second promoter operably linked to the second open reading frame, wherein the second open reading frame encodes a second polypeptide including a portion of the phage coat protein, and wherein the second promoter is regulatable. The second expression unit can also be an invariant component of each of the nucleic acid elements. The method can further include: d) maintaining the host cells under conditions that produce phage particles that include at least the nucleic acid element and the first polypeptide attached the phage particles. In some embodiments, host cells may produce some particles that do not include the first polypeptide.

In one embodiment, the diverse nucleic acids include oligonucleotides, e.g., synthetic oligonucleotides.

In one embodiment, the generating includes joining nucleic acid fragments that contain the oligonucleotides into a vector element. The joining can include restriction digestion and ligation.

In one embodiment of the method, the diverse nucleic acids include cDNAs.

The method can further include one or more features described herein.

In another aspect, the invention features a method of preparing a population of display phage, the method including: (i) providing a first population of phage, wherein (a) each phage contains a nucleic acid that contains (1) a phage packaging signal, (2) a phage origin of replication, and (3) a first expression unit including (I) a first open reading frame that encodes a first polypeptide containing a display protein and a portion of a phage coat protein, (II) a first promoter operably linked to the first open reading frame, (b) the first population includes a plurality of phage that include the display protein physically attached, and (c) the abundance of the first polypeptide physically attached to the phage of the plurality is characterized by a first average number of copies (e.g., average valency); (ii) selecting, from the first population, a set of phage that bind to a target using the display protein; (iii) infecting cells with phage from the set of phage, the cells containing a second expression unit that includes (I′) a second open reading frame encodes a second polypeptide including a portion of the phage coat protein, portion being able to compete with the first polypeptide for incorporation into phage particles, and (II′) an regulatable promoter operably linked to second open reading frame; and (iv) producing a second population of phage from the cells under conditions that result in a plurality of phage that include the first polypeptide in an abundance characterized by a second average number of copies (e.g., average valency), different from the first average number of copies.

In one embodiment, the phage coat protein is the gene III protein of filamentous phage. In other embodiments, the phage coat protein is one of the phage coat proteins described herein.

In one embodiment, the display protein includes an immunoglobulin variable domain, e.g., a heavy chain variable domain, a light chain variable domain, a heavy chain variable domain and a light chain variable domain encoded in a single polypeptide

In one embodiment, the display protein includes an immunoglobulin variable domain and a gene III membrane anchor domain.

In one embodiment, the conditions repress the regulatable promoter.

In another embodiment, the conditions derepress or activate the regulatable promoter. Regulatable promoters include promoters that can be regulated, e.g., by metabolites or antibiotics.

In one embodiment, the regulatable promoter is the lac promoter.

In another embodiment, the regulatable promoter is regulated by a bacteriophage RNA polymerase whose expression is controlled by a second regulatable promoter, e.g., the regulatable promoter is regulated by a sigma factor whose activity is regulatable.

In one embodiment, the first promoter is a non-regulatable promoter, e.g., the first promoter is a natural promoter of the coat protein, or a constitutive promoter.

In one embodiment, the selecting includes forming phage-immobilized target complexes and separating phage that do not bind to the target from the phage-immobilized target complexes.

In one embodiment, the first average number of copies (e.g., valency) is greater than the second average number of copies, e.g., first average number of copies is at least two times greater than the second average number of copies, e.g., the first average number of copies is greater than four and the second average number of copies is less than two. In another related embodiment, the first average number of copies is greater than three and the second average number of copies is less than three.

In another embodiment, the second average number of copies is greater than the first average number of copies, e.g., the first average number of copies is less than three and the second average number of copies is greater than three. In another embodiment, the first average number of copies is less than two and the second average number of copies is greater than four.

In one embodiment, the second polypeptide is free of non-phage amino acid sequences. For example, the second polypeptide can be free of structured non-phage amino acid sequences (e.g., folded, non-phage domains).

In another aspect, the invention features a phage genome that includes an open reading frame and a promoter operably linked to the open reading frame, wherein the open reading frame encodes a polypeptide including a full length mature phage coat protein and no heterologous sequences, and the promoter is regulatable.

In another aspect, the invention features a phage genome having a display cassette operably linked to a DNA sequence that encodes at least a portion of a coat protein of the phage under control of the endogenous promoter corresponding to said coat protein and an auxiliary gene that has an regulatable promoter exogenous to the phage operably linked to an open reading frame which encodes a functional version of said coat protein.

In one embodiment, the genome also includes an exogenous selectable marker gene. In one embodiment, the phage is a filamentous phage, e.g., M13, fl, or fd. In one embodiment, the coat protein is picked from the group consisting of III, VIII, VI, VII, and IX. For example, the phage is M13, the coat protein is III, the regulatable promoter is PlacZ, and the phage contains an Ap^{R} gene.

In one embodiment, the display cassette includes two or more open reading frames such that one reading frame encodes a soluble protein and one reading frame encodes a display protein that associates with the soluble protein.

In another aspect, the invention features a phagemid having a display cassette operably linked to a DNA sequence that encodes at least a portion of a coat protein of the phage under control of the endogenous promoter corresponding to said coat protein. For example, the genome also includes an exogenous selectable marker gene.

In one embodiment, the phagemid is derived from a filamentous phage, e.g., M13, fl, and fd. In one embodiment, the coat protein is picked from the group consisting of III, VIII, VI, VII, and IX. For example, the parent phage is M13, the coat protein is III, and the phagemid contains an Ap^{R} gene. In one embodiment, the display cassette includes two or more open reading frames such that one reading frame encodes a soluble protein and one reading frame encodes a display protein that associates with the soluble protein. The invention also includes a library of phagemid wherein each genome is in accord with a phagemid described herein and the various phagemids differ in the DNA sequences that encoded the amino acid sequence to be displayed. In one embodiment, at least 1, 5, 10, 20, 25, 40, 50, or 70% of the phagemid particles display one or more copies of the polypeptide encoded by the display cassette. A similar library can be prepared using phage.

The invention also features nucleic acid vectors that include two or more elements (e.g., all elements) as shown in the Figures. In one embodiment, the vectors can be complete phage genomes, plasmids, or phagemids. In one embodiment, the elements are arranged in the same order as in the figures. In another embodiment, the order is altered. For example, one element can be place 5′ rather than 3′ of the other. Also, an element can be inverted, e.g., so transcription of the elements is in opposite direction (e.g., opposite convergent or divergent directions).

In another aspect, the invention features a method that includes: providing a set of host cells. Each of the host cells of the set includes a) a first expression unit and b) second expression unit. The first expression unit includes (1) a first open reading frame and (2) a first promoter operably linked to the first open reading frame. The first open reading frame encodes a first polypeptide including (i) an amino acid sequence to be displayed on a replicable genetic package (e.g., a phage or a cell) and (ii) an attachment sequence for attachment to the package. The second expression unit includes (1′) a second open reading frame, encoding a second polypeptide including an attachment sequence for attachment to the package or other factor which can modulate that attachment of the first polypeptide to the package, and (2′) a second promoter operably linked to the second open reading frame, wherein the second promoter is regulatable. The method can further include maintaining the set of host cells under a first condition, wherein packages (e.g., phage, other cells, or the host cells themselves) that include amino acid sequences to be displayed are produced. Methods for cell based display are described, e.g., in US 2003-0157091.

The term “phage” refers to a bacteriophage particle that includes a nucleic acid element such as a phagemid or a phage genome (e.g., a modified phage genome or a naturally occurring phage genome).

A “phage display package” or “phage display particle” refers to a phage particle that includes a heterologous protein accessible on the surface of the particle. The heterologous protein is typically attached by a covalent bond, e.g., a peptide bond or a non-peptide bond (e.g., a disulfide bond).

The term “heterologous,” when referring to a sequence, indicates that the sequence is not present in a particular context in nature. In the context of a phage, a sequence heterologous to the phage is does not naturally occur as an amino acid or nucleotide sequence of a respective naturally occurring filamentous phage. In the context of a cell, a sequence heterologous to the cell is does not naturally occur as an amino acid or nucleotide sequence of a respective naturally occurring cell. In the context of a fusion protein, a heterologous sequence does not occur in the same polypeptide sequence as a respective natural polypeptide. The sequence under consideration is typically is at least 10 amino acids or at least 20 nucleotides, e.g., the length of a relevant functional unit.

“Phagemid” means a replicable genetic construct that contains both a phage origin of replication and a phage-independent origin. Phagemids do not include a complete set of phage genes, e.g., sufficient number of genes to produce phage particles. Cells that harbor phagemid can produce phage-like particles that contain the phagemid genome when the cells are infected by a “helper” phage that carries requisite phage genes not present in the phagemid. A “display phagemid” is a phagemid that carries a gene encoding amino acids that can be displayed on the surface of a phage particle.

An “expression unit” is a nucleic acid sequence that includes a transcribable and translatable sequence that encodes a polypeptide. An expression unit can include a promoter, a ribosome binding site, a start codon, an open reading frame, and a stop codon. Optionally, an expression unit may contain an operator, i.e. a DNA sequence to which proteins or other molecules bind to alter the activity of the promoter. An expression unit can include a single open reading frame or a plurality of open reading frames. One exemplary type of expression unit functions in a eukaryotic cell, e.g., it includes requisite sequences adapted for the eukaryotic cell or the cell is adapted (e.g., by expression of a heterologous T7 polymerase gene).

The term “promoter” refers to a sequence at which transcription can be initiated by a RNA polymerase. Exemplary prokaryotic promoters include a polymerase binding site and optionally a site for sigma factor. Typical elements of one class of promoters is a −10 and −35 element. A promoter can be constitutive (i.e. always “on”) or regulatable (i.e. “on” only under certain conditions). In E. coli, promoters are between 30-50 basepairs in length, e.g., about 40 basepairs in length. One promoter is “highly homologous” to another promoter if they are identical (allowing insertion or deletion of up to 3 bases) at about 20 of 40 bases (e.g., at least 22, 24, 27, 30, 32, 34, 36, 37, 38, or 39), especially within the “−35 box” and the “−10 box”. Promoters are “similarly regulated” if they respond similarly. For example, similarly regulated promoters can respond in like manner to regulatory chemicals such as glucose, lactose, IPTG, cAMP, tryptophan, or other small molecules.

“Operably linked” means that the transcription of the open reading frame that is joined to the promoter is regulated at least to some measurable extent by the operably linked sequence, e.g., the transcriptional regulatory site, or the promoter.

The term “regulatable” promoter refers to a promoter whose activity can be modulated, e.g., by human intervention. For example, the activities of some promoters can be modulated by altering environmental conditions, e.g., adding or removing an inducer, changing temperature, pH, nutrients, etc. Promoters can be regulated by repressors and/or activators. Modulation of activity can be achieved, e.g., by increasing activator activity, decreasing activator activity, decreasing repressor activity (e.g., derepression), or increasing repressor activity. The term “inducing a promoter” refers to increasing promoter activity, regardless of mechanism (e.g., derepression or direct activation). Similarly, the term “suppressing promoter activity” refers to decreasing promoter activity, regardless of mechanism (e.g., direct repression or reduced activation).

A “display protein” is a protein that can be physically associate with phage particles, e.g., become integrated into a phage particle or otherwise be stably associated with the particle. The protein can include one or more polypeptide chains. It may only be necessary to directly associate one of the chains with the phage particle. For example, in the case of a Fab display protein, the polypeptide that includes a heavy chain immunoglobulin variable domain sequence can be associated with the particle, but not the polypeptide that includes the light chain immunoglobulin variable domain sequence, or vice versa. Embodiments described herein in the context of the display of a single chain display protein can be easily extended to the display of a multi-chain protein, e.g., as in the case of Fabs.

A “display cassette” is a nucleic acid sequence configured to receive an amino acid sequence to be displayed or is a nucleic acid that includes a sequence encoding an amino-acid sequence to be displayed, such as a peptide, a Kunitz domain, or an antibody Fab. An amino acid sequence to be displayed is typically a non-phage sequence, e.g., a sequence heterologous to a phage genome. A display cassette is said to be a “completed display cassette” if it includes the nucleic acid sequence encoding the amino acid sequence to be displayed. A nucleic acid sequence configured to receive an amino acid sequence to be display can include, e.g., a restriction enzyme polylinker or a site-specific recombinase site, or sequences for homologous recombination.

A “phage coat protein anchor segment” is that region of a phage coat protein that can be incorporated into or otherwise stably associated with a phage particle. For example, the anchor domain of the gene III protein of filamentous phage Fd is a phage coat protein anchor segment.

References to phage coat proteins, as described herein, encompass (i) wild-type phage coat proteins (including natural variants thereof), (ii) mutant phage coat proteins that have an amino acid sequence at least 80, 85, 87, 90, 92, 94, 95, 96, 97, 98, 99, or 99.5% identical to a corresponding wild-type coat protein and that are at least partially functional (e.g., able to assemble in a phage particle), and (iii) functional fragments of (i) and (ii). For example, the term “gene III protein” encompasses both the wild-type gene III protein and the S mutants (e.g., G358S in the c-terminal domain) described herein.

A “transformed cell” is a cell containing self replicating DNA that is foreign to the cell. Foreign DNA can be introduced by any method, e.g., electroporation, chemical transformation, or infection (e.g., phage infection).

Calculations of homology or sequence identity between sequences (the terms are used interchangeably herein) are performed as follows.

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. The percent identity between two amino acid or nucleotide sequences can be determined using the algorithm of Needleman and Wunsch ((1970) J. Mol. Biol. 48:444-453) algorithm which has been incorporated into the GAP program in the GCG software package, using either a Blossum 62 matrix and a gap weight of 12, a gap extend penalty of 4, and a frameshift gap penalty of 5.

Generally, to determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The invention encompasses nucleic acids that include features that are at least 50, 55, 60, 65, 70, 75, 80, 85, 90, 92, 93, 94, 95, 96, 97, 98, or 99% identical to features described herein and nucleic acid vectors that are at least so identical.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. The invention includes nucleic acids that hybridize with low, medium, high, or very high stringency to a nucleic acid described herein or to a complement thereof. The nucleic acids can be the same length or within 30, 20, or 10% of the length of the reference nucleic acid. The invention encompasses nucleic acids that include a stand that hybridizes to a nucleic acid that includes a feature described herein under low, medium, high, and very high stringency and nucleic acid vectors that include a stand that similarly hybridizes.

Some embodiments described herein provide, among other things, the advantage of more uniform control of valency. The regulatable promoter is typically arranged so that it does not directly control levels of the display protein, but rather the level of the wild-type coat protein that competes with the display protein for incorporation into phage particles. In a library, different display proteins can be expressed to varying degrees, for example, as a result of rare codons, secondary structures in RNAs, and so forth. However, in the indirect regulation design, the regulatable promoter drives expression of a protein that does not vary among members of the library. In other words, this valency control unit can be constant among members of the library, and, as such, be used to produce more uniform control of valency. Repression of the regulatable promoter allows creation of a high display-protein copy number (high valency) while activation of this regulatable promoter decreases the display protein by providing more of the wild-type coat protein.

In selecting binders to a target molecule in the first stage, a high copy number (valency) will be useful to retrieve as many amino acid sequences (binders) that show an interaction with the target molecule as possible. In a second step, one can select on basis of affinity (highest affinity binders). For this, a lower display level (valency) of the amino acid sequence to be displayed may be used. This is performed by activation of the regulatable promoter that drives the wild-type protein and competes with the display protein for incorporation into the phage (or phagemid) particles. The systems described here allow control over the display level on a phage coat by competition between phage coat protein (portion or full length version) controlled by a regulatable promoter and polypeptide comprising displayed sequence fused to the phage coat protein (portion or full length version) controlled by the endogenous promoter associated with that coat protein.

Other features and advantages of the instant invention will become more apparent from the following detailed description and claims. Embodiments of the invention can include any combination of features described herein. The contents of all references, pending patent applications and published patents, cited throughout this application are hereby expressly incorporated by reference, inclusive of Serial No. 60/429,134, filed on Nov. 26, 2002, US 2003-0157091, US 2003-0129659, US 20030157091 and U.S. Ser. No. 10/383,902.

### DESCRIPTION OF DRAWINGS

FIG. 1 is a schematic depiction of exemplary phage display DNA vectors, or portions of the phage display DNA vectors described herein, showing features that allow regulation of polypeptide expression. FIG. 1A depicts a portion of pRH04. FIG. 1B depicts a portion of pRH05. FIG. 1C depicts pRH06 and pRHO6-S. FIG. 1D depicts a portion of pDY3F31. FIG. 1E depicts a portion of DY3F63. FIG 1F depicts a portion of pDY3F39. FIG. 1G depicts a portion of pRH07. “PlacZ” refers to the LacZ promoter. “PgeneIII” refers to the natural promoter of the filamentous phage gene III protein. “Stump gene III” refers to the anchor domain of the gene III protein. “Fab cassette” refers to a nucleic acid segment encoding a polypeptide including an antibody variable domain.

FIG. 2 is a graph of the antibody display efficiency of phage expressing pRH04 and pDY3F31.

FIG. 3 is a graph of the display efficiency of phage expressing pRH05, pCES1, and pDY3F31 from a particular experiment.

FIG. 4 is a graph of the display and binding levels of phage expressing pRH05 compared with pRH06(s) from a particular experiment.

FIG. 5 is a graph of the display efficiency of phage expressing pRH06(s) and pRH05 from a particular experiment.

FIG. 6 is a schematic of pRH06.

FIG. 7 is a schematic of pRH07.

FIGS. 8A and 8B is an alignment of exemplary gene III protein sequences.

### DETAILED DESCRIPTION

Phage display libraries can be used to select proteins that bind a particular target molecule or cell. Phage display libraries are collections of particles that display a varied amino acid sequence (“display protein” or portion thereof) on the particle surface and contain the nucleic acid encoding the display protein packaged inside. The physical association between the display protein and the corresponding nucleic acid that encodes it enables the rapid isolation of target-binding protein molecules. Phage display libraries can be used, e.g., to identify useful antibodies, Kunitz domains, peptides, enzymes, and variants of virtually any protein.

The invention includes a method of controlling the copy number, e.g., valency, of display proteins on phage particles without obligatory recloning steps. The ability to control valency facilitates rounds of selection in which the valency differs between the rounds. The valency of the display proteins can be increased to facilitate recovery of all display proteins that bind to a target, or the valency can be reduced to select one or more display proteins with the highest affinity for the target.

A change in valency can be achieved without nucleic acid manipulation (e.g., cloning or PCR), although, in some cases, such manipulations might be desirable (e.g., to introduce new mutations). The change can be achieved by maintaining host cells under environment conditions that differ from a reference condition, e.g., standard growth conditions such as growth in LB, M9, or 2×YT at 30° C. or 37° C.

In an embodiment in which the display protein includes an immunoglobulin domain, high valency of antibody fragments favors efficient recovery of binding antibodies but may not optimize for selection of the antibody fragments having the highest affinity for the target. Because the number of phage particles containing a particular antibody will be low in a large library, it is important to implement a method that enables high recovery of the particles that display binding antibodies. Once these particles are recovered in the initial stages of a library screen, they can be amplified under conditions that produce multiple progeny particles with a lower valency. These progeny particles can be used for subsequent selections. A low valency of antibody fragments facilitates selection of high affinity binders. In some implementations, low valency is less than three protein molecules per particle, e.g., two or one display protein molecules per particle. Similar scenarios are applicable to other types of display proteins.

In one embodiment, regulation of valency is achieved by using two proteins that both can physically associate with the phage particle. One is the display protein, which will varies in phage display library; the other is an “invariant regulatable coat protein” or fragment thereof. The term “regulatable” in the context of an “invariant regulatable coat protein” refers only to the fact that the expression of this coat protein competition can be regulated, e.g., by a promoter whose activity is regulatable. Typically, the invariant regulatable coat protein and the display protein compete for inclusion into phage particles. For example, they can both include a common portion of a phage coat protein, e.g., the gene III protein. In another example, however, they do not directly compete, but levels of the invariant regulatable coat protein affect the extent of inclusion of the display protein.

Phage particles generally incorporate a fixed number of copies of a given phage coat protein (although some variation in number may be possible). At least in the case where the invariant regulatable coat protein and the display protein compete for inclusion, the ratio of expression of the display protein to the invariant regulatable coat protein in the host cell during particle assembly determines the relative numbers of each incorporated in the particles. Regulation of valency is achieved by regulating the ratio, in particular by controlling transcription of the nucleic acid encoding the invariant regulatable coat protein.

The invariant regulatable coat protein is typically a full-length mature phage coat protein. However, a protein that includes only a function portion, e.g., a domain that inserts into the phage coat, can also be used. For example, the gene III anchor domain can be used to compete with a display protein that also include a gene III anchor domain. In some implementations, the invariant regulatable coat protein can, if desired, include one or more heterologous amino acids that are inert and do not interfere with the display protein. In other implementations, the invariant regulatable coat protein does not include any heterologous sequences, e.g., no non-phage sequences.

A nucleic acid can be constructed that operably links a regulatable promoter and a sequence encoding the invariant regulatable coat protein. Use of a regulatable promoter that responds to changes in environmental conditions enables a user to selectively produce phage particles under conditions that favor (a) increased invariant regulatable coat protein expression and low valency or (b) decreased invariant regulatable coat protein expression and high valency.

Regulatable Promoters

Many regulatable (e.g., inducible or repressible) promoters are known. Such promoters include promoters whose activity can be altered or regulated by the intervention of a user, e.g., by manipulation of an environmental parameter. For example, an exogenous chemical compound can be added to regulate promoter activity. Regulatable promoters can contain a transcriptional regulatory sequence to which transcriptional activator or repressor proteins can bind and modulate transcription. Such sequences are also called transcription factor binding sites.

Synthetic promoters that include transcription factor binding sites (e.g., from natural proteins) can be constructed and used as regulatable promoters. It is also possible make a promoter regulatable by operably linking it to a regulatory sequence that operates at a distance from the promoter, e.g., a distance greater than 100 or 500 basepairs.

Examples of regulatable promoters include promoters responsive to an environmental parameter, e.g., thermal changes, hormones, metals, metabolites, antibiotics, or chemical agents. Regulatable promoters appropriate for use in E. coli include promoters which contain transcription factor binding sites from the lac, tac, trp, trc, and tet operator sequences, or operons, the alkaline phosphatase promoter (pho), an arabinose promoter such as an araBAD promoter, the rhamnose promoter, the promoters themselves, or functional fragments thereof (see, e.g., Elvin et al., 1990, Gene 37: 123-126; Tabor and Richardson, 1998, Proc. Natl. Acad. Sci. U.S.A. 1074-1078; Chang et al., 1986, Gene 44: 121-125; Lutz and Bujard, March 1997, Nucl. Acids. Res. 25: 1203-1210; D. V. Goeddel et al., Proc. Nat. Acad. Sci. U.S.A., 76:106-110, 1979; J. D. Windass et al. Nucl. Acids. Res., 10:6639-57, 1982; R. Crowl et al., Gene, 38:31-38, 1985; Brosius, 1984, Gene 27: 161-172; Amanna and Brosius, 1985, Gene 40: 183-190; Guzman et al., 1992, J. Bacteriol., 174: 7716-7728; Haldimann et al., 1998, J. Bacteriol., 180: 1277-1286). Inducible promoter systems such as lac promoters may be bound by repressor or inducer molecules. Lac promoters are induced by lactose or structurally related molecules such as isopropyl-beta-D-thiogalactoside (IPTG) and are repressed by glucose.

One type of regulatable promoter is an inducible promoter. An “inducible promoter” is a promoter whose activity can be increased relative to a baseline state, typically standard laboratory growth conditions, e.g., growth in LB, M9, or 2×YT at 30° C. or 37° C. The term “inducible promoters” is independent of mechanism. For example, some inducible promoters are induced by a process of derepression, e.g., inactivation of a repressor molecule, others are induced by direct activation. Exemplary inducible promoters can be induced so that expression is greater than 1.1, 1.2, 1.5, 2, 4, 5, 10, 12, 15, 20, 40, 50, 100, or 500 fold of the baseline expression.

Another type of regulatable promoter is a repressible promoter. An “repressible promoter” is a promoter whose activity can be decreased relative to a baseline state, typically standard laboratory growth conditions, e.g., growth in LB, M9, or 2×YT at 30° C. or 37° C. The term “repressible promoters” is independent of mechanism. For example, some repressible promoters are induced by a process of inhibiting an activator protein, others are repressed by direct repression. Exemplary repressible promoters can be repressed so that expression is less than 70, 60, 50, 30, 25, 20, 10, 5, 3, 2, 1, 0.1% of the baseline expression. Some promoters are both inducible and repressible.

A regulatable promoter sequence can also be indirectly regulated. Examples of promoters that can be engineered for indirect regulation include: the phage lambda P_{R}, −P_{L}, phage T7, SP6, and T5 promoters. For example, the regulatory sequence is repressed or activated by a factor whose expression is regulated, e.g., by an environmental parameter. One example of such a promoter is a T7 promoter. The expression of the T7 RNA polymerase can be regulated by an environmentally-responsive promoter such as the lac promoter. For example, the cell can include an artificial nucleic acid that includes a sequence encoding the T7 RNA polymerase and a regulatory sequence (e.g., the lac promoter) that is regulated by an environmental parameter (Studier, F. W., and Moffatt, B. A. J Mol Biol. 189(1):113-30, 1986).The activity of the T7 RNA polymerase can also be regulated by the presence of a natural inhibitor of RNA polymerase, such as T7 lysozyme (Studier, F. W. J Mol Biol. 219(1):37-44, 1991).

In another example, the lambda P_{L} can be engineered to be regulated by an environmental parameter. For example, the cell can include a nucleic acid sequence that encodes a temperature sensitive variant of the lambda repressor. Raising cells to the non-permissive temperature releases the PL promoter from repression.

The regulatory properties of a promoter or transcriptional regulatory sequence can be easily tested by operably linking the promoter or sequence to a sequence encoding a reporter protein (or any detectable protein), e.g., lacZ or green fluorescent protein. This construct is introduced into a bacterial cell and the abundance of the reporter protein is evaluated under a variety of environmental conditions. A useful promoter or sequence is one that is selectively activated or repressed in certain conditions. Northerns can also be used, e.g., without using a reporter construct.

The nucleic acid sequence that encodes the display protein can be operably linked to a non-inducible promoter or a filamentous phage promoter. For example, the sequence encoding the display protein can be linked to the natural promoter of the phage coat protein to which the display is fused, such as the gene III protein promoter. The sequence encoding the display protein may also be operably linked to a constitutive promoter. Constitutive promoters include promoters that are constitutively active in the host cell in which the phage replicates.

In one aspect, control over the display protein is achieved indirectly by controlling the expression of the invariant coat protein polypeptide using a regulatable promoter. Competition for display on the coat of a phage particle between the regulatable, invariant coat protein polypeptide and the display protein (which is linked to a second copy of a portion of the coat protein) determines the valency of display.

The use of a regulatable promoter to direct expression of the invariant coat protein can allow more stringent control on the levels of the invariant coat protein than can be achieved with regulating the display proteins directly. This more stringent control over the levels of invariant coat protein can, in turn, result in more stringent control of the display protein. Control over the valency of the display protein and the invariant coat protein among the library members is useful since, in many cases, it facilitates the selection of library members that have a high affinity and high level of specificity for the target.

Coat Proteins

Phage display systems typically utilize Ff filamentous phage, such as phage fl, fd, M13, or other bacteriophages, such as T7 and lambdoid phages (see, e.g., Santini (1998) J. Mol. Biol. 282:125-135; Rosenberg et al. (1996) Innovations 6:1-6; Houshmet al. (1999) Anal Biochem 268:363-370; U.S. Pat. No. 5,223,409). In implementations using filamentous phage, for example, the display protein is physically attached to a phage coat protein anchor domain, and the level of the competing coat protein which typically includes the same anchor domain, but usually not a heterologous amino acid sequence is controlled by inducible expression. The competing coat protein can be the full length endogenous phage protein, although any protein can be used that competes with the phage coat protein anchor domain of the display protein for expression on the surface of the phage particle.

Phage coat proteins that can be used for protein display include (i) minor coat proteins of filamentous phage, such as gene III protein, and (ii) major coat proteins of filamentous phage such as gene VIII protein. Fusions to other phage coat proteins such as gene VI protein, gene VII protein, or gene IX protein can also be used (see, e.g., WO 00/71694). Portions (e.g., domains or fragments) of these proteins may also be used. Useful portions include domains that are stably incorporated into the phage particle, e.g., so that the fusion protein remains in the particle throughout a selection procedure.

In one embodiment, the anchor domain or “stump” domain of gene III protein used (see, e.g., U.S. Pat. No. 5,658,727 for a description of an exemplary gene III protein anchor domain). As used herein, an “anchor domain” refers to a domain that is incorporated into a genetic package (e.g., a phage). A typical phage anchor domain is incorporated into the phage coat or capsid.

In one embodiment, the protein that is used to modulate valency of the display protein includes a mutation that alters its efficiency of association with phage particles. For example, the mutation can alter (e.g., reduce) its ability to be assembled into phage particles relative to a corresponding wild-type protein. The mutation can include an insertion, deletion or substitution.

For example, the protein that is used to modulate valency of the display protein can include a mutation the c-terminal domain of the gene III protein that differs from wild-type. An exemplary c-terminal domain is as follows:

| | |
|---|---|
| TVESCLAKSH TENSFTNVWK DDKTLDRYAN YEGCLWNATG VVVCTGDETQ | (SEQ ID NO:14) |
| CYGTWVPIGL AIPENEGGGS EGGGSEGGGS EGGGTKPPEY GDTPIPGYTY | |
| INPLDGTYPP GTEQNPANPN PSLEESQPLN TFMFQNNRFR NRQGALTVYT | |
| GTVTQGTDPV KTYYQYTPVS SKAMYDAYWN GKFRDCAFHS GFNEDPFVCE | |
| YQGQSSDLPQ PPVNAGGGSG GGSGGGSEGG GSEGGGSEGG GSEGGGSGGG | |
| SGSGDFDYEK MANANKGAMT ENADENALQS DAKGKLDSVA TDYGAAIDGF | |
| IGDVSGLANG NGATGDFAGS NSQMAQVGDG DNSPLMNNFR QYLPSLPQSV | |
| ECRPFVFSAG KPYEFSIDCD KINLFRGVFA FLLYVATFMY VFSTFANILR | |

The above protein is altered at position 358 (numbering according to the total gene III sequence listing). The wild-type glycine is replaced with serine. It is also possible to replace the glycine with other non-serine residues, e.g. alanine or a hydrophobic residue, e.g., an aliphatic, e.g., valine. Other mutations can also be made in the c-terminal domain, e.g., within 10 or 5 amino acids of position 358. The domains can be evaluated for efficiency of incorporation into phage particles as described below.

For reference the wild-type, c-terminal domain is as follows:

| | |
|---|---|
| TVESCLAKSH TENSFTNVWK DDKTLDRYAN YEGCLWNATG VVVCTGDETQ | (SEQ ID NO:15) |
| CYGTWVPIGL AIPENEGGGS EGGGSEGGGS EGGGTKPPEY GDTPIPGYTY | |
| INPLDGTYPP GTEQNPANPN PSLEESQPLN TFMFQNNRFR NRQGALTVYT | |
| GTVTQGTDPV KTYYQYTPVS SKAMYDAYWN GKFRDCAFHS GFNEDPFVCE | |
| YQGQSSDLPQ PPVNAGGGSG GGSGGGSEGG GSEGGGSEGG GSEGGGSGGG | |
| SGSGDFDYEK MANANKGAMT ENADENALQS DAKGKLDSVA TDYGAAIDGF | |
| IGDVSGLANG NGATGDFAGS NSQMAQVGDG DNSPLMNNFR QYLPSLPQSV | |
| ECRPFVFGAG KPYEFSIDCD KINLFRGVFA FLLYVATFMY VFSTFANILR | |

The protein can also include the transmembrane and intracellular domain of gene III protein.

The display protein can be physically associated with the anchor domain via covalent, non-covalent, and non-peptide bonds. See, e.g., U.S. Pat. No. 5,223,409, Crameri et al. (1993) Gene 137:69 and WO 01/05950. The filamentous phage display systems typically encode the heterologous amino acid sequence as a fusion to a phage coat protein or anchor domain. For example, the phage can include a gene that encodes a signal sequence, the heterologous amino acid sequence, and the anchor domain, e.g., a gene III protein anchor domain.

A display protein can be initially translated with a signal sequence. U.S. Pat. No. 5,658,727 describes some exemplary signal sequences. Similarly a protein that inserts into a phage particle and modulates the valency of a display protein can also be initially translated with a signal sequence. An exemplary signal sequence is the pelB signal sequence or the native gene III protein signal sequence.

In one embodiment, the nucleic acid encoding the heterologous amino acid sequence that is operably linked to an inducible promoter includes synthetic codons that encode the coat protein domain. Such synthetic codons can be selected to prevent recombination between the nucleic acid sequence encoding the competing protein and the nucleic acid sequence encoding the display protein, which may use natural codons. The scenario can also be reversed, e.g., the nucleic acid encoding the display protein can use synthetic codons. It may be sufficient to include between 5% and 60%, or 20% and 50% synthetic codons. Also the nucleic acid encoding both proteins may include synthetic codons, e.g., in different regions, or in the same region, e.g., provided that the codons are sufficiently different to reduce recombination between the sequences.

Antibody-based methods such as ELISA can be used to measure the copy number of display protein on phage particles. For example, when the display protein includes an antibody domain, anti-immunoglobulin antibodies can be used to determine absorbance of antibody domains in samples containing a known concentration of phage. The concentration of antibody domains in these samples can be determined by comparison to standards, and the copy numbers of antibody per phage can be calculated by dividing this concentration by the phage titers (see, e.g., Nakayama et al., (1996) Immunotechnol 2:197-207).

Display Proteins

A display protein includes at least an amino acid sequence heterologous to the filamentous phage. The amino acid sequence can be, for example, synthetic or naturally occurring, e.g., mammalian, e.g., human. Synthetic amino acid sequences include variants of naturally occurring sequences, e.g., variants that are at least 30, 50, 70, 80, 90, 92, 94, 96, 97, 98, or 99% identical. The display protein is also physically attached to the genetic package and accessible to a probe. In the context of a display library, a display protein is varied at one or more amino acid positions, e.g., between 2 and 50 position or 5 and 24 positions. The number of unique display proteins represented in a library can be large (e.g., between 10³ to 10¹² different display proteins, or e.g., at least 10⁵, 10⁶, 10⁸ or 10⁹). Generally, a display protein can be at least 6, 12, 20, 45, 70, or 110 amino acids in length. In some embodiments, the display protein is less than 300, 200, 120, 60, or 25 amino acids in length.

Examples of display proteins include peptides, modified scaffold proteins, and particularly immunoglobulin domains.

The display protein can include, e.g., a peptide, e.g., an artificial peptide of 30 amino acids or less. The synthetic peptide can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least one disulfide bond, and, for example, a loop of about 4 to 12 non-cysteine residues (e.g., a loop length of less than 15, 12, or 9 amino acids). In one embodiment, the peptides are varied at one or more positions, e.g., non-cysteine positions.

The display protein can conform to a particular protein scaffold. Such proteins include diverse amino acid positions but also have features that dictate particular characteristics of the scaffold, such as invariant amino acid residues required for the molecule to adopt a three-dimensional structure. Examples of protein scaffolds include protease inhibitors, MHC molecules, extracellular domains such as fibronectin type III repeats and EGF repeats, TPR repeats, zinc finger domains, enzymes (e.g., proteases), signaling domains (e.g., SH2, SH3, PTB), toxins (e.g., conotoxins), and protease inhibitors (e.g., Kunitz domains). Scaffold proteins can be varied, e.g., at one or more positions, e.g., surface positions, functional positions (e.g., near or in an active site), or core positions.

In one embodiment, the display proteins are derived from heterodimeric receptors. Examples of such receptors include immunoglobulins (antibodies), major histocompatibility class I or II molecules, integrins, and T-cell receptors.

Immunoglobulin domains that can be used include immunoglobulin heavy chain variable domains (V_{H}), light chain variable domains (V_{L}), and heavy and light chains variable domains encoded in a single polypeptide chain. Variable immunoglobulin heavy and light chains can further include constant regions, e.g., CH1 or C_{L} domains. Methods of using immunoglobulin domains for display are known (see, e.g., Haard et al. (1999) J. Biol. Chem 274:18218-30; Hoogenboom et al. (1998) Immunotechnology 4:1-20. and Hoogenboom et al. (2000) Immunol Today 21:371-8). V_{H} and V_{L} domains can be expressed in lengths equal to, greater than, or less than their natural lengths. V_{H} and V_{L} domains will generally have less than 125 amino acid residues and usually more than 60 residues. The amino acid sequences of the V_{H} and V_{L} domains will vary greatly except for conserved cysteine residues separated by 60-75 amino acids which form a disulfide bond. Preparation of antibody variable domain libraries is known in the art (see, e.g., Huse et al. (1989) Science 246:1275-1281; Clackson et al. (1991) Nature 352:624-628; Hoogenboom et al. (1991) Nuc Acid Res 19:4133-4137). See below for further details on the construction of an exemplary antibody display library.

Nucleic Acid Constructs

Nucleic acid constructs can be engineered using standard methods of molecular biology. These methods can include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989).

In one aspect, the DNA sequences encoding both the invariant, regulatable coat protein and the display protein are on the same nucleic acid molecule. For example, both coding sequences can be contained in a circular nucleic acid, such as a phagemid or a modified phage genome. Alternatively, these DNA sequences can be on different nucleic acid molecules. For example, the sequence encoding the display protein can be contained in a phagemid, whereas the sequence encoding the regulatable coat protein can be integrated into the chromosome of the host cell or located on a plasmid separate from the phagemid.

Vectors may be constructed by standard cloning techniques to include a gene encoding a synthetic coat protein portion operably linked to an inducible promoter, and a gene encoding a heterologous amino acid sequence and the coat protein portion. One exemplary strategy to produce this type of vector includes modifying a phage genome to insert an inducible promoter in a position operably linked to an endogenous copy of the gene encoding the coat protein of interest.

An appropriate DNA vector can include restriction enzyme sites into which foreign sequences can be ligated, a nucleic acid sequence that can direct autonomous replication and maintenance in the appropriate host, and a gene whose expression provides a selective advantage to the host, such as an antibiotic resistance gene.

Phase Production and Screening

In one embodiment, the method includes amplifying a phage library member recovered in a selection for binders of a target compound. The method can be used to identify members of the phage library that interact with the target compound. In another embodiment, the method uses successive cycles such that phage displaying varied protein domains at a first valency are tested for interaction with a target compound, selected, amplified, and used to produce phage displaying varied protein domains at a second valency. This population is contacted to a target compound to select a subset of protein domains that bind under these conditions.

One exemplary method of screening and amplifying phage includes the following:

a. Contacting a plurality of diverse display phage to a target compound, wherein each phage of the plurality displays a varied heterologous amino acid sequence at a first valency;

b. Separating phage that bind to the target compound from unbound phage;

c. Infecting host cells with the bound phage;

d. Producing replicate phage from the infected cells in the presence of the target compound (“phage production”) under conditions that result in phage that display a heterologous amino acid sequence at a second valency;

e. Separating replicate phage that bind the target compound from the unbound phage;

f Repeating c. to e. one or more times, e.g., one to six times;

g. Recovering the bound phage, e.g., for individual characterization.

The host cells are maintained under conditions that provide a selected level of transcriptional activity of the inducible promoter during phage production. In an example in which the inducible promoter is a lac promoter, a lac inducer (e.g., IPTG), or an agent that inhibits activity of a lac promoter (e.g., glucose) can be included in the growth medium. In one embodiment, high concentrations of glucose (e.g., >1% ) are used. In another embodiment, low concentrations of glucose are used (e.g., <0.1% ). If temperature is not the factor used for induction, conditions for phage production may include a change in temperature. Lowering the incubation temperature for a specified time interval during phage production can facilitate folding of the display amino acid sequence, e.g., where the display amino acid sequence includes an immunoglobulin variable domain. One exemplary procedure for culturing host cells during phage production includes a 20 minute incubation period at 37° C. followed by a 25 minute incubation period at 30° C.

After any given cycle of selection, individual phage can be analyzed by isolating colonies on cells infected under low multiplicity of infection conditions. Each bacterial colony is cultured under conditions that result in production of low-valency phage, e.g., in microtiter wells. Phage are harvested from each culture and used in an ELISA assay. The target compound is bound to a well of microtiter plate and contacted with phage. The plates are washed and the amount of bound phage are detected, e.g., using an antibody to the phage.

In one aspect, the method pertains to the selection of phage that bind a target molecule. Any compound can serve as a target molecule. The target molecule may be a small molecule, a polypeptide, a nucleic acid, a polysaccharide, and so forth. Polypeptide target molecules can include small peptides (e.g., about 3 to 30 amino acids in length), single polypeptide chains, and multimeric polypeptides. These target molecules can be modified (e.g. glycosylated, ubiquitinated, phosphorylated, cleaved, disulfide bonded, and so forth). Polypeptide target molecules may have a specific physical conformation, e.g. a folded or unfolded form. Exemplary polypeptide targets include disease-associated polypeptides, cell surface proteins, hormones, cytokines, chemokines, cell surface receptors, virus receptors, and extracellular matrix binding proteins. It is also possible to use cells as a target. Cells present a complex array of molecules on their cell surface. Phage particles that bind specifically to the cells (e.g., relative to other cells) can be isolated.

Selection of phage that bind a target molecule includes contacting the phage to the target molecules. The target molecules can be bound to a solid support, either directly or indirectly. Phage particles that bind to the target are then immobilized and separated from members that do not bind the target. Conditions of the separating step can vary in stringency. Multiple cycles of binding and separation can be performed. Multiple cycles of binding and separation can be performed with phage that display a display amino acid sequence at a first valency (in some cycles) and a second valency (in other cycles).

The method can further include using the selected set of phage to infect host cells and produce a second population of phage. In one embodiment, the second population of phage is produced under conditions that result in a second valency of the display amino acid sequence. In the example when the inducible promoter is the lac promoter, the conditions can include inclusion of glucose or inclusion of IPTG in the growth medium.

In one embodiment, production of phage under conditions that repress the inducible promoter can maximize the valency of display (e.g., ligand-binding) polypeptides on the phage particle. In another embodiment, production of phage under conditions that derepress the inducible promoter can minimize the valency of ligand-binding polypeptides.

Covalent and non-covalent methods can be used to attach target molecules to a solid or insoluble support. Such supports can include a matrix, bead, resin, planar surface, or immunotube. In one example of a non-covalent method of attachment, target molecules are attached to one member of a binding pair. The other member of the binding pair is attached to a support. Streptavidin and biotin are one example of a binding pair that interact with high affinity. Other non-covalent binding pairs include glutathione-S-transferase and glutathione (see, e.g., U.S. Pat. No. 5,654,176), hexa-histidine and Ni²⁺ (see, e.g., German Patent No. DE 19507 166), and an antibody and a peptide epitope (see, e.g., Kolodziej and Young (1991) Methods Enz. 194:508-519 for general methods of providing an epitope tag).

Covalent methods of attachment of target compounds include chemical crosslinking methods. Reactive reagents can create covalent bonds between functional groups on the target molecule and the support. Examples of functional groups that can be chemically reacted are amino-, thiol-, and carboxyl- groups. N-ethylmaleimide, iodoacetamide, and N-hydrosuccinimide, and glutaraldehyde are examples of reagents that react with functional groups.

Display library phage can be selected or captured with a variety of methods. Phage can be captured by adherence to a vessel, such as a microtiter plate, that is coated with the target molecule. Alternatively, phage can contact target molecules that are immobilized within a flow chamber, such as a chromatography column. Phage particles can also be captured by magnetically responsive particles such as paramagnetic beads. The beads can be coated with a reagent that can bind the target compound (e.g., an antibody), or a reagent that can indirectly bind a target compound (e.g., streptavidin-coated beads binding to biotinylated target compounds).

The selection of library phage particles can be automated. Devices suitable for automation include multi-well plate conveyance systems, magnetic bead particle processors, liquid handling units, colony picking units, and other robotics. These devices can be built on custom specifications or purchased from commercial sources, such as Autogen (Framingham Mass.), Beckman Coulter (USA), Biorobotics (Woburn Mass.), Genetix (New Milton, Hampshire UK), Hamilton (Reno Nev.), Hudson (Springfield N.J.), Labsystems (Helsinki, Finland), Packard Bioscience (Meriden Conn.), and Tecan (Mannedorf, Switzerland).

In some cases, the methods described herein include an automated process for handling magnetic particles. The target compound is immobilized on the magnetic particles. The KINGFISHER™ system, a magnetic particle processor from Thermo LabSystems (Helsinki, Finland), for example, can be used to select display library members against the target. The display library is contacted to the magnetic particles in a tube. The beads and library are mixed. Then a magnetic pin, covered by a disposable sheath, retrieves the magnetic particles and transfers them to another tube that includes a wash solution. The particles are mixed with the wash solution. In this manner, the magnetic particle processor can be used to serially transfer the magnetic particles to multiple tubes to wash non-specifically or weakly bound library members from the particles. After washing, the particles can be transferred to a vessel that includes a medium that supports display library member amplification. In the case of phage display the vessel may also include host cells.

In some cases, e.g., for phage display, the processor can also separate infected host cells from the previously-used particles. The processor can also add a new supply of magnetic particles for an additional round of selection.

The use of automation to perform the selection can increase the reproducibility of the selection process as well as the through-put.

An exemplary magnetically responsive particle is the DYNABEAD® available from Dynal Biotech (Oslo, Norway). DYNABEADS® provide a spherical surface of uniform size, e.g., 2 μm, 4.5 μm, and 5.0 μm diameter. The beads include gamma Fe₂O₃ and Fe₃O₄ as magnetic material. The particles are superparamagnetic as they have magnetic properties in a magnetic field, but lack residual magnetism outside the field. The particles are available with a variety of surfaces, e.g., hydrophilic with a carboxylated surface and hydrophobic with a tosyl-activated surface. Particles can also be blocked with a blocking agent, such as BSA or casein to reduce non-specific binding and coupling of compounds other than the target to the particle.

The target is attached to the paramagnetic particle directly or indirectly. A variety of target molecules can be purchased in a form linked to paramagnetic particles. In one example, a target is chemically coupled to a particle that includes a reactive group, e.g., a crosslinker (e.g., N-hydroxy-succinimidyl ester) or a thiol.

In another example, the target is linked to the particle using a member of a specific binding pair. For example, the target can be coupled to biotin. The target is then bound to paramagnetic particles that are coated with streptavidin (e.g., M-270 and M-280 Streptavidin DYNAPARTICLES® available from Dynal Biotech, Oslo, Norway). In one embodiment, the target is contacted to the sample prior to attachment of the target to the paramagnetic particles.

In some implementations, automation is also used to analyze display library members identified in the selection process. From the final sample, individual clones of each display member can be obtained. Each member can be individually analyzed, e.g., to assess a functional property. Exemplary functional properties include: a kinetic parameter (e.g., for binding to the target compound), an equilibrium parameter (e.g., avidity, affinity, and so forth, e.g., for binding to the target compound), a structural or biochemical property (e.g., thermal stability, oligomerization state, solubility and so forth), and a physiological property (e.g., renal clearance, toxicity, target tissue specificity, and so forth) and so forth. Methods for analyzing binding parameters include ELISA, homogenous binding assays, and surface plasmon resonance. For example, ELISAs on a displayed protein can be performed directly, e.g., in the context of the phage or other display vehicle, or the displayed protein removed from the context of the phage or other display vehicle.

Each member can also be sequenced, e.g., to determine the nucleic acid sequence of the encoded protein that is displayed.

Methods of automation, including those described herein, can be used to analyze phage particles in which heterologous amino acid sequences expressed by the phage are characterized by a first valency in one set of cycles, and a second valency in another set of cycles.

See, e.g., US 2003-0129659 for additional automation methods.

Proteins identified from a display library or functional portions thereof can also be evaluated in a functional assay, e.g., for a biological function other than binding. For example, such proteins can be evaluated in a cell-based or organism-based assay. See, e.g., US 2003-0129659, US 20030157091 and U.S. Ser. No. 10/383,902 for exemplary functional assays.

Antibody Display Libraries

In one embodiment, the display library presents a diverse pool of polypeptides, each of which includes an immunoglobulin domain, e.g., an immunoglobulin variable domain. Display libraries are particular useful, for example for identifying human or “humanized” antibodies that recognize human antigens. Such antibodies can be used as therapeutics to treat human disorders such as cancer. Since the constant and framework regions of the antibody are human, these therapeutic antibodies may avoid being recognized and targeted as antigens. The constant regions are also optimized to recruit effector functions of the human immune system. The in vitro display selection process surmounts the inability of a normal human immune system to generate antibodies against self-antigens.

A typical antibody display library displays a polypeptide that includes a heavy chain immunoglobulin variable domain sequence and a light chain immunoglobulin variable domain sequence.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405). As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, or may include other alterations.

The display library can display the antibody as a Fab fragment (e.g., using two polypeptide chains) or a single chain Fv (e.g., using a single polypeptide chain). Other formats can also be used.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes (see, e.g., US 2002-0102613 and WO 00/70023). Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.

In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describes a method for constructing CDR encoding oligonucleotides using trinucleotide synthesis and a template with engineered restriction sites for accepting the oligonucleotides.

In another process, an animal, e.g., a rodent, is immunized with the MHC-peptide complex that includes a specific peptide or with a cell that presents a specific peptide on its surface bound to the MHC. The cell can have a particular allele of the MHC protein. The animal is optionally boosted with the antigen to further stimulate the response. Then spleen cells are isolated from the animal, and nucleic acid encoding VH and/or VL domains is amplified and cloned for expression in the display library. Of course, a display library may not need to be screened to obtain nucleic acids that encode antibodies specific for the target in this case.

In yet another process, antibody libraries are constructed from nucleic acid amplified from naive germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naïve.

Targets

Generally, any molecular species can be used as a target when evaluating a phage library described herein, e.g., a library of phage particles with a desired valency. The target can be of a small molecule (e.g., a small organic or inorganic molecule), a protein or polypeptide, a nucleic acid, cells, and so forth. By way of example, a number of examples and configurations are described for targets. Of course, targets other than, or having properties other, than those listed below can also be used.

One class of targets includes proteins. Examples of such targets include small peptides (e.g., about 3 to 30 amino acids in length), single polypeptide chains, and multimeric polypeptides (e.g., protein complexes).

A protein target can be modified, e.g., glycosylated, phosphorylated, ubiquitinated, methylated, cleaved, disulfide bonded and so forth. Preferably, the protein has a specific conformation, e.g., a native state or a non-native state. In one embodiment, the protein has more than one specific conformation. For example, prions can adopt more than one conformation. Either the native or the diseased conformation can be a desirable target, e.g., to isolate agents that stabilize the native conformation or that identify or target the diseased conformation.

In some cases, however, the protein is unstructured, e.g., adopts a random coil conformation or lacks a single stable conformation. Agents that bind to an unstructured protein can be used to identify the polypeptide when it is denatured, e.g., in a denaturing SDS-PAGE gel, or to separate unstructured isoforms of the protein for correctly folded isoforms, e.g., in a preparative purification process.

Some exemplary protein targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, or a cell surface protein (e.g., of a mammalian cancer cell or a pathogen). In some embodiments, the polypeptide is associated with a disease, e.g., cancer.

More specific examples include: integrins, cell attachment molecules or “CAMs” such as cadherins, selections, N-CAM, E-CAM, U-CAM, I-CAM and so forth); proteases, e.g., subtilisin, trypsin, chymotrypsin; a plasminogen activator, such as urokinase or human tissue-type plasminogen activator (t-PA); bombesin; factor IX, thrombin; CD-4; CD-19; CD20; platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin; mucins; human serum albumin; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; mullerian-inhibiting substance; gonadotropin-associated peptide; tissue factor protein; inhibin; activin; vascular endothelial growth factor; receptors for hormones or growth factors; protein A or D; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; immunoglobulin (constant or variable domains); and fragments of any of the above-listed polypeptides. In some embodiments, the target is associated with a disease, e.g., cancer.

The target protein is preferably soluble. For example, soluble domains or fragments of a protein can be used. This option is particularly useful for identifying molecules that bind to transmembrane proteins such as cell surface receptors and retroviral surface proteins.

Another class of targets includes cells, e.g., fixed or living cells. The cell can be bound to an antibody that is covalently attached to a paramagnetic particle or indirectly attached (e.g., via another antibody). For example, a biotinylated rabbit anti-mouse Ig antibody is bound to streptavidin paramagnetic beads and a mouse antibody specific for a cell surface protein of interest is bound to the rabbit antibody.

In one embodiment, the cell is a recombinant cell, e.g., a cell transformed with a heterologous nucleic acid that expresses a heterologous gene or that disrupts or alters expression of an endogenous gene. The heterologous nucleic acid can be under control of an inducible or constitutive promoter. In a preferred embodiment, the heterologous nucleic acid encodes a cell surface protein, e.g., a cell-surface protein of interest. The plasmid can also express a marker protein, e.g., for use in binding the transformed cell to a magnetically responsive particle.

In another embodiment, the cell is a primary culture cell isolated from a subject, e.g., a patient, e.g., a cancer patient. In still another embodiment, the cell is a transformed cell, e.g., a mammalian cell with a cell proliferative disorder, e.g., a neoplastic disorder. In still another embodiment, the cell is the cell of a pathogen, e.g., a microorganism such as a pathogenic bacterium, pathogenic fungus, or a pathogenic protist (e.g., a Plasmodium cell) or a cell derived from a multicellular pathogen. The target can also be a cell, e.g., a cancer cell, a hematopoietic cell, , and so forth.

In still another embodiment, the cells are treated (e.g., using a drug or genetic alteration). For example, the treatment can alter the rate of endocytosis, pinocytosis, exocytosis, and/or cell secretion. The treatment can also be a drug or an inducer of a heterologous promoter-subject gene construct. The treatment can cause a change in cell behavior, morphology, and so forth. Molecules that dissociate from the cells upon treatment or that associate with cells when treated are collected and analyzed.

In another embodiment, the target is a tissue or organ. The display library can be screened for members that bind to the tissue or organ in vitro or in vivo (e.g., as described in Kolonin et al. (2001) Current Opinion in Chemical Biology 5:308-313).

Additional exemplary targets include nucleic acids, e.g., double-stranded, single-stranded, and partially double-stranded DNA such as a site in a regulatory region, a site in a coding region, a tertiary structure e.g., a G-quartet or a telomere; RNA, e.g., double-stranded RNA, single-stranded RNA, e.g., an RNAi, a ribozyme; or combinations thereof. For example, a double stranded nucleic acid that includes a site can be used to identify a DNA-binding domain that binds to that site. The DNA-binding domain can be used in cells to regulate genes that are operably linked to the site. For example, the methods described herein can be used to screen a library of zinc finger polypeptides for binding to a target nucleic acid. See, e.g., Rebar et al. (1996) Methods Enzymol. 267:129-49 for a description of phage display libraries of zinc finger polypeptides.

Still more exemplary targets include organic molecules. In one embodiment, the organic molecules are transition state analogues and can be used to select for catalysts that stabilize a transition state structure similar to the structure of the analogue. In another embodiment, the organic molecules are suicide substrates that covalently attach to catalysts as a result of the catalyzed reaction.

A target can be a drug, e.g., a drug for which a ligand is required in order to improve purification of the drug, e.g., from a chemical reaction, a bioreactor, a media, milk, or a cell extract. The drug can include a peptide, e.g., a polypeptide or a non-peptide functionality.

Other targets may be relevant to biotechnological applications, e.g., to generate molecules useful for the laboratory. For example, streptavidin, green fluorescent protein, or a nucleic acid polymerase can be a target.

In some embodiments, more than one species is used as a target, e.g., a sample is exposed to a plurality of targets.

Therapeutic Uses

The methods described herein can be used to identify a protein with therapeutic properties. The protein can be used, e.g., for treatment, prophylaxis, general improvement with respect to a condition. The protein can be formulated with a pharmaceutically acceptable carrier to provide a pharmaceutical composition.

In another aspect, the present invention provides compositions, which include a target-specific binding protein, e.g., an antibody molecule, other polypeptide or peptide identified as binding to a target molecule using the method described herein, formulated together with a pharmaceutically acceptable carrier. Pharmaceutical compositions can encompass labeled binding proteins for in vivo imaging as well as therapeutic compositions.

As used herein, “pharmaceutically acceptable carriers” include any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein binding protein may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the target-specific binding protein is administered by intravenous infusion or injection. For example, for therapeutic applications, the target-specific binding protein can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the protein may be administered, for example, with an inert diluent or an assimilable edible carrier. The protein can be administered with medical devices known in the art. The protein can be administered, e.g., orally or parentally, to a subject, e.g., a mammal, e.g., a human.

Diagnostic Uses

Proteins identified by the screening methods described herein can be used to detect the target compound to which they bind, e.g., for detecting the presence of the target, in vitro (e.g., a biological sample, such as tissue, biopsy, e.g., a cancerous tissue) or in vivo (e.g., in vivo imaging in a subject). The following are merely exemplary uses of a target-specific binding protein. These include: ELISA assays, FACS analysis and sorting, microscopy, protein arrays, and in vivo imaging. These applications can be performed for one target-specific binding protein, or in a high-throughput mode for many such target-specific binding proteins.

A target specific binding protein can be labeled, e.g., using fluorophore and chromophore labeled protein binding proteins. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science, 162:526 and Brand, L. et al. (1972) Annual Review of Biochemistry, 41:843-868. The protein binding proteins can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein binding protein can be used to detect the presence or localization of the target molecule in a sample, e.g., using fluorescent microscopy (such as confocal or deconvolution microscopy).

Histological Analysis. Immunohistochemistry can be performed using the target-specific binding proteins identified by the methods described herein. The binding protein is labeled, and contacted to a histological preparation, e.g., a fixed section of tissue that is on a microscope slide. After an incubation for binding, the preparation is washed to remove unbound antibody. The preparation is then analyzed, e.g., using microscopy, to identify if the binding protein bound to the preparation.

Protein Arrays. A target-specific binding protein identified by a method described herein can be immobilized on a protein array. The protein array can be used as a diagnostic tool, e.g., to screen medical samples (such as isolated cells, blood, sera, biopsies, and the like). Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

In vivo Imaging. In still another embodiment, the target-specific binding proteins identified by the methods herein are conjugated to a detectable marker, administered to a subject, and imaged by detecting the detectable marker bound to target-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein binding protein can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Meth. Enzymol. 121: 802-816. NMR signals can be enhanced by contrast agents. Examples of such contrast agents include a number of magnetic agents paramagnetic agents (which primarily alter T1) and ferromagnetic or superparamagnetic (which primarily alter T2 response). The target-specific binding proteins can also be labeled with an indicating group containing of the NMR-active ¹⁹F atom. After permitting time for target binding, a whole body MRI is carried out using an apparatus such as one of those described by Pykett (1982) Scientific American, 246:78-88 to locate and image cancerous tissues.

Purification Uses

Proteins identified by the screening methods described herein can be used to purify a target compound. In one embodiment, the purification is on a production scale, e.g., to purify a protein pharmaceutical or other pharmaceutical. A target-specific binding protein identified by the methods herein can be couple to a support and used as an affinity reagent in affinity chromatography. Scopes (1994) Protein Purification: Principles and Practice, New York:Springer-Verlag provides a number of methods for purifying recombinant and non-recombinant proteins by affinity chromatography. The use of a customized target specific binding protein, particular one with high specificity, can obviate the need for an affinity tag, and/or can enable highly specific separation of closely related isoforms.

The following invention is further illustrated by the following non-limiting examples.

### EXAMPLE 1

### Construction of pRH04 Phase Display DNA Vector for Regulating Valency of Displayed Polypeptides

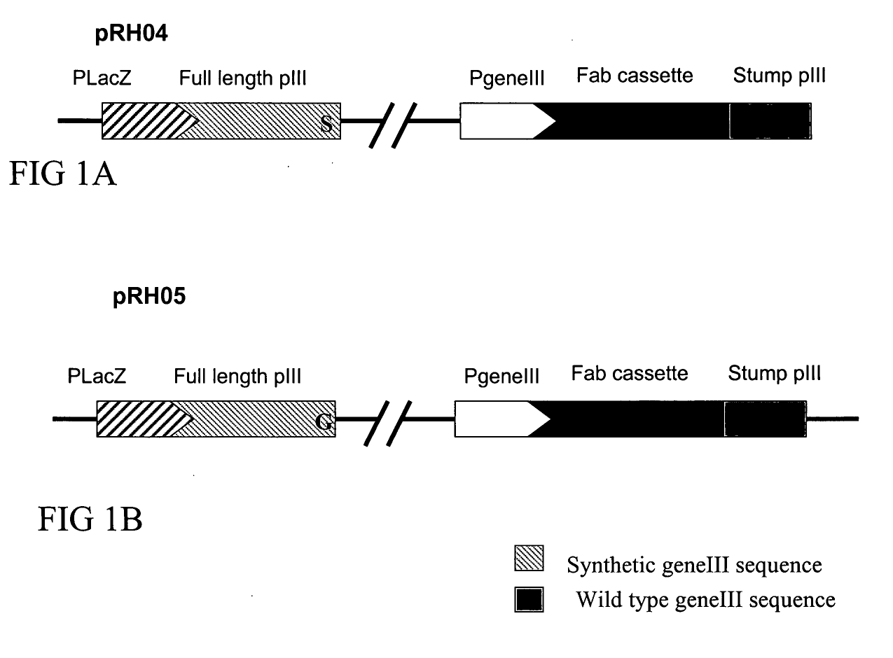

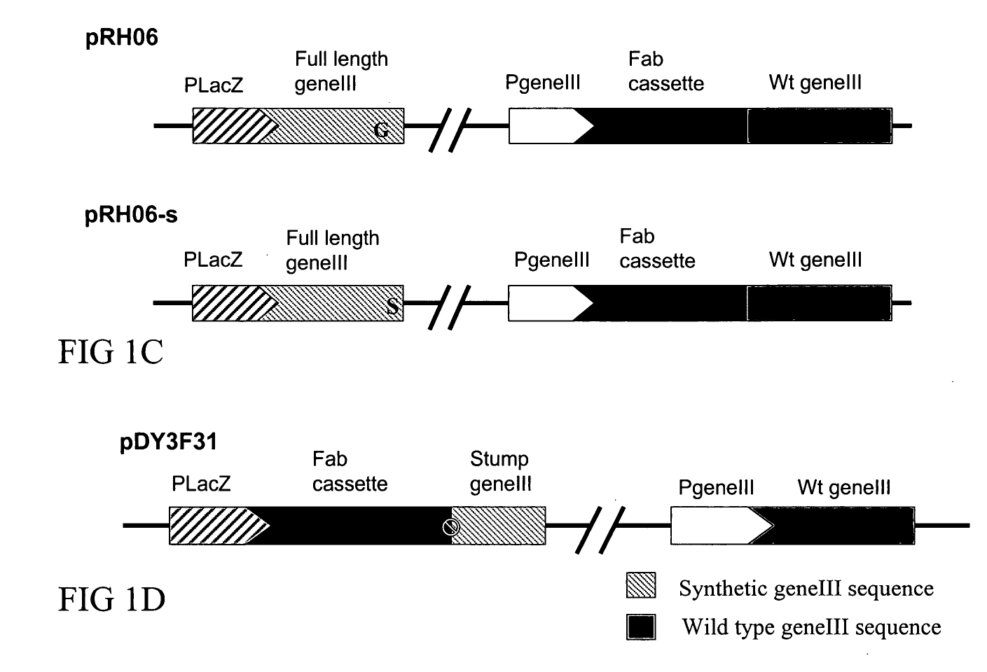

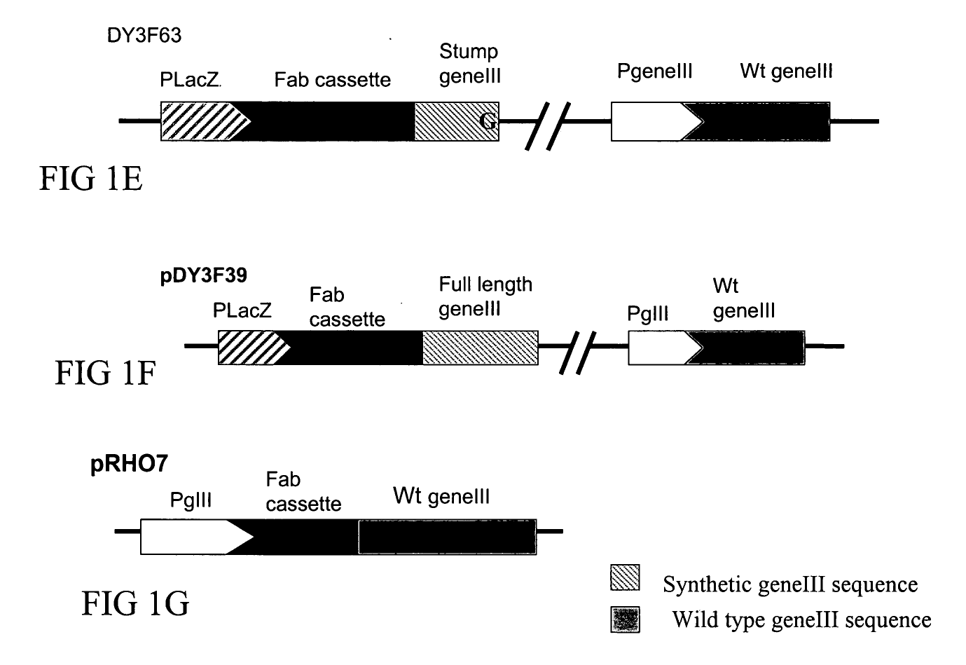

FIG. 1A is a schematic diagram of pRH04, a phage display vector in which the expression of the full-length gene III protein is regulated by a lac Z promoter, and expression of the Fab cassette/stump gene III fusion protein is regulated by gene III promoter Expression of the Fab cassette/stump gene III fusion from this vector is maximal. Expression of the full length gene III protein is regulatable.

When there is no glucose in the medium, there is only leaky expression of the full-length gene III protein. This allows for inclusion of multiple Fabs on the surface of the phage particles, a scenario suitable for selection based on avidity.

When there is IPTG in the medium, expression of the full length gene III protein is induced. Phages particles produced under these conditions have fewer Fab molecules per particle, a scenario suitable for selection based on affinity.

### EXAMPLE 2

### Determination of Antibody Display Efficiency of pRH04 and Comparison of pRH04 with DY3F31

D3 and E9 are two antibody fragments that bind to FITC (fluorescein isothiocyanate). Each of these antibody fragments was cloned into pRH04 and a second plasmid, DY3F31, using identical cloning sites. DY3F31 expresses the antibody fragment, under the control of a lac promoter, and the wild type gene III protein, under the control of the gene III promoter. This configuration of DY3F31 is the converse of pRH04. Thus, the valency of the invariant coat protein expressed by DY3F31 is not controlled in the same manner as is the invariant coat protein expressed by pRH04.

Phages were prepared using both pRH04 and DY3F31 as follows: Host cells containing DY3F31 were grown overnight at 37° C. in 2×TY medium+1 mM IPTG. Host cells containing pRH04 were grown overnight in 2×TY medium at 37° C.

Next, specific phage (D3-DY3F31 or D3-pRH04, or E9-DY3F31 or E9-pRH04) were produced and mixed with control fd-Tet-Dog1 phage, which do not bind FITC.

Immunotubes were coated with BSA-coupled FITC in 0.1 M carbonate buffer (pH=9.6) (50 μg/ml) and incubated for 90 min with different phage mixes in PBS-2% Marvel, washed ten times with PBS/Tween and two times with PBS, and eluted with 100 mM triethylamine.

After neutralization, a dilution series was made of the eluted phages and TG1 bacterial cells were added and incubated 30 min at 37° C. Dilutions were plated on agar plates containing either Ampicillin or Tetracyclin and grown overnight at 37° C. The next day the number of colonies on the plates were counted and the number of phage before selection (input) and the number of phage after selection (output) were determined.

The ratio between input and output phage is shown in Table 1 as well as the relative enrichment. Relative enrichment equals the recovery specific phage (E9 or D3) compared to background, as represented by a control phage (Fd-Tet-Dog1). No clear enrichment difference was observed between phage produced by the two phage vectors under these particular conditions.

TABLE 1
Results of the enrichment experiment comparing display
efficiency of DY3F31 and pRH04

| | Output/Input | | |
|---|---|---|---|
| | Recovery of | Recovery of fdTet | |
| Phage | specific phage | (control) phage | Enrichment |
| D3-DY3F31 | 3.5E−05 | 1.4E−05 | 2.5 |
| D3-pRH04 | 9.9E−05 | 5.9E−05 | 1.7 |
| E9-DY3F31 | 2.8E−04 | 4.9E−05 | 5.7 |
| E9-pRH04 | 7.5E−04 | 5.6E−05 | 13.4 |

In addition, ELISA was used to measure the relative quantity of antibody displayed on the phage of clone E9 in DY3F31 (E9-31) and E9 in pRH04 (E9-04, with and without 1 mM IPTG). In this ELISA, rabbit-anti-human kappa light chain antibody (Dako) was mixed with rabbit-anti-human lambda antibody (Dako) and coated for 16 h at 4° C. in 0.1 M carbonate buffer to an ELISA plate.

The next day, the plate was blocked for 1 h using 2% Marvel/PBS. Next, a dilution series of the different phages (with known titers) were cultured and incubated for 1 h with the blocked ELISA plate containing the anti-human kappa/lambda antibodies. After washing with PBS-Tween, anti-M13-HRP antibody, which binds the gene VIII protein present on all phage) was added. After incubation for 1 h, plates were washed with PBS-Tween and TMB substrate was added. The reaction was stopped after 5 min. with 2 M H₂SO₄ and OD₄₅₀ was measured.

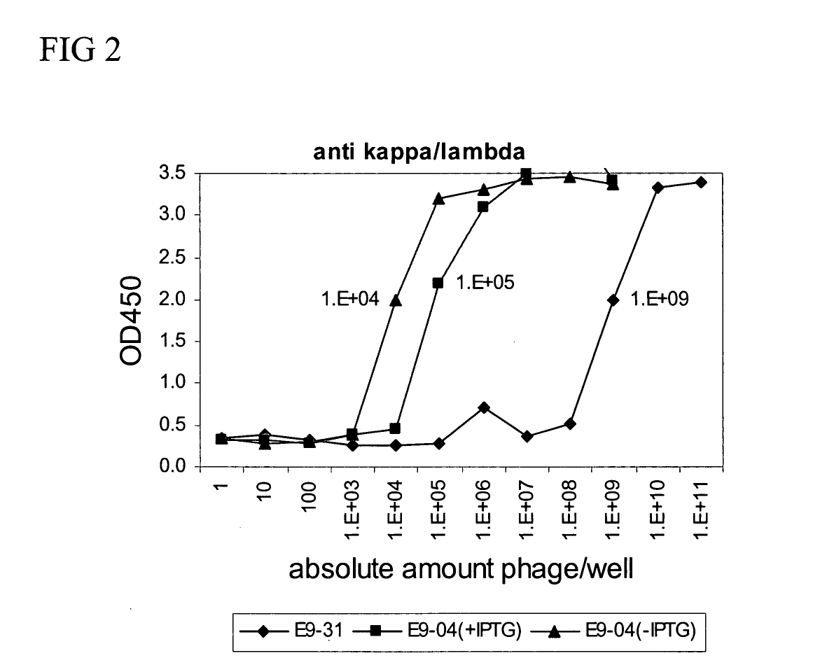

The results are depicted in FIG. 2. Phages containing pRH04 displayed a higher level of the antibody, because lower numbers of pRH04 phage displayed levels of antibody equivalent to the levels expressed on a far greater number of DY3F31 phage. FIG. 2 shows that 10⁴ (−IPTG) to 10⁵ (+IPTG) more phages are needed (based on titering) for DY3F31 to express equivalent levels of antibodies. The display of E9 by phage produced with pRH04 using identical number of infective phage particles is therefore 10⁴-10⁵ fold higher compared to DY3F31.

### EXAMPLE 3

### Construction of pRH05

DNA sequencing of pRH04 revealed a mutation in the synthetic gene III protein compared to the wild type gene III of bacteriophage M13. The nucleotide sequence was TCT at position 7745 instead of GGA, resulting in a serine to glycine change. To correct this mutation, a 179 base pair DNA fragment containing the DNA sequence at this position was generated by overlapping PCR. The PCR primers were designed to incorporate EcoRI and SacII restriction enzyme sites at the 5′ and 3′ ends of the fragment, respectively. The pRH04 phage vector and the fragment were digested with EcoRI and SacII and ligated to generate pRH05.

### EXAMPLE 4

### Determination of Functionality of pRH05

Antibody clone E9 directed to FITC was cloned from pRH04 into pRH05 using identical cloning sites as in pRH04. Phage were prepared from E9 in three different display systems; E9-DY3F31, E9-pRH04 and E9-RH05 using overnight growth at 37° C. in 2×TY+1 mM IPTG for DY3F31 and in 2×TY medium for pRH04 and pRH05.

Next, E9-DY3F31 or E9-pRH04 or E9-RH05 phages were mixed with control fd-Tet-Dog1 phage.

BSA-coupled FITC was coated to immunotubes (50 μg/ml) overnight in 0.1 M carbonate buffer (pH=9.6), blocked with 2% Marvel/PBS for 1 h, washed with PBS/Tween 20 and incubated for 90 min with different phage mixes in PBS-2% Marvel, subsequently washed ten times with PBS/Tween, two times with PBS, and eluted 10 min. with 100 mM triethylamine.

After neutralization, a dilution series was made of the eluted phages and TG1 bacterial cells were added and incubated 30 min at 37° C. Dilutions were plated on agar plates containing either Ampicillin or Tetracyclin and grown overnight at 37° C.

The next day, the number of colonies on the plates were counted and the number of phage before selection (input) and the number of phage after selection (output) were determined.

The ratio between input and output phage is shown in Table 2 as well as the relative enrichment (=the recovery specific phage (E9) over background non-relevant phage (Fd-Tet-Dog1). pRH05 showed 100 fold greater enrichment than pRH04 and pDY3F31.

TABLE 2
Enrichment of pRH05.

| Clone name | Output/Input | Output/Input fdTet | Enrichment |
|---|---|---|---|
| E9-DY3F31 | 4.0E−5 | 1.3E−5 | 3.1 |
| E9-pRH04 | 1.6E−3 | 4.2E−5 | 38 |
| E9-pRH05 | 2.5E−3 | 7.6E−6 | 329 |

ELISA was used to measure the relative quantity of antibody displayed on the phage for an antibody repertoire in DY3F31 (CJ-DY3F31), in pRH05 (kappa-pRH05) and pCES1 (CJ-pCES1). The nucleotide sequence of pCES1 is shown in Table 7 (see below). In this ELISA, rabbit-anti-human kappa light chain antibody (Dako) was mixed with rabbit-anti-human lambda antibody and coated to an ELISA plate for 16 h at 4° C. in 0.1 M carbonate buffer.

The next day, the plate was blocked for 1 h using 2% Marvel/PBS.

Subsequently, a dilution series of the different phages (with known titres) were made and incubated for 1 h. with the blocked ELISA plate containing the anti-human kappa/lambda antibodies. After washing with PBS-TWEEN, anti-M13-HRP antibody was added (directed to the gene VIII protein present on every phage). After incubation for 1 h, PBS-Tween washing was performed and TMB substrate was added. The reaction was stopped after 5 min. with 2 M H₂SO₄ and OD₄₅₀ was measured.

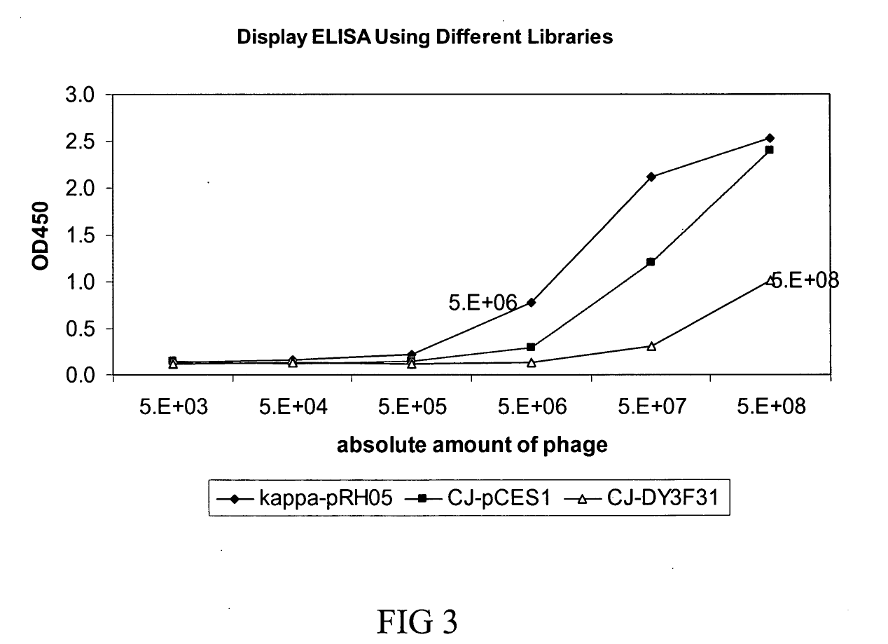

The display level of antibody repertoires (libraries) displayed by phage containing pRH05 (kappa-pRH05), pCES1 (CJ-pCES1) and DY3F31 (CJ-DY3F31) is shown in FIG. 3. pRH05 shows 5 fold greater display than pCes1 and 100 fold greater display than pDY3F31 phage.

### EXAMPLE 5

### Construction of pRH06

To increase the phage infectivity of multivalent displaying Fab of pRH05 the pRH06 vector was constructed. This vector contains two copies of full length gene III that are infective and allows regulation of the valency of the displayed polypeptide (Fab) on a phage display vector by up- or down- regulating the LacZ promoter that controls expression of the synthetic full length gene III protein. The expression of the Fab cassette/full length wild type gene III fusion protein is regulated by the gene III promoter (see schematic map of pRH06 in FIG. 1C).

To construct the pRH06 vector, 6 μg of pRH05 RF isolated DNA was digested for 2 h with 10 U/μg of SacI followed by heat inactivation of the enzyme and gel purification. 3 μg of the SacI linear pRH05 DNA was then digested for 2 h with AfeI (10 U/μg) followed by heat inactivation of the enzyme and gel purification in order to isolate the pRH05 backbone from the removed wild type gene III stump.

In parallel, the wild type gene III fragment was PCR amplified from DY3F31 for 25 cycles using a high fidelity thermostable polymerase, with a forward primer that anneals to the 5′ end of the wild type gene III containing a SacI restriction site at 5′ end (5′-GTCGTATGAGCTCTGCTGAAACTGTTGAAAGTTG-3′; SEQ ID NO:1), and a reverse primer that anneals within gene VI (5′-CTGAACACCCTGAACAAAGTC-3′; SEQ ID NO:2). After the PCR, the fragment was purified and 1.3 μg was digested for 2 h with 10 U/μg of SacI restriction enzyme followed by heat inactivation of the enzyme and purification. The PCR fragment was then digested overnight with 10 U/μg AfeI restriction enzyme followed by heat inactivation of the enzyme and gel purification of the fragment.

Ligation was performed for 2 h at room temperature using 63 ng wild type gene III PCR amplified fragment, 100 ng pRH05 backbone, and T4 DNA ligase. 25 ng of this ligation mixture was used in electroporation (1.7 kV;25 μF;200Ω) into E. coli XLI blue MRF′ cells (Stratagene).

To ensure a proper insertion of the wild type gene III in the pRH05 backbone, control PCR using specific wild type gene III primers and DNA sequencing were performed. The sequence of the pRH06 vector is shown below in Table 9.

### EXAMPLE 6

### Determination of Fab Display Efficiency of pRH06 and Comparison with pRH05

The D3 antibody fragment, which is directed to FITC (fluorescein isothiocyanate), was cloned into pRH06 and pRH05 using identical cloning sites. ELISA was used to measure the relative quantity of Fab displayed on the phage of clone D3 in pRH05 and pRH06 (with or without 2% glucose and with 1 mM IPTG). In this ELISA, rabbit-anti-human kappa light chain antibody (Dako) was mixed with rabbit-anti-human lambda antibody (Dako) and coated to an ELISA plate for 16 h at 4° C. in 0.1 M carbonate buffer. The next day, the plate was blocked for 1 h using 2% Marvel/PBS.

Next, 10¹⁰ phages were added and incubated for 1 h with the blocked ELISA plate containing the anti-human kappa/lambda antibodies. After washing with PBS-TWEEN (0.05%), anti-M13-HRP antibody, which is directed to the gene VIII protein present on every phage particle (Amersham 1:5000 diluted), was added. After incubation for 1 h, plates were washed with PBS-Tween (0.05%) and TMB substrate was added. The reaction was stopped after 5 min. with 2 M H₂SO₄ and OD₄₅₀ was measured.

### EXAMPLE 7

### Selection Using an Antibody Repertoire Cloned in pRH06

An antibody repertoire is cloned in pRH06 using identical cloning sites as in pRH04 and pRH05. For a schematic illustration of pRH06, see FIG. 1C. Phage is made overnight in 2×TY+2% glucose (conditions that allow high valency of Fab). This phage is used to select on immunotubes coated with BSA-coupled FITC (50 μg/ml) overnight in 0.1 M carbonate buffer (pH=9.6), blocked with 2% Marvel/PBS for 1 h, washed with PBS/Tween 20 and incubated for 90 min with the phage in PBS-2% Marvel, subsequently washed 10 times with PBS/Tween, 2 times with PBS, and eluted for 10 minutes with 100 mM triethylamine.

After neutralization, the eluted phages are used to infect TG1 cells and incubated 30 min at 37° C. and plated on agar plates containing 2×TY+Ampicillin+1 mM IPTG without the presence of glucose overnight at 30° C. The next day, plates are scraped, and bacteria are grown for an additional three hours starting at OD600=0.5 in 2×TY+IPTG at 37° C. (Phages with low valency). Next, phages are isolated by classical PEG precipitations and used to perform an additional selection on FITC-BSA. Therefore immunotubes coated with BSA-coupled FITC (50 μg/ml) overnight in 0.1 M carbonate buffer (pH=9.6) are used, blocked with 2% Marvel/PBS for 1 h, washed with PBS/Tween 20 and incubated for 90 min with the phage in PBS-2% Marvel, subsequently washed 10 times with PBS/Tween, 2 times with PBS, and eluted 10 min. with 100 mM triethylamine. After neutralization, the eluted phages are used to infect TG1 cells and incubated 30 min at 37° C. and plated on agar plates containing 2×TY+Ampicillin+2% Glucose overnight at 37° C. The next day, individual colonies are picked, grown in 2×TY+2% glucose and analyzed for binding to FITC-BSA in ELISA.

### EXAMPLE 8

### Construction of pRH06-S

To promote the incorporation of the Fab gene III fusion into the phage (e.g., to increase the Fab display) pRH06-Swas constructed. To do this, the S mutation in pRH04 (described above in Examples 3 and 4) was introduced into the full-length synthetic gene III (see FIG. 1C).

This mutation was found to decrease the incorporation of the synthetic gene III into the phage particle in pRH04 compared to pRH05 (see Example 4). Introduction of the mutation in pRH06-S was expected to favor the incorporation of the Fab wild type gene III versus the competing synthetic geneIII(S).

To construct pRH06-Sa 214 base pair fragment containing the serine mutation was generated from pRH04 vector via PCR using advantage 2 polymerase (25 cycles). The 5′ forward primer used contains the EcoRI restriction site (5′-CGAATTCTCAGATGGCCCAGGT-3′; SEQ ID NO:3) and the reverse 3′ primer contains the SacII restriction site (5′-GAAAACGCCGCGGAAAAGATTG-3′; SEQ ID NO:4). 4 μg of pRH06 was digested 3 hours with 20 U/μg SacII followed by gel purification. EcoRI digestion (20 U/μg, 3 hours) was performed, followed by gel purification.

The serine mutated fragment was digested the same way and gel purified.

Next, 25 ng cleaved and gel purified pRH06 vector was ligated with 40 ng insert (16° C. overnight) using T4 DNA ligase. The ligation-mixture was then transformed into E. coli TGI cells and the DNA sequence of the clones was determined the replacement of the TCT instead of GGA in the pRH06-S was confirmed, resulting in a serine to glycine change.

The sequence of the pRH06-Svector is shown in Table 10 (see below).

### EXAMPLE 9

### Determination of Functionality of pRH06-S

The Fab clone E9, which is directed to FITC, was cloned from pRH06 into pRH06-S using ApaL1 and NotI cloning sites. Phages were prepared from E9 in two different display systems; E9-pRH05 and E9-pRH06-Susing overnight growth at 30° C. (with 2% glucose, or without 2% glucose and with 1 mM IPTG).

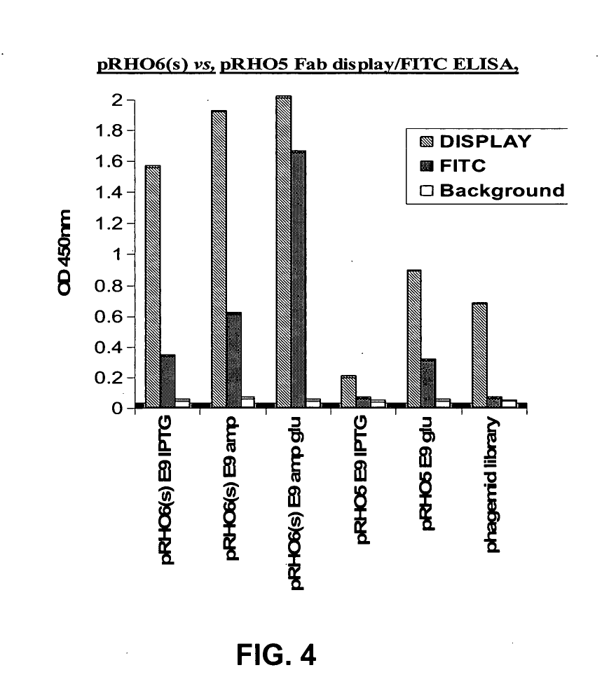

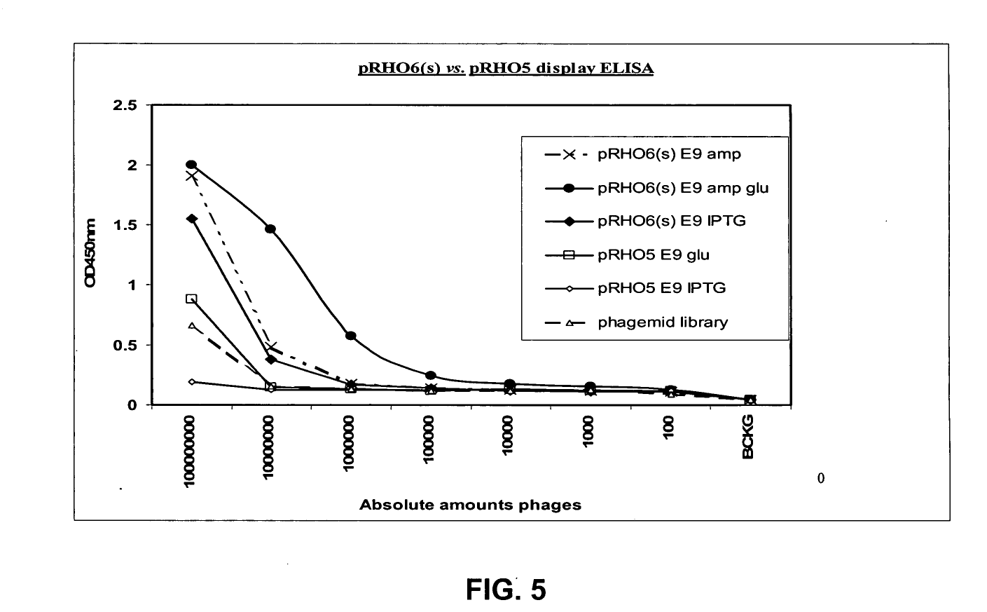

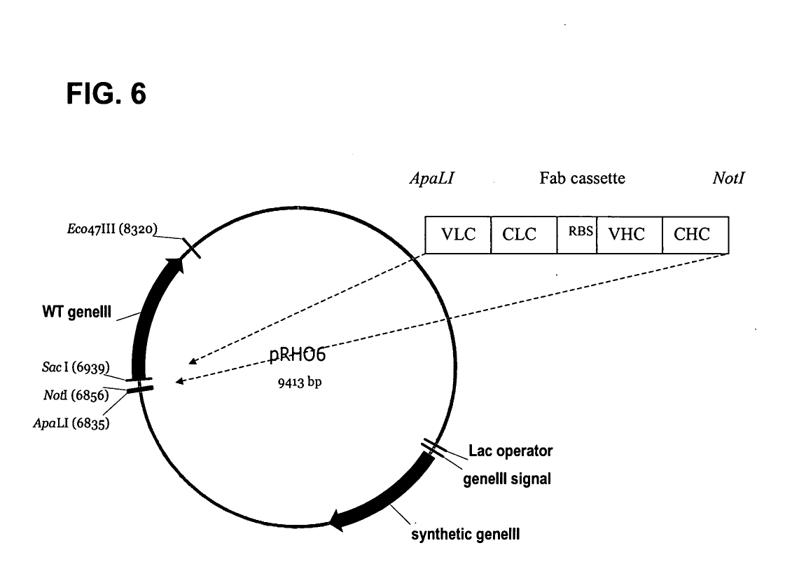

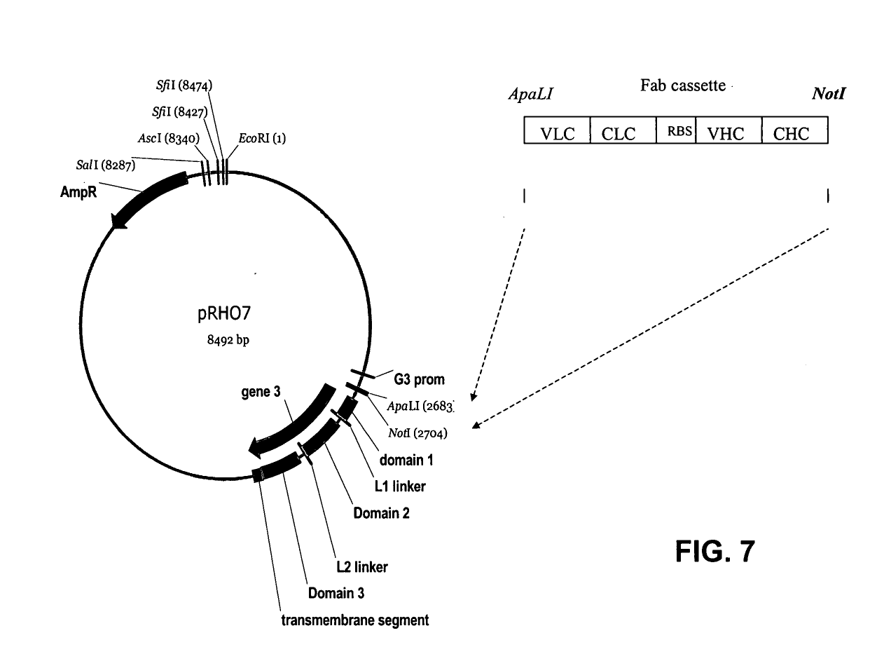

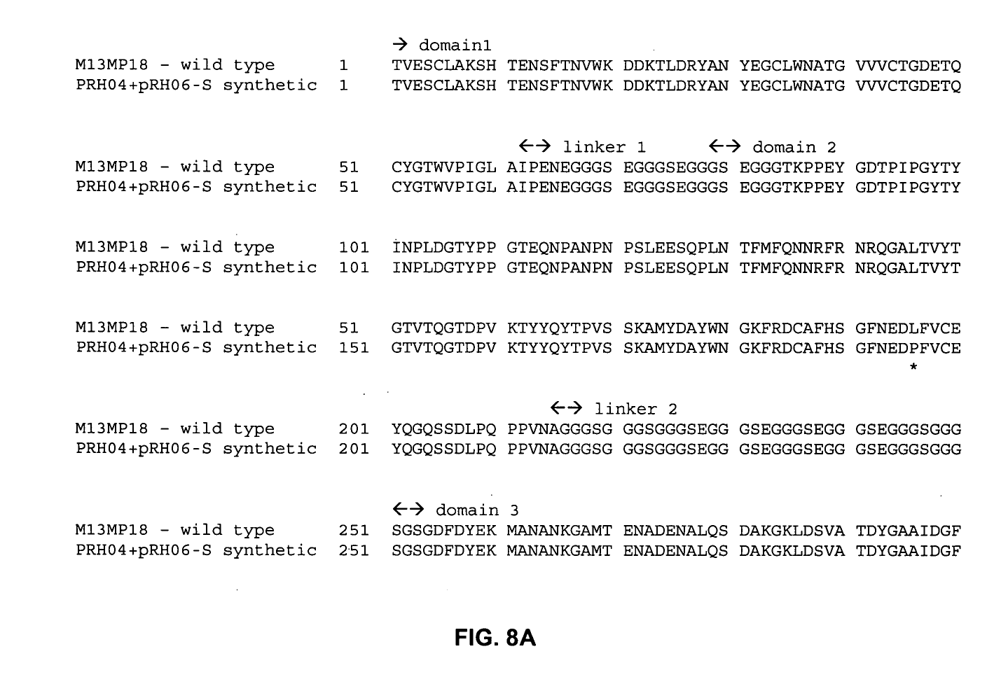

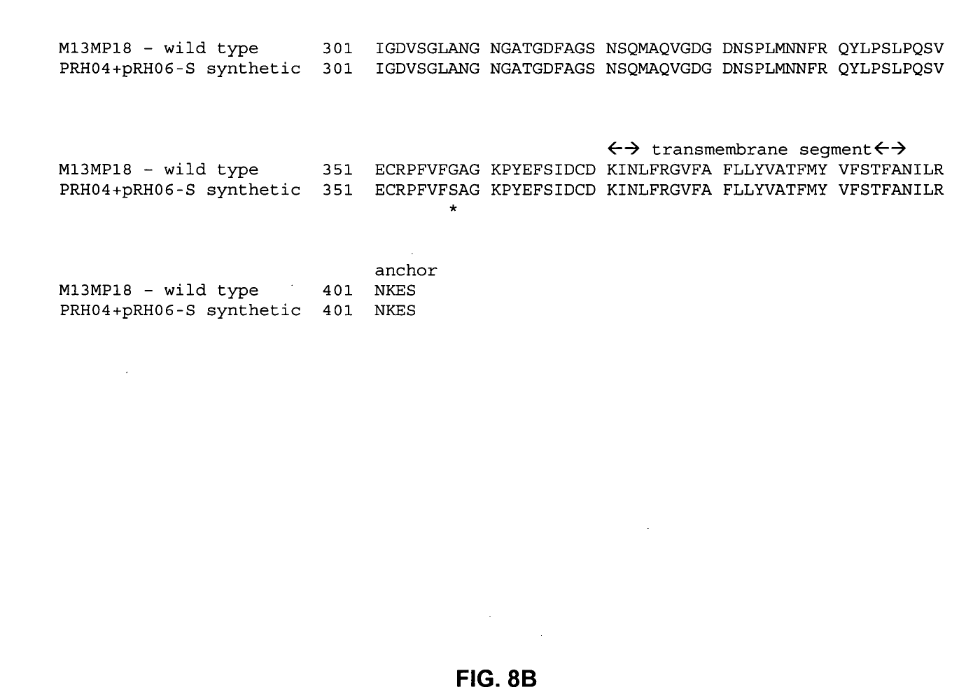

10⁸ phages were then used for display ELISA using the procedure described in Example 7. In parallel, a specific FITC ELISA was done using FITC-BSA (5 μg/ml in PBS) that had been coated on ELISA plates overnight 4° C. The next day, plates were blocked for 1 h using 2% Marvel/PBS and 1E8 phages were added and incubated for 1 h. After washing with PBS-Tween, anti-M13-HRP antibody (Amersham) was added. After incubation for 1 h, plates were washed with PBS-Tween and TMB substrate was added. The reaction was stopped after 5 min with 2 M H₂SO₄ and OD₄₅₀ was measured. The results are shown in FIG. 4, FIG. 5, and Table 12.

Using identical amounts of phage in this assay and using different culture conditions (+2% glucose; repression of the synthetic gene III expression, or induction of the synthetic gene III using 1 mM IPTG) a clear effect on the Fab display and binding to FITC is observed.

The highest Fab display and binding can be seen by repression of the Lac Z promoter using 2% glucose. Induction of the LacZ promoter with 1 M IPTG decreases the Fab display level and binding to FITC. The E9-FITC pRH06-Sshows about 1.5-2 times higher Fab display level in this assay than E9-FITC in pRH05 and 3 times higher than the Fab display of the phagemid library sample.

Two western blots were performed in parallel using the identical phage preparations. Detection was performed using the 9E10 antibody (directed to the c-myc tag present on the c-terminus of the heavy chain). A western blot that is probed with an anti-gene III antibody (MOBITEC) allowed detection of protein III and the Fab-PIII heavy chain fusion protein. This allows estimation of the copy number of Fab on the phage.

10⁸ phages from pRH06-S grown with 2×YT 100 μg/ml ampicillin and 1 mM IPTG; 10⁸ E9 pRHO6-S phage grown with 2×YT medium and 100 μg/ml ampicillin and finally 5×10⁷ phages from E9 pRHO6-S grown with 2×YT medium, 100 μg/ml ampicillin and 2% glucose.

These phage were denatured for 5 min at 85° C. in SDS loading buffer containing DTT then loaded on 4-10% SDS-PAGE gel and blotted on nitrocellulose membrane. After blotting, the membranes were blocked 1 hour in 4% Marvell PBS and 1/3000× diluted Anti-gene III protein monoclonal antibody (MOBITEC) was added as in parallel 9e10 anti c-Myc 1/1000 (DAKO). After one hour of incubation, the membranes were washed 5 times with PBS 0.1% TWEEN and 1 times with PBS. Next, rabbit anti mouse HRP (horse radish peroxidase) was added (1/1000 diluted in Marvel PBS/TWEEN). After one hour of incubation, the membrane were washed 5 times with PBS 0.1% TWEEN and once with PBS and ECL™ staining was performed.

An increase of gene III fusion protein (MW approx. 90 kD) was observed in phage prepared in 2×TYA with 2% glucose (repression of the LacZ promoter) compared to the same system grown using 2×TYA containing 1 mM IPTG (induction of LacZ) or 2×TYA only (no repression of LacZ). These experiments also confirmed that the valency of Fab display is increased by repression of the synthetic gene III in pRH06-S.

The relative level of Fab-gene III compared to the synthetic gene III (no fusion) is estimated to be 10%. The average number of gene III protein copies is 5 per phage particle. Thus, the Fab display level in pRH06-S is, on average, 0.5.

### EXAMPLE 10

### Construction of pRH07

pRH07 is a phage display vector containing the Fab cassette linked to a single copy of the wild type gene III regulated by the natural pIII promoter of gene III. A schematic representation of this vector in shown in FIG. 1G. The sequence is provided in Table 11. This vector allows display of multiple copies of Fab on the surface of phage.

To construct pRH07, 10 μg of pRH06 was digested 3 for h with 20 U/μg SalI, followed by heat inactivation of the enzyme and gel purification. A second restriction digestion was done using EcoRI, followed by heat inactivation of the enzyme, and gel purification of the vector backbone.

In parallel, a 222 bp stuffer, which does not contain gene III sequences, was created by PCR on DY3F31 and digested using EcoRI and SalI. The stuffer was ligated into the vector backbone to create the pRH07. The sequence of pRH07 is shown in Table.11. Proper construction was confirmed by DNA sequencing.

Table 3. pRH04 Nucleotide Sequence

Coding sequences are found beginning at or near these approximate nucleotide (nt) positions in pRH04 (5′ end-3′ end)

| | |
|---|---|
| Gene X: 496-831; Gene V: 843-1206 | |
| Gene VII: 1108-1206; Gene IX 1206-1304 | |
| Gene VII: 1301 | |
| Gene VIII: 1370 | |
| Gene III: 1579-2199 | |
| Gene VI: 2202-2540 | |
| b1a gene: 5491 | |
| Gene III: 6664 | |
| Gene III: 8283-831 | |
| AATGCTACTACTATTAGTAGAATTGATGCCACCTTTTCAGCTCGCGCCCCAAATGAAAATATAGCTAAACAGGTTAT | (SEQ ID NO:5) |
| TGACCATTTGCGAAATGTATCTAATGGTCAAACTAAATCTACTCGTTCGCAGAATTGGGAATCAACTGTTACATGGA | |
| ATGAAACTTCCAGACACCGTACTTTAGTTGCATATTTAAAACATGTTGAGCTACAGCACCAGATTCAGCAATTAAGC | |
| TCTAAGCCATCCGCAAAAATGACCTCTTATCAAAAGGAGCAATTAAAGGTACTCTCTAATCCTGACCTGTTGGAGTT | |
| TGCTTCCGGTCTGGTTCGCTTTGAAGCTCGAATTAAAACGCGATATTTGAAGTCTTTCGGGCTTCCTCTTAATCTTT | |
| TTGATGCAATCCGCTTTGCTTCTGACTATAATAGTCAGGGTAAAGACCTGATTTTTGATTTATGGTCATTCTCGTTT | |
| TCTGAACTGTTTAAAGCATTTGAGGGGGATTCAATGAATATTTATGACGATTCCGCAGTATTGGACGCTATCCAGTC | |
| TAAACATTTTACTATTACCCCCTCTGGCAAAACTTCTTTTGCAAAAGCCTCTCGCTATTTTGGTTTTTATCGTCGTC | |
| TGGTAAACGAGGGTTATGATAGTGTTGCTCTTACTATGCCTCGTAATTCCTTTTGGCGTTATGTATCTGCATTAGTT | |
| GAATGTGGTATTCCTAAATCTCAACTGATGAATCTTTCTACCTGTAATAATGTTGTTCCGTTAGTTCGTTTTATTAA | |
| CGTAGATTTTTCTTCCCAACGTCCTGACTGGTATAATGAGCCAGTTCTTAAAATCGCATAAGGTAATTCACAATGAT | |
| TAAAGTTGAAATTAAACCATCTCAAGCCCAATTTACTACTCGTTCTGGTGTTTCTCGTCAGGGCAAGCCTTATTCAC | |
| TGAATGAGCAGCTTTGTTACGTTGATTTGGGTAATGAATATCCGGTTCTTGTCAAGATTACTCTTGATGAAGGTCAG | |
| CCAGCCTATGCGCCTGGTCTGTACACCGTTCATCTGTCCTCTTTCAAAGTTGGTCAGTTCGGTTCCCTTATGATTGA | |
| CCGTCTGCGCCTCGTTCCGGCTAAGTAACATGGAGCAGGTCGCGGATTTCGACACAATTTATCAGGCGATGATACAA | |
| ATCTCCGTTGTACTTTGTTTCGCGCTTGGTATAATCGCTGGGGGTCAAAGATGAGTGTTTTAGTGTATTCTTTCGCC | |
| TCTTTCGTTTTAGGTTGGTGCCTTCGTAGTGGCATTACGTATTTTACCCGTTTAATGGAAACTTCCTCATGAAAAAG | |
| TCTTTAGTCCTCAAAGCCTCTGTAGCCGTTGCTACCCTCGTTCCGATGCTGTCTTTCGCTGCTGAGGGTGACGATCC | |
| CGCAAAAGCGGCCTTTAACTCCCTGCAAGCCTCAGCGACCGAATATATCGGTTATGCGTGGGCGATGGTTGTTGTCA | |
| TTGTCGGCGCAACTATCGGTATCAAGCTGTTTAAGAAATTCACCTCGAAAGCAAGCTGATAAACCGATACAATTAAA | |
| GGCTCCTTTTGGAGCCTTTTTTTTTGGAGATTTTCAACGTGAAAAAATTATTATTCGCAATTCCTTTAGTTGTTCCT | |
| TTCTATTCTCACAGTGCACAATCACATCTAGACGCGGCCGCTCATCACCACCATCATCACTCTGCTGAACAAAAACT | |
| CATCTCAGAAGAGGATCTGAATGGTGCCGCACAAGCGAGCTCTGCTTCCGGTGATTTTGATTATGAAAAGATGGCAA | |
| ACGCTAATAAGGGGGCTATGACCGAAAATGCCGATGAAAACGCGCTACAGTCTGACGCTAAAGGCAAACTTGATTCT | |
| GTCGCTACTGATTACGGTGCTGCTATCGATGGTTTCATTGGTGACGTTTCCGGCCTTGCTAATGGTAATGGTGCTAC | |
| TGGTGATTTTGCTGGCTCTAATTCCCAAATGGCTCAAGTCGGTGACGGTGATAATTCACCTTTAATGAATAATTTCC | |
| GTCAATATTTACCTTCCCTCCCTCAATCGGTTGAATGTCGCCCTTTTGTCTTTGGCGCTGGTAAACCATATGAATTT | |
| TCTATTGATTGTGACAAAATAAACTTATTCCGTGGTGTCTTTGCGTTTCTTTTATATGTTGCCACCTTTATGTATGT | |
| ATTTTCTACGTTTGCTAACATACTGCGTAATAAGGAGTCTTAATCATGCCAGTTCTTTTGGGTATTCCGTTATTATT | |
| GCGTTTCCTCGGTTTCCTTCTGGTAACTTTGTTCGGCTATCTGCTTACTTTTCTTAAAAAGGGCTTCGGTAAGATAG | |
| CTATTGCTATTTCATTGTTTCTTGCTCTTATTATTGGGCTTAACTCAATTCTTGTGGGTTATCTCTCTGATATTAGC | |
| GCTCAATTACCCTCTGACTTTGTTCAGGGTGTTCAGTTAATTCTCCCGTCTAATGCGCTTCCCTGTTTTTATGTTAT | |
| TCTCTCTGTAAAGGCTGCTATTTTCATTTTTGACGTTAAACAAAAAATCGTTTCTTATTTGGATTGGGATAAATAAT | |
| ATGGCTGTTTATTTTGTAACTGGCAAATTAGGCTCTGGAAAGACGCTCGTTAGCGTTGGTAAGATTCAGGATAAAAT | |
| TGTAGCTGGGTGCAAAATAGCAACTAATCTTGATTTAAGGCTTCAAAACCTCCCGCAAGTCGGGAGGTTCGCTAAAA | |
| CGCCTCGCGTTCTTAGAATACCGGATAAGCCTTCTATATCTGATTTGCTTGCTATTGGGCGCGGTAATGATTCCTAC | |
| GATGAAAATAAAAACGGCTTGCTTGTTCTCGATGAGTGCGGTACTTGGTTTAATACCCGTTCTTGGAATGATAAGGA | |
| AAGACAGCCGATTATTGATTGGTTTCTACATGCTCGTAAATTAGGATGGGATATTATTTTTCTTGTTCAGGACTTAT | |
| CTATTGTTGATAAACAGGCGCGTTCTGCATTAGCTGAACATGTTGTTTATTGTCGTCGTCTGGACAGAATTACTTTA | |
| CCTTTTGTCGGTACTTTATATTCTCTTATTACTGGCTCGAAAATGCCTCTGCCTAAATTACATGTTGGCGTTGTTAA | |
| ATATGGCGATTCTCAATTAAGCCCTACTGTTGAGCGTTGGCTTTATACTGGTAAGAATTTGTATAACGCATATGATA | |
| CTAAACAGGCTTTTTCTAGTAATTATGATTCCGGTGTTTATTCTTATTTAACGCCTTATTTATCACACGGTCGGTAT | |
| TTCAAACCATTAAATTTAGGTCAGAAGATGAAATTAACTAAAATATATTTGAAAAAGTTTTCTCGCGTTCTTTGTCT | |
| TGCGATTGGATTTGCATCAGCATTTACATATAGTTATATAACCCAACCTAAGCCGGAGGTTAAAAAGGTAGTCTCTC | |
| AGACCTATGATTTTGATAAATTCACTATTGACTCTTCTCAGCGTCTTAATCTAAGCTATCGCTATGTTTTCAAGGAT | |
| TCTAAGGGAAAATTAATTAATAGCGACGATTTACAGAAGCAAGGTTATTCACTCACATATATTGATTTATGTACTGT | |
| TTCCATTAAAAAAGGTAATTCAAATGAAATTGTTAAATGTAATTAATTTTGTTTTCTTGATGTTTGTTTCATCATCT | |
| TCTTTTGCTCAGGTAATTGAAATGAATAATTCGCCTCTGCGCGATTTTGTAACTTGGTATTCAAAGCAATCAGGCGA | |
| ATCCGTTATTGTTTCTCCCGATGTAAAAGGTACTGTTACTGTATATTCATCTGACGTTAAACCTGAAAATCTACGCA | |
| ATTTCTTTATTTCTGTTTTACGTGCAAATAATTTTGATATGGTAGGTTCTAACCCTTCCATTATTCAGAAGTATAAT | |
| CCAAACAATCAGGATTATATTGATGAATTGCCATCATCTGATAATCAGGAATATGATGATAATTCCGCTCCTTCTGG | |
| TGGTTTCTTTGTTCCGCAAAATGATAATGTTACTCAAACTTTTAAAATTAATAACGTTCGGGCAAAGGATTTAATAC | |
| GAGTTGTCGAATTGTTTGTAAAGTCTAATACTTCTAAATCCTCAAATGTATTATCTATTGACGGCTCTAATCTATTA | |
| GTTGTTAGTGCTCCTAAAGATATTTTAGATAACCTTCCTCAATTCCTTTCAACTGTTGATTTGCCAACTGACCAGAT | |
| ATTGATTGAGGGTTTGATATTTGAGGTTCAGCAAGGTGATGCTTTAGATTTTTCATTTGCTGCTGGCTCTCAGCGTG | |
| GCACTGTTGCAGGCGGTGTTAATACTGACCGCCTCACCTCTGTTTTATCTTCTGCTGGTGGTTCGTTCGGTATTTTT | |
| AATGGCGATGTTTTAGGGCTATCAGTTCGCGCATTAAAGACTAATAGCCATTCAAAAATATTGTCTGTGCCACGTAT | |
| TCTTACGCTTTCAGGTCAGAAGGGTTCTATCTCTGTTGGCCAGAATGTCCCTTTTATTACTGGTCGTGTGACTGGTG | |
| AATCTGCCAATGTAAATAATCCATTTCAGACGATTGAGCGTCAAAATGTAGGTATTTCCATGAGCGTTTTTCCTGTT | |
| GCAATGGCTGGCGGTAATATTGTTCTGGATATTACCAGCAAGGCCGATAGTTTGAGTTCTTCTACTCAGGCAAGTGA | |
| TGTTATTACTAATCAAAGAAGTATTGCTACAACGGTTAATTTGCGTGATGGACAGACTCTTTTACTCGGTGGCCTCA | |
| CTGATTATAAAAACACTTCTCAGGATTCTGGCGTACCGTTCCTGTCTAAAATCCCTTTAATCGGCCTCCTGTTTAGC | |
| TCCCGCTCTGATTCTAACGAGGAAAGCACGTTATACGTGCTCGTCAAAGCAACCATAGTACGCGCCCTGTAGCGGCG | |
| CATTAAGCGCGGCGGGTGTGGTGGTTACGCGCAGCGTGACCGCTACACTTGCCAGCGCCCTAGCGCCCGCTCCTTTC | |
| GCTTTCTTCCCTTCCTTTCTCGCCACGTTCGCCGGCTTTCCCCGTCAAGCTCTAAATCGGGGGCTCCCTTTAGGGTT | |
| CCGATTTAGTGCTTTACGGCACCTCGACCCCAAAAAACTTGATTTGGGTGATGGTTCACGTAGTGGGCCATCGCCCT | |
| GATAGACGGTTTTTCGCCCTTTGACGTTGGAGTCCACGTTCTTTAATAGTGGACTCTTGTTCCAAACTGGAACAACA | |
| CTCAACCCTATCTCGGGCTATTCTTTTGATTTATAAGGGATTTTGCCGATTTCGGAACCACCATCAAACAGGATTTT | |
| CGCCTGCTGGGGCAAACCAGCGTGGACCGCTTGCTGCAACTCTCTCAGGGCCAGGCGGTGAAGGGCAATCAGCTGTT | |
| GCCCGTCTCACTGGTGAAAAGAAAAACCACCCTGGATCCAAGCTTGCAGGTGGCACTTTTCGGGGAAATGTGCGCGG | |
| AACCCCTATTTGTTTATTTTTCTAAATACATTCAAATATGTATCCGCTCATGAGACAATAACCCTGATAAATGCTTC | |
| AATAATATTGAAAAAGGAAGAGTATGAGTATTCAACATTTCCGTGTCGCCCTTATTCCCTTTTTTGCGGCATTTTGC | |
| CTTCCTGTTTTTGCTCACCCAGAAACGCTGGTGAAAGTAAAAGATGCTGAAGATCAGTTGGGCGCACTAGTGGGTTA | |
| CATCGAACTGGATCTCAACAGCGGTAAGATCCTTGAGAGTTTTCGCCCCGAAGAACGTTTTCCAATGATGAGCACTT | |
| TTAAAGTTCTGCTATGTGGCGCGGTATTATCCCGTATTGACGCCGGGCAAGAGCAACTCGGTCGCCGCATACACTAT | |
| TCTCAGAATGACTTGGTTGAGTACTCACCAGTCACAGAAAAGCATCTTACGGATGGCATGACAGTAAGAGAATTATG | |
| CAGTGCTGCCATAACCATGAGTGATAACACTGCGGCCAACTTACTTCTGACAACGATCGGAGGACCGAAGGAGCTAA | |
| CCGCTTTTTTGCACAACATGGGGGATCATGTAACTCGCCTTGATCGTTGGGAACCGGAGCTGAATGAAGCCATACCA | |
| AACGACGAGCGTGACACCACGATGCCTGTAGCAATGGCAACAACGTTGCGCAAACTATTAACTGGCGAACTACTTAC | |
| TCTAGCTTCCCGGCAACAATTAATAGACTGGATGGAGGCGGATAAAGTTGCAGGACCACTTCTGCGCTCGGCCCTTC | |
| CGGCTGGCTGGTTTATTGCTGATAAATCTGGAGCCGGTGAGCGTGGGTCTCGCGGTATCATTGCAGCACTGGGGCCA | |
| GATGGTAAGCCCTCCCGTATCGTAGTTATCTACACGACGGGGAGTCAGGCAACTATGGATGAACGAAATAGACAGAT | |
| CGCTGAGATAGGTGCCTCACTGATTAAGCATTGGTAACTGTCAGACCAAGTTTACTCATATATACTTTAGATTGATT | |
| TAAAACTTCATTTTTAATTTAAAAGGATCTAGGTGAAGATCCTTTTTGATAATCTCATGACCAAAATCCCTTAACGT | |
| GAGTTTTCGTTCCACTGTACGTAAGACCCCCAAGCTTGTCGACCGCAACGCAATTAATGTGAGTTAGCTCACTCATT | |
| AGGCACCCCAGGCTTTACACTTTATGCTTCCGGCTCGTATGTTGTGTGGAATTGTGAGCGGATAACAATTTCACCCA | |
| TGCTTTGGACAGGAAACAGCTATGAAAAAGCTTTTATTCGCTATCCCGTTAGTTGTACCGTTCTATTCTCACTCTGC | |
| CGAGACAGTCGAATCCTGCCTGGCCAAGGTCCACACTGAGAATAGTTTCACAAATGTGTGGAAGGATGATAAGACCC | |
| TTGATCGATATGCCAATTACGAAGGCTGCTTATGGAATGCCACCGGCGTCGTTGTCTGCACGGGCGATGAGACACAA | |
| TGCTATGGCACGTGGGTGCCGATAGGCTTAGCCATACCGGAGAACGAAGGCGGCGGTAGCGAAGGCGGTGGCAGCGA | |
| AGGCGGTGGATCCGAAGGAGGTGGAACCAAGCCGCCGGAATATGGCGACACTCCGATACCTGGTTACACCTACATTA | |
| ATCCGTTAGATGGAACCTACCCTCCGGGCACCGAACAGAATCCTGCCAACCCGAACCCAAGCTTAGAAGAAAGCCAA | |
| CCGTTAAACACCTTTATGTTCCAAAACAACCGTTTTAGGAACCGTCAAGGTGCTCTTACCGTGTACACTGGAACCGT | |
| CACCCAGGGTACCGATCCTGTCAAGACCTACTATCAATATACCCCGGTCTCGAGTAAGGCTATGTACGATGCCTATT | |
| GGAATGGCAAGTTTCGTGATTGTGCCTTTCACAGCGGTTTCAACGAAGACCCTTTTGTCTGCGAGTACCAGGGTCAG | |
| AGTAGCGATTTACCGCAGCCACCGGTTAACGCGGGTGGTGGTAGCGGCGGAGGCAGCGGCGGTGGTAGCGAAGGCGG | |
| AGGTAGCGAAGGAGGTGGCAGCGGAGGCGGTAGCGGCAGTGGCGACTTCGACTACGAGAAAATGGCTAATGCCAACA | |
| AAGGCGCCATGACTGAGAACGCTGACGAGAATGCACTGCAAAGTGATGCCAAGGGTAAGTTAGACAGCGTCGCCACA | |
| GACTATGGTGCTGCCATCGACGGCTTTATCGGCGATGTCAGTGGTCTGGCTAACGGCAACGGAGCCACCGGAGACTT | |
| CGCAGGTTCGAATTCTCAGATGGCCCAGGTTGGAGATGGGGACAACAGTCCGCTTATGAACAACTTTAGACAGTACC | |
| TTCCGTCTCTTCCGCAGAGTGTCGAGTGCCGTCCATTCGTTTTCTCTGCCGGCAAGCCTTACGAGTTCAGCATCGAC | |
| TGCGATAAGATCAATCTTTTCCGCGGCGTTTTCGCTTTCTTGCTATACGTCGCTACTTTCATGTACGTTTTCAGCAC | |
| TTTCGCCAATATTTTACGCAACAAAGAAAGCTAGTGATCTCCTAGGAAGCCCGCCTAATGAGCGGGCTTTTTTTTTC | |
| TGGTATGCATCCTGAGGCCGATACTGTCGTCGTCCCCTCAAACTGGCAGATGCACGGTTACGATGCGCCCATCTACA | |
| CCAACGTGACCTATCCCATTACGGTCAATCCGCCGTTTGTTCCCACGGAGAATCCGACGGGTTGTTACTCGCTCACA | |
| TTTAATGTTGATGAAAGCTGGCTACAGGAAGGCCAGACGCGAATTATTTTTGATGGCGTTCCTATTGGTTAAAAAAT | |
| GAGCTGATTTAACAAAAATTTAATGCGAATTTTAACAAAATATTAACGTTTACAATTTAAATATTTGCTTATACAAT | |
| CTTCCTGTTTTTGGGGCTTTTCTGATTATCAACCGGGGTACATATGATTGACATGCTAGTTTTACGATTACCGTTCA | |
| TCGATTCTCTTGTTTGCTCCAGACTCTCAGGCAATGACCTGATAGCCTTTGTAGATCTCTCAAAAATAGCTACCCTC | |
| TCCGGCATGAATTTATCAGCTAGAACGGTTGAATATCATATTGATGGTGATTTGACTGTCTCCGGCCTTTCTCACCC | |
| TTTTGAATCTTTACCTACACATTACTCAGGCATTGCATTTAAAATATATGAGGGTTCTAAAAATTTTTATCCTTGCG | |
| TTGAAATAAAGGCTTCTCCCGCAAAAGTATTACAGGGTCATAATGTTTTTGGTACAACCGATTTAGCTTTATGCTCT | |
| GAGGCTTTATTGCTTAATTTTGCTAATTCTTTGCCTTGCCTGTATGATTTATTGGATGTT | |

TABLE 4
Malia2 nucleotide sequence.

| | |
|---|---|
| AATGCTACTACTATTAGTAGAATTGATGCCACCTTTTCAGCTCGCGCCCCAAATGAAAATATAGCTAAACAGGTTAT | (SEQ ID NO:6) |
| TGACCATTTGCGAAATGTATCTAATGGTCAAACTAAATCTACTCGTTCGCAGAATTGGGAATCAACTGTTACATGGA | |
| ATGAAACTTCCAGACACCGTACTTTAGTTGCATATTTAAAACATGTTGAGCTACAGCACCAGATTCAGCAATTAAGC | |
| TCTAAGCCATCCGCAAAAATGACCTCTTATCAAAAGGAGCAATTAAAGGTACTCTCTAATCCTGACCTGTTGGAGTT | |
| TGCTTCCGGTCTGGTTCGCTTTGAAGCTCGAATTAAAACGCGATATTTGAAGTCTTTCGGGCTTCCTCTTAATCTTT | |
| TTGATGCAATCCGCTTTGCTTCTGACTATAATAGTCAGGGTAAAGACCTGATTTTTGATTTATGGTCATTCTCGTTT | |
| TCTGAACTGTTTAAAGCATTTGAGGGGGATTCAATGAATATTTATGACGATTCCGCAGTATTGGACGCTATCCAGTC | |
| TAAACATTTTACTATTACCCCCTCTGGCAAAACTTCTTTTGCAAAAGCCTCTCGCTATTTTGGTTTTTATCGTCGTC | |
| TGGTAAACGAGGGTTATGATAGTGTTGCTCTTACTATGCCTCGTAATTCCTTTTGGCGTTATGTATCTGCATTAGTT | |
| GAATGTGGTATTCCTAAATCTCAACTGATGAATCTTTCTACCTGTAATAATGTTGTTCCGTTAGTTCGTTTTATTAA | |
| CGTAGATTTTTCTTCCCAACGTCCTGACTGGTATAATGAGCCAGTTCTTAAAATCGCATAAGGTAATTCACAATGAT | |
| TAAAGTTGAAATTAAACCATCTCAAGCCCAATTTACTACTCGTTCTGGTGTTTCTCGTCAGGGCAAGCCTTATTCAC | |
| TGAATGAGCAGCTTTGTTACGTTGATTTGGGTAATGAATATCCGGTTCTTGTCAAGATTACTCTTGATGAAGGTCAG | |
| CCAGCCTATGCGCCTGGTCTGTACACCGTTCATCTGTCCTCTTTCAAAGTTGGTCAGTTCGGTTCCCTTATGATTGA | |
| CCGTCTGCGCCTCGTTCCGGCTAAGTAACATGGAGCAGGTCGCGGATTTCGACACAATTTATCAGGCGATGATACAA | |
| ATCTCCGTTGTACTTTGTTTCGCGCTTGGTATAATCGCTGGGGGTCAAAGATGAGTGTTTTAGTGTATTCTTTCGCC | |
| TCTTTCGTTTTAGGTTGGTGCCTTCGTAGTGGCATTACGTATTTTACCCGTTTAATGGAAACTTCCTCATGAAAAAG | |
| TCTTTAGTCCTCAAAGCCTCTGTAGCCGTTGCTACCCTCGTTCCGATGCTGTCTTTCGCTGCTGAGGGTGACGATCC | |
| CGCAAAAGCGGCCTTTAACTCCCTGCAAGCCTCAGCGACCGAATATATCGGTTATGCGTGGGCGATGGTTGTTGTCA | |
| TTGTCGGCGCAACTATCGGTATCAAGCTGTTTAAGAAATTCACCTCGAAAGCAAGCTGATAAACCGATACAATTAAA | |
| GGCTCCTTTTGGAGCCTTTTTTTTTGGAGATTTTCAACGTGAAAAAATTATTATTCGCAATTCCTTTAGTTGTTCCT | |
| TTCTATTCTCACAGTGCACAATCACATCTAGACGCGGCCGCTCATCACCACCATCATCACTCTGCTGAACAAAAACT | |
| CATCTCAGAAGAGGATCTGAATGGTGCCGCAGATATCAACGATGATCGTATGGCTAGCGGCGCCGCTGAAACTGTTG | |
| AAAGTTGTTTAGCAAAACCCCATACAGAAAATTCATTTACTAACGTCTGGAAAGACGACAAAACTTTAGATCGTTAC | |
| GCTAACTATGAGGGTTGTCTGTGGAATGCTACAGGCGTTGTAGTTTGTACTGGTGACGAAACTCAGTGTTACGGTAC | |
| ATGGGTTCCTATTGGGCTTGCTATCCCTGAAAATGAGGGTGGTGGCTCTGAGGGTGGCGGTTCTGAGGGTGGCGGTT | |
| CTGAGGGTGGCGGTACTAAACCTCCTGAGTACGGTGATACACCTATTCCGGGCTATACTTATATCAACCCTCTCGAC | |
| GGCACTTATCCGCCTGGTACTGAGCAAAACCCCGCTAATCCTAATCCTTCTCTTGAGGAGTCTCAGCCTCTTAATAC | |
| TTTCATGTTTCAGAATAATAGGTTCCGAAATAGGCAGGGGGCATTAACTGTTTATACGGGCACTGTTACTCAAGGCA | |
| CTGACCCCGTTAAAACTTATTACCAGTACACTCCTGTATCATCAAAAGCCATGTATGACGCTTACTGGAACGGTAAA | |
| TTCAGAGACTGCGCTTTCCATTCTGGCTTTAATGAAGATCCATTCGTTTGTGAATATCAAGGCCAATCGTCTGACCT | |
| GCCTCAACCTCCTGTCAATGCTGGCGGCGGCTCTGGTGGTGGTTCTGGTGGCGGCTCTGAGGGTGGTGGCTCTGAGG | |
| GTGGCGGTTCTGAGGGTGGCGGCTCTGAGGGAGGCGGTTCCGGTGGTGGCTCTGGTTCCGGTGATTTTGATTATGAA | |
| AAGATGGCAAACGCTAATAAGGGGGCTATGACCGAAAATGCCGATGAAAACGCGCTACAGTCTGACGCTAAAGGCAA | |
| ACTTGATTCTGTCGCTACTGATTACGGTGCTGCTATCGATGGTTTCATTGGTGACGTTTCCGGCCTTGCTAATGGTA | |
| ATGGTGCTACTGGTGATTTTGCTGGCTCTAATTCCCAAATGGCTCAAGTCGGTGACGGTGATAATTCACCTTTAATG | |
| AATAATTTCCGTCAATATTTACCTTCCCTCCCTCAATCGGTTGAATGTCGCCCTTTTGTCTTTAGCGCTGGTAAACC | |
| ATATGAATTTTCTATTGATTGTGACAAAATAAACTTATTCCGTGGTGTCTTTGCGTTTCTTTTATATGTTGCCACCT | |
| TTATGTATGTATTTTCTACGTTTGCTAACATACTGCGTAATAAGGAGTCTTAATCATGCCAGTTCTTTTGGGTATTC | |
| CGTTATTATTGCGTTTCCTCGGTTTCCTTCTGGTAACTTTGTTCGGCTATCTGCTTACTTTTCTTAAAAAGGGCTTC | |
| GGTAAGATAGCTATTGCTATTTCATTGTTTCTTGCTCTTATTATTGGGCTTAACTCAATTCTTGTGGGTTATCTCTC | |
| TGATATTAGCGCTCAATTACCCTCTGACTTTGTTCAGGGTGTTCAGTTAATTCTCCCGTCTAATGCGCTTCCCTGTT | |
| TTTATGTTATTCTCTCTGTAAAGGCTGCTATTTTCATTTTTGACGTTAAACAAAAAATCGTTTCTTATTTGGATTGG | |
| GATAAATAATATGGCTGTTTATTTTGTAACTGGCAAATTAGGCTCTGGAAAGACGCTCGTTAGCGTTGGTAAGATTC | |
| AGGATAAAATTGTAGCTGGGTGCAAAATAGCAACTAATCTTGATTTAAGGCTTCAAAACCTCCCGCAAGTCGGGAGG | |
| TTCGCTAAAACGCCTCGCGTTCTTAGAATACCGGATAAGCCTTCTATATCTGATTTGCTTGCTATTGGGCGCGGTAA | |
| TGATTCCTACGATGAAAATAAAAACGGCTTGCTTGTTCTCGATGAGTGCGGTACTTGGTTTAATACCCGTTCTTGGA | |
| ATGATAAGGAAAGACAGCCGATTATTGATTGGTTTCTACATGCTCGTAAATTAGGATGGGATATTATTTTTCTTGTT | |
| CAGGACTTATCTATTGTTGATAAACAGGCGCGTTCTGCATTAGCTGAACATGTTGTTTATTGTCGTCGTCTGGACAG | |
| AATTACTTTACCTTTTGTCGGTACTTTATATTCTCTTATTACTGGCTCGAAAATGCCTCTGCCTAAATTACATGTTG | |
| GCGTTGTTAAATATGGCGATTCTCAATTAAGCCCTACTGTTGAGCGTTGGCTTTATACTGGTAAGAATTTGTATAAC | |
| GCATATGATACTAAACAGGCTTTTTCTAGTAATTATGATTCCGGTGTTTATTCTTATTTAACGCCTTATTTATCACA | |
| CGGTCGGTATTTCAAACCATTAAATTTAGGTCAGAAGATGAAATTAACTAAAATATATTTGAAAAAGTTTTCTCGCG | |
| TTCTTTGTCTTGCGATTGGATTTGCATCAGCATTTACATATAGTTATATAACCCAACCTAAGCCGGAGGTTAAAAAG | |
| GTAGTCTCTCAGACCTATGATTTTGATAAATTCACTATTGACTCTTCTCAGCGTCTTAATCTAAGCTATCGCTATGT | |
| TTTCAAGGATTCTAAGGGAAAATTAATTAATAGCGACGATTTACAGAAGCAAGGTTATTCACTCACATATATTGATT | |
| TATGTACTGTTTCCATTAAAAAAGGTAATTCAAATGAAATTGTTAAATGTAATTAATTTTGTTTTCTTGATGTTTGT | |
| TTCATCATCTTCTTTTGCTCAGGTAATTGAAATGAATAATTCGCCTCTGCGCGATTTTGTAACTTGGTATTCAAAGC | |
| AATCAGGCGAATCCGTTATTGTTTCTCCCGATGTAAAAGGTACTGTTACTGTATATTCATCTGACGTTAAACCTGAA | |
| AATCTACGCAATTTCTTTATTTCTGTTTTACGTGCTAATAATTTTGATATGGTTGGTTCAATTCCTTCCATAATTCA | |
| GAAGTATAATCCAAACAATCAGGATTATATTGATGAATTGCCATCATCTGATAATCAGGAATATGATGATAATTCCG | |
| CTCCTTCTGGTGGTTTCTTTGTTCCGCAAAATGATAATGTTACTCAAACTTTTAAAATTAATAACGTTCGGGCAAAG | |
| GATTTAATACGAGTTGTCGAATTGTTTGTAAAGTCTAATACTTCTAAATCCTCAAATGTATTATCTATTGACGGCTC | |
| TAATCTATTAGTTGTTTCTGCACCTAAAGATATTTTAGATAACCTTCCTCAATTCCTTTCTACTGTTGATTTGCCAA | |
| CTGACCAGATATTGATTGAGGGTTTGATATTTGAGGTTCAGCAAGGTGATGCTTTAGATTTTTCATTTGCTGCTGGC | |
| TCTCAGCGTGGCACTGTTGCAGGCGGTGTTAATACTGACCGCCTCACCTCTGTTTTATCTTCTGCTGGTGGTTCGTT | |
| CGGTATTTTTAATGGCGATGTTTTAGGGCTATCAGTTCGCGCATTAAAGACTAATAGCCATTCAAAAATATTGTCTG | |
| TGCCACGTATTCTTACGCTTTCAGGTCAGAAGGGTTCTATCTCTGTTGGCCAGAATGTCCCTTTTATTACTGGTCGT | |
| GTGACTGGTGAATCTGCCAATGTAAATAATCCATTTCAGACGATTGAGCGTCAAAATGTAGGTATTTCCATGAGCGT | |
| TTTTCCTGTTGCAATGGCTGGCGGTAATATTGTTCTGGATATTACCAGCAAGGCCGATAGTTTGAGTTCTTCTACTC | |
| AGGCAAGTGATGTTATTACTAATCAAAGAAGTATTGCTACAACGGTTAATTTGCGTGATGGACAGACTCTTTTACTC | |
| GGTGGCCTCACTGATTATAAAAACACTTCTCAAGATTCTGGCGTACCGTTCCTGTCTAAAATCCCTTTAATCGGCCT | |
| CCTGTTTAGCTCCCGCTCTGATTCCAACGAGGAAAGCACGTTATACGTGCTCGTCAAAGCAACCATAGTACGCGCCC | |
| TGTAGCGGCGCATTAAGCGCGGCGGGTGTGGTGGTTACGCGCAGCGTGACCGCTACACTTGCCAGCGCCCTAGCGCC | |
| CGCTCCTTTCGCTTTCTTCCCTTCCTTTCTCGCCACGTTCGCCGGCTTTCCCCGTCAAGCTCTAAATCGGGGGCTCC | |
| CTTTAGGGTTCCGATTTAGTGCTTTACGGCACCTCGACCCCAAAAAACTTGATTTGGGTGATGGTTCACGTAGTGGG | |
| CCATCGCCCTGATAGACGGTTTTTCGCCCTTTGACGTTGGAGTCCACGTTCTTTAATAGTGGACTCTTGTTCCAAAC | |
| TGGAACAACACTCAACCCTATCTCGGGCTATTCTTTTGATTTATAAGGGATTTTGCCGATTTCGGAACCACCATCAA | |
| ACAGGATTTTCGCCTGCTGGGGCAAACCAGCGTGGACCGCTTGCTGCAACTCTCTCAGGGCCAGGCGGTGAAGGGCA | |
| ATCAGCTGTTGCCCGTCTCACTGGTGAAAAGAAAAACCACCCTGGATCCAAGCTTGCAGGTGGCACTTTTCGGGGAA | |
| ATGTGCGCGGAACCCCTATTTGTTTATTTTTCTAAATACATTCAAATATGTATCCGCTCATGAGACAATAACCCTGA | |
| TAAATGCTTCAATAATATTGAAAAAGGAAGAGTATGAGTATTCAACATTTCCGTGTCGCCCTTATTCCCTTTTTTGC | |
| GGCATTTTGCCTTCCTGTTTTTGCTCACCCAGAAACGCTGGTGAAAGTAAAAGATGCTGAAGATCAGTTGGGCGCAC | |
| GAGTGGGTTACATCGAACTGGATCTCAACAGCGGTAAGATCCTTGAGAGTTTTCGCCCCGAAGAACGTTTTCCAATG | |
| ATGAGCACTTTTAAAGTTCTGCTATGTCATACACTATTATCCCGTATTGACGCCGGGCAAGAGCAACTCGGTCGCCG | |
| GGCGCGGTATTCTCAGAATGACTTGGTTGAGTACTCACCAGTCACAGAAAAGCATCTTACGGATGGCATGACAGTAA | |
| GAGAATTATGCAGTGCTGCCATAACCATGAGTGATAACACTGCGGCCAACTTACTTCTGACAACGATCGGAGGACCG | |
| AAGGAGCTAACCGCTTTTTTGCACAACATGGGGGATCATGTAACTCGCCTTGATCGTTGGGAACCGGAGCTGAATGA | |
| AGCCATACCAAACGACGAGCGTGACACCACGATGCCTGTAGCAATGCCAACAACGTTGCGCAAACTATTAACTGGCG | |
| AACTACTTACTCTAGCTTCCCGGCAACAATTAATAGACTGGATGGAGGCGGATAAAGTTGCAGGACCACTTCTGCGC | |
| TCGGCCCTTCCGGCTGGCTGGTTTATTGCTGATAAATCTGGAGCCGGTGAGCGTGGGTCTCGCGGTATCATTGCAGC | |
| ACTGGGGCCAGATGGTAAGCCCTCCCGTATCGTAGTTATCTACACGACGGGGAGTCAGGCAACTATGGATGAACGAA | |
| ATAGACAGATCGCTGAGATAGGTGCCTCACTGATTAAGCATTGGTAACTGTCAGACCAAGTTTACTCATATATACTT | |
| TAGATTGATTTAAAACTTCATTTTTAATTTAAAAGGATCTAGGTGAAGATCCTTTTTGATAATCTCATGACCAAAAT | |
| CCCTTAACGTGAGTTTTCGTTCCACTGTACGTAAGACCCCCAAGCTTGTCGACTGAATGGCGAATGGCGCTTTGCCT | |
| GGTTTCCGGCACCAGAAGCGGTGCCGGAAAGCTGGCTGGAGTGCGATCTTCCTGAGGCCGATACTGTCGTCGTCCCC | |
| TCAAACTGGCAGATGCACGGTTACGATGCGCCCATCTACACCAACGTAACCTATCCCATTACGGTCAATCCGCCGTT | |
| TGTTCCCACGGAGAATCCGACGGGTTGTTACTCGCTCACATTTAATGTTGATGAAAGCTGGCTACAGGAAGGCCAGA | |
| CGCGAATTATTTTTGATGGCGTTCCTATTGGTTAAAAAATGAGCTGATTTAACAAAAATTTAACGCGAATTTTAACA | |
| AAATATTAACGTTTACAATTTAAATATTTGCTTATACAATCTTCCTGTTTTTGGGGCTTTTCTGATTATCAACCGGG | |
| GTACATATGATTGACATGCTAGTTTTACGATTACCGTTCATCGATTCTCTTGTTTGCTCCAGACTCTCAGGCAATGA | |
| CCTGATAGCCTTTGTAGATCTCTCAAAAATAGCTACCCTCTCCGGCATGAATTTATCAGCTAGAACGGTTGAATATC | |
| ATATTGATGGTGATTTGACTGTCTCCGGCCTTTCTCACCCTTTTGAATCTTTACCTACACATTACTCAGGCATTGCA | |
| TTTAAAATATATGAGGGTTCTAAAAATTTTTATCCTTGCGTTGAAATAAAGGCTTCTCCCGCAAAAGTATTACAGGG | |
| TCATAATGTTTTTGGTACAACCGATTTAGCTTTATGCTCTGAGGCTTTATTGCTTAATTTTGCTAATTCTTTGCCTT | |
| GCCTGTATGATTTATTGGATGTT | |

TABLE 5
pRH05 nucleotide sequence.

| | |
|---|---|
| AATGCTACTACTATTAGTAGAATTGATGCCACCTTTTCAGCTCGCGCCCCAAATGAAAATATAGCTAAACAGGTTAT | (SEQ ID NO:7) |
| TGACCATTTGCGAAATGTATCTAATGGTCAAACTAAATCTACTCGTTCGCAGAATTGGGAATCAACTGTTACATGGA | |
| ATGAAACTTCCAGACACCGTACTTTAGTTGCATATTTAAAACATGTTGAGCTACAGCACCAGATTCAGCAATTAAGC | |
| TCTAAGCCATCCGCAAAAATGACCTCTTATCAAAAGGAGCAATTAAAGGTACTCTCTAATCCTGACCTGTTGGAGTT | |
| TGCTTCCGGTCTGGTTCGCTTTGAAGCTCGAATTAAAACGCGATATTTGAAGTCTTTCGGGCTTCCTCTTAATCTTT | |
| TTGATGCAATCCGCTTTGCTTCTGACTATAATAGTCAGGGTAAAGACCTGATTTTTGATTTATGGTCATTCTCGTTT | |
| TCTGAACTGTTTAAAGCATTTGAGGGGGATTCAATGAATATTTATGACGATTCCGCAGTATTGGACGCTATCCAGTC | |
| TAAACATTTTACTATTACCCCCTCTGGCAAAACTTCTTTTGCAAAAGCCTCTCGCTATTTTGGTTTTTATCGTCGTC | |
| TGGTAAACGAGGGTTATGATAGTGTTGCTCTTACTATGCCTCGTAATTCCTTTTGGCGTTATGTATCTGCATTAGTT | |
| GAATGTGGTATTCCTAAATCTCAACTGATGAATCTTTCTACCTGTAATAATGTTGTTCCGTTAGTTCGTTTTATTAA | |
| CGTAGATTTTTCTTCCCAACGTCCTGACTGGTATAATGAGCCAGTTCTTAAAATCGCATAAGGTAATTCACAATGAT | |
| TAAAGTTGAAATTAAACCATCTCAAGCCCAATTTACTACTCGTTCTGGTGTTTCTCGTCAGGGCAAGCCTTATTCAC | |
| TGAATGAGCAGCTTTGTTACGTTGATTTGGGTAATGAATATCCGGTTCTTGTCAAGATTACTCTTGATGAAGGTCAG | |
| CCAGCCTATGCGCCTGGTCTGTACACCGTTCATCTGTCCTCTTTCAAAGTTGGTCAGTTCGGTTCCCTTATGATTGA | |
| CCGTCTGCGCCTCGTTCCGGCTAAGTAACATGGAGCAGGTCGCGGATTTCGACACAATTTATCAGGCGATGATACAA | |
| ATCTCCGTTGTACTTTGTTTCGCGCTTGGTATAATCGCTGGGGGTCAAAGATGAGTGTTTTAGTGTATTCTTTCGCC | |
| TCTTTCGTTTTAGGTTGGTGCCTTCGTAGTGGCATTACGTATTTTACCCGTTTAATGGAAACTTCCTCATGAAAAAG | |
| TCTTTAGTCCTCAAAGCCTCTGTAGCCGTTGCTACCCTCGTTCCGATGCTGTCTTTCGCTGCTGAGGGTGACGATCC | |
| CGCAAAAGCGGCCTTTAACTCCCTGCAAGCCTCAGCGACCGAATATATCGGTTATGCGTGGGCGATGGTTGTTGTCA | |
| TTGTCGGCGCAACTATCGGTATCAAGCTGTTTAAGAAATTCACCTCGAAAGCAAGCTGATAAACCGATACAATTAAA | |
| GGCTCCTTTTGGAGCCTTTTTTTTTGGAGATTTTCAACGTGAAAAAATTATTATTCGCAATTCCTTTAGTTGTTCCT | |
| TTCTATTCTCACAGTGCACAATCACATCTAGACGCGGCCGCTCATCACCACCATCATCACTCTGCTGAACAAAAACT | |
| CATCTCAGAAGAGGATCTGAATGGTGCCGCACAAGCGAGCTCTGCTTCCGGTGATTTTGATTATGAAAAGATGGCAA | |
| ACGCTAATAAGGGGGCTATGACCGAAAATGCCGATGAAAACGCGCTACAGTCTGACGCTAAAGGCAAACTTGATTCT | |
| GTCGCTACTGATTACGGTGCTGCTATCGATGGTTTCATTGGTGACGTTTCCGGCCTTGCTAATGGTAATGGTGCTAC | |
| TGGTGATTTTGCTGGCTCTAATTCCCAAATGGCTCAAGTCGGTGACGGTGATAATTCACCTTTAATGAATAATTTCC | |
| GTCAATATTTACCTTCCCTCCCTCAATCGGTTGAATGTCGCCCTTTTGTCTTTGGCGCTGGTAAACCATATGAATTT | |
| TCTATTGATTGTGACAAAATAAACTTATTCCGTGGTGTCTTTGCGTTTCTTTTATATGTTGCCACCTTTATGTATGT | |
| ATTTTCTACGTTTGCTAACATACTGCGTAATAAGGAGTCTTAATCATGCCAGTTCTTTTGGGTATTCCGTTATTATT | |
| GCGTTTCCTCGGTTTCCTTCTGGTAACTTTGTTCGGCTATCTGCTTACTTTTCTTAAAAAGGGCTTCGGTAAGATAG | |
| CTATTGCTATTTCATTGTTTCTTGCTCTTATTATTGGGCTTAACTCAATTCTTGTGGGTTATCTCTCTGATATTAGC | |
| GCTCAATTACCCTCTGACTTTGTTCAGGGTGTTCAGTTAATTCTCCCGTCTAATGCGCTTCCCTGTTTTTATGTTAT | |
| TCTCTCTGTAAAGGCTGCTATTTTCATTTTTGACGTTAAACAAAAAATCGTTTCTTATTTGGATTGGGATAAATAAT | |
| ATGGCTGTTTATTTTGTAACTGGCAAATTAGGCTCTGGAAAGACGCTCGTTAGCGTTGGTAAGATTCAGGATAAAAT | |
| TGTAGCTGGGTGCAAAATAGCAACTAATCTTGATTTAAGGCTTCAAAACCTCCCGCAAGTCGGGAGGTTCGCTAAAA | |
| CGCCTCGCGTTCTTAGAATACCGGATAAGCCTTCTATATCTGATTTGCTTGCTATTGGGCGCGGTAATGATTCCTAC | |
| GATGAAAATAAAAACGGCTTGCTTGTTCTCGATGAGTGCGGTACTTGGTTTAATACCCGTTCTTGGAATGATAAGGA | |
| AAGACAGCCGATTATTGATTGGTTTCTACATGCTCGTAAATTAGGATGGGATATTATTTTTCTTGTTCAGGACTTAT | |
| CTATTGTTGATAAACAGGCGCGTTCTGCATTAGCTGAACATGTTGTTTATTGTCGTCGTCTGGACAGAATTACTTTA | |
| CCTTTTGTCGGTACTTTATATTCTCTTATTACTGGCTCGAAAATGCCTCTGCCTAAATTACATGTTGGCGTTGTTAA | |
| ATATGGCGATTCTCAATTAAGCCCTACTGTTGAGCGTTGGCTTTATACTGGTAAGAATTTGTATAACGCATATGATA | |
| CTAAACAGGCTTTTTCTAGTAATTATGATTCCGGTGTTTATTCTTATTTAACGCCTTATTTATCACACGGTCGGTAT | |
| TTCAAACCATTAAATTTAGGTCAGAAGATGAAATTAACTAAAATATATTTGAAAAAGTTTTCTCGCGTTCTTTGTCT | |
| TGCGATTGGATTTGCATCAGCATTTACATATAGTTATATAACCCAACCTAAGCCGGAGGTTAAAAAGGTAGTCTCTC | |
| AGACCTATGATTTTGATAAATTCACTATTGACTCTTCTCAGCGTCTTAATCTAAGCTATCGCTATGTTTTCAAGGAT | |
| TCTAAGGGAAAATTAATTAATAGCGACGATTTACAGAAGCAAGGTTATTCACTCACATATATTGATTTATGTACTGT | |
| TTCCATTAAAAAAGGTAATTCAAATGAAATTGTTAAATGTAATTAATTTTGTTTTCTTGATGTTTGTTTCATCATCT | |
| TCTTTTGCTCAGGTAATTGAAATGAATAATTCGCCTCTGCGCGATTTTGTAACTTGGTATTCAAAGCAATCAGGCGA | |
| ATCCGTTATTGTTTCTCCCGATGTAAAAGGTACTGTTACTGTATATTCATCTGACGTTAAACCTGAAAATCTACGCA | |
| ATTTCTTTATTTCTGTTTTACGTGCAAATAATTTTGATATGGTAGGTTCTAACCCTTCCATTATTCAGAAGTATAAT | |
| CCAAACAATCAGGATTATATTGATGAATTGCCATCATCTGATAATCAGGAATATGATGATAATTCCGCTCCTTCTGG | |
| TGGTTTCTTTGTTCCGCAAAATGATAATGTTACTCAAACTTTTAAAATTAATAACGTTCGGGCAAAGGATTTAATAC | |
| GAGTTGTCGAATTGTTTGTAAAGTCTAATACTTCTAAATCCTCAAATGTATTATCTATTGACGGCTCTAATCTATTA | |
| GTTGTTAGTGCTCCTAAAGATATTTTAGATAACCTTCCTCAATTCCTTTCAACTGTTGATTTGCCAACTGACCAGAT | |
| ATTGATTGAGGGTTTGATATTTGAGGTTCAGCAAGGTGATGCTTTAGATTTTTCATTTGCTGCTGGCTCTCAGCGTG | |
| GCACTGTTGCAGGCGGTGTTAATACTGACCGCCTCACCTCTGTTTTATCTTCTGCTGGTGGTTCGTTCGGTATTTTT | |
| AATGGCGATGTTTTAGGGCTATCAGTTCGCGCATTAAAGACTAATAGCCATTCAAAAATATTGTCTGTGCCACGTAT | |
| TCTTACGCTTTCAGGTCAGAAGGGTTCTATCTCTGTTGGCCAGAATGTCCCTTTTATTACTGGTCGTGTGACTGGTG | |
| AATCTGCCAATGTAAATAATCCATTTCAGACGATTGAGCGTCAAAATGTAGGTATTTCCATGAGCGTTTTTCCTGTT | |
| GCAATGGCTGGCGGTAATATTGTTCTGGATATTACCAGCAAGGCCGATAGTTTGAGTTCTTCTACTCAGGCAAGTGA | |
| TGTTATTACTAATCAAAGAAGTATTGCTACAACGGTTAATTTGCGTGATGGACAGACTCTTTTACTCGGTGGCCTCA | |
| CTGATTATAAAAACACTTCTCAGGATTCTGGCGTACCGTTCCTGTCTAAAATCCCTTTAATCGGCCTCCTGTTTAGC | |
| TCCCGCTCTGATTCTAACGAGGAAAGCACGTTATACGTGCTCGTCAAAGCAACCATAGTACGCGCCCTGTAGCGGCG | |
| CATTAAGCGCGGCGGGTGTGGTGGTTACGCGCAGCGTGACCGCTACACTTGCCAGCGCCCTAGCGCCCGCTCCTTTC | |
| GCTTTCTTCCCTTCCTTTCTCGCCACGTTCGCCGGCTTTCCCCGTCAAGCTCTAAATCGGGGGCTCCCTTTAGGGTT | |
| CCGATTTAGTGCTTTACGGCACCTCGACCCCAAAAAACTTGATTTGGGTGATGGTTCACGTAGTGGGCCATCGCCCT | |
| GATAGACGGTTTTTCGCCCTTTGACGTTGGAGTCCACGTTCTTTAATAGTGGACTCTTGTTCCAAACTGGAACAACA | |
| CTCAACCCTATCTCGGGCTATTCTTTTGATTTATAAGGGATTTTGCCGATTTCGGAACCACCATCAAACAGGATTTT | |
| CGCCTGCTGGGGCAAACCAGCGTGGACCGCTTGCTGCAACTCTCTCAGGGCCAGGCGGTGAAGGGCAATCAGCTGTT | |
| GCCCGTCTCACTGGTGAAAAGAAAAACCACCCTGGATCCAAGCTTGCAGGTGGCACTTTTCGGGGAAATGTGCGCGG | |
| AACCCCTATTTGTTTATTTTTCTAAATACATTCAAATATGTATCCGCTCATGAGACAATAACCCTGATAAATGCTTC | |
| AATAATATTGAAAAAGGAAGAGTATGAGTATTCAACATTTCCGTGTCGCCCTTATTCCCTTTTTTGCGGCATTTTGC | |
| CTTCCTGTTTTTGCTCACCCAGAAACGCTGGTGAAAGTAAAAGATGCTGAAGATCAGTTGGGCGCACTAGTGGGTTA | |
| CATCGAACTGGATCTCAACAGCGGTAAGATCCTTGAGAGTTTTCGCCCCGAAGAACGTTTTCCAATGATGAGCACTT | |
| TTAAAGTTCTGCTATGTGGCGCGGTATTATCCCGTATTGACGCCGGGCAAGAGCAACTCGGTCGCCGCATACACTAT | |
| TCTCAGAATGACTTGGTTGAGTACTCACCAGTCACAGAAAAGCATCTTACGGATGGCATGACAGTAAGAGAATTATG | |
| CAGTGCTGCCATAACCATGAGTGATAACACTGCGGCCAACTTACTTCTGACAACGATCGGAGGACCGAAGGAGCTAA | |
| CCGCTTTTTTGCACAACATGGGGGATCATGTAACTCGCCTTGATCGTTGGGAACCGGAGCTGAATGAAGCCATACCA | |
| AACGACGAGCGTGACACCACGATGCCTGTAGCAATGGCAACAACGTTGCGCAAACTATTAACTGGCGAACTACTTAC | |
| TCTAGCTTCCCGGCAACAATTAATAGACTGGATGGAGGCGGATAAAGTTGCAGGACCACTTCTGCGCTCGGCCCTTC | |
| CGGCTGGCTGGTTTATTGCTGATAAATCTGGAGCCGGTGAGCGTGGGTCTCGCGGTATCATTGCAGCACTGGGGCCA | |
| GATGGTAAGCCCTCCCGTATCGTAGTTATCTACACGACGGGGAGTCAGGCAACTATGGATGAACGAAATAGACAGAT | |
| CGCTGAGATAGGTGCCTCACTGATTAAGCATTGGTAACTGTCAGACCAAGTTTACTCATATATACTTTAGATTGATT | |
| TAAAACTTCATTTTTAATTTAAAAGGATCTAGGTGAAGATCCTTTTTGATAATCTCATGACCAAAATCCCTTAACGT | |
| GAGTTTTCGTTCCACTGTACGTAAGACCCCCAAGCTTGTCGACCGCAACGCAATTAATGTGAGTTAGCTCACTCATT | |
| AGGCACCCCAGGCTTTACACTTTATGCTTCCGGCTCGTATGTTGTGTGGAATTGTGAGCGGATAACAATTTCACCCA | |
| TGCTTTGGACAGGAAACAGCTATGAAAAAGCTTTTATTCGCTATCCCGTTAGTTGTACCGTTCTATTCTCACTCTGC | |
| CGAGACAGTCGAATCCTGCCTGGCCAAGGTCCACACTGAGAATAGTTTCACAAATGTGTGGAAGGATGATAAGACCC | |
| TTGATCGATATGCCAATTACGAAGGCTGCTTATGGAATGCCACCGGCGTCGTTGTCTGCACGGGCGATGAGACACAA | |
| TGCTATGGCACGTGGGTGCCGATAGGCTTAGCCATACCGGAGAACGAAGGCGGCGGTAGCGAAGGCGGTGGCAGCGA | |
| AGGCGGTGGATCCGAAGGAGGTGGAACCAAGCCGCCGGAATATGGCGACACTCCGATACCTGGTTACACCTACATTA | |
| ATCCGTTAGATGGAACCTACCCTCCGGGCACCGAACAGAATCCTGCCAACCCGAACCCAAGCTTAGAAGAAAGCCAA | |
| CCGTTAAACACCTTTATGTTCCAAAACAACCGTTTTAGGAACCGTCAAGGTGCTCTTACCGTGTACACTGGAACCGT | |
| CACCCAGGGTACCGATCCTGTCAAGACCTACTATCAATATACCCCGGTCTCGAGTAAGGCTATGTACGATGCCTATT | |
| GGAATGGCAAGTTTCGTGATTGTGCCTTTCACAGCGGTTTCAACGAAGACCCTTTTGTCTGCGAGTACCAGGGTCAG | |
| AGTAGCGATTTACCGCAGCCACCGGTTAACGCGGGTGGTGGTAGCGGCGGAGGCAGCGGCGGTGGTAGCGAAGGCGG | |
| AGGTAGCGAAGGAGGTGGCAGCGGAGGCGGTAGCGGCAGTGGCGACTTCGACTACGAGAAAATGGCTAATGCCAACA | |
| AAGGCGCCATGACTGAGAACGCTGACGAGAATGCACTGCAAAGTGATGCCAAGGGTAAGTTAGACAGCGTCGCCACA | |
| GACTATGGTGCTGCCATCGACGGCTTTATCGGCGATGTCAGTGGTCTGGCTAACGGCAACGGAGCCACCGGAGACTT | |
| CGCAGGTTCGAATTCTCAGATGGCCCAGGTTGGAGATGGGGACAACAGTCCGCTTATGAACAACTTTAGACAGTACC | |
| TTCCGTCTCTTCCGCAGAGTGTCGAGTGCCGTCCATTCGTTTTCGGAGCCGGCAAGCCTTACGAGTTCAGCATCGAC | |
| TGCGATAAGATCAATCTTTTCCGCGGCGTTTTCGCTTTCTTGCTATACGTCGCTACTTTCATGTACGTTTTCAGCAC | |
| TTTCGCCAATATTTTACGCAACAAAGAAAGCTAGTGATCTCCTAGGAAGCCCGCCTAATGAGCGGGCTTTTTTTTTC | |
| TGGTATGCATCCTGAGGCCGATACTGTCGTCGTCCCCTCAAACTGGCAGATGCACGGTTACGATGCGCCCATCTACA | |
| CCAACGTGACCTATCCCATTACGGTCAATCCGCCGTTTGTTCCCACGGAGAATCCGACGGGTTGTTACTCGCTCACA | |
| TTTAATGTTGATGAAAGCTGGCTACAGGAAGGCCAGACGCGAATTATTTTTGATGGCGTTCCTATTGGTTAAAAAAT | |
| GAGCTGATTTAACAAAAATTTAATGCGAATTTTAACAAAATATTAACGTTTACAATTTAAATATTTGCTTATACAAT | |
| CTTCCTGTTTTTGGGGCTTTTCTGATTATCAACCGGGGTACATATGATTGACATGCTAGTTTTACGATTACCGTTCA | |
| TCGATTCTCTTGTTTGCTCCAGACTCTCAGGCAATGACCTGATAGCCTTTGTAGATCTCTCAAAAATAGCTACCCTC | |
| TCCGGCATGAATTTATCAGCTAGAACGGTTGAATATCATATTGATGGTGATTTGACTGTCTCCGGCCTTTCTCACCC | |
| TTTTGAATCTTTACCTACACATTACTCAGGCATTGCATTTAAAATATATGAGGGTTCTAAAAATTTTTATCCTTGCG | |
| TTGAAATAAAGGCTTCTCCCGCAAAAGTATTACAGGGTCATAATGTTTTTGGTACAACCGATTTAGCTTTATGCTCT | |
| GAGGCTTTATTGCTTAATTTTGCTAATTCTTTGCCTTGCCTGTATGATTTATTGGATGTT | |

TABLE 6
DY3F31 nucleotide sequence

| | | |
|---|---|---|
| 1 | AATGCTACTA CTATTAGTAG AATTGATGCC ACCTTTTCAG CTCGCGCCCC AAATGAAAAT | (SEQ ID NO:8) |
| 61 | ATAGCTAAAC AGGTTATTGA CCATTTGCGA AATGTATCTA ATGGTCAAAC TAAATCTACT | |
| 121 | CGTTCGCAGA ATTGGGAATC AACTGTTATA TGGAATGAAA CTTCCAGACA CCGTACTTTA | |
| 181 | GTTGCATATT TAAAACATGT TGAGCTACAG CATTATATTC AGCAATTAAG CTCTAAGCCA | |
| 241 | TCCGCAAAAA TGACCTCTTA TCAAAAGGAG CAATTAAAGG TACTCTCTAA TCCTGACCTG | |
| 301 | TTGGAGTTTG CTTCCGGTCT GGTTCGCTTT GAAGCTCGAA TTAAAACGCG ATATTTGAAG | |
| 361 | TCTTTCGGGC TTCCTCTTAA TCTTTTTGAT GCAATCCGCT TTGCTTCTGA CTATAATAGT | |
| 421 | CAGGGTAAAG ACCTGATTTT TGATTTATGG TCATTCTCGT TTTCTGAACT GTTTAAAGCA | |
| 481 | TTTGAGGGGG ATTCAATGAA TATTTATGAC GATTCCGCAG TATTGGACGC TATCCAGTCT | |
| 541 | AAACATTTTA CTATTACCCC CTCTGGCAAA ACTTCTTTTG CAAAAGCCTC TCGCTATTTT | |
| 601 | GGTTTTTATC GTCGTCTGGT AAACGAGGGT TATGATAGTG TTGCTCTTAC TATGCCTCGT | |
| 661 | AATTCCTTTT GGCGTTATGT ATCTGCATTA GTTGAATGTG GTATTCCTAA ATCTCAACTG | |
| 721 | ATGAATCTTT CTACCTGTAA TAATGTTGTT CCGTTAGTTC GTTTTATTAA CGTAGATTTT | |
| 781 | TCTTCCCAAC GTCCTGACTG GTATAATGAG CCAGTTCTTA AAATCGCATA AGGTAATTCA | |
| 841 | CAATGATTAA AGTTGAAATT AAACCATCTC AAGCCCAATT TACTACTCGT TCTGGTGTTT | |
| 901 | CTCGTCAGGG CAAGCCTTAT TCACTGAATG AGCAGCTTTG TTACGTTGAT TTGGGTAATG | |
| 961 | AATATCCGGT TCTTGTCAAG ATTACTCTTG ATGAAGGTCA GCCAGCCTAT GCGCCTGGTC | |
| 1021 | TGTACACCGT TCATCTGTCC TCTTTCAAAG TTGGTCAGTT CGGTTCCCTT ATGATTGACC | |
| 1081 | GTCTGCGCCT CGTTCCGGCT AAGTAACATG GAGCAGGTCG CGGATTTCGA CACAATTTAT | |
| 1141 | CAGGCGATGA TACAAATCTC CGTTGTACTT TGTTTCGCGC TTGGTATAAT CGCTGGGGGT | |
| 1201 | CAAAGATGAG TGTTTTAGTG TATTCTTTTG CCTCTTTCGT TTTAGGTTGG TGCCTTCGTA | |
| 1261 | GTGGCATTAC GTATTTTACC CGTTTAATGG AAACTTCCTC ATGAAAAAGT CTTTAGTCCT | |
| 1321 | CAAAGCCTCT GTAGCCGTTG CTACCCTCGT TCCGATGCTG TCTTTCGCTG CTGAGGGTGA | |
| 1381 | CGATCCCGCA AAAGCGGCCT TTAACTCCCT GCAAGCCTCA GCGACCGAAT ATATCGGTTA | |
| 1441 | TGCGTGGGCG ATGGTTGTTG TCATTGTCGG CGCAACTATC GGTATCAAGC TGTTTAAGAA | |
| 1501 | ATTCACCTCG AAAGCAAGCT GATAAACCGA TACAATTAAA GGCTCCTTTT GGAGCCTTTT | |
| 1561 | TTTTGGAGAT TTTCAACGTG AAAAAATTAT TATTCGCAAT TCCTTTAGTT GTTCCTTTCT | |
| 1621 | ATTCTGGCGC GGCCGAATCA CATCTAGACG GCGCCGCTGA AACTGTTGAA AGTTGTTTAG | |
| 1681 | CAAAATCCCA TACAGAAAAT TCATTTACTA ACGTCTGGAA AGACGACAAA ACTTTAGATC | |
| 1741 | GTTACGCTAA CTATGAGGGC TGTCTGTGGA ATGCTACAGG CGTTGTAGTT TGTACTGGTG | |
| 1801 | ACGAAACTCA GTGTTACGGT ACATGGGTTC CTATTGGGCT TGCTATCCCT GAAAATGAGG | |
| 1861 | GTGGTGGCTC TGAGGGTGGC GGTTCTGAGG GTGGCGGTTC TGAGGGTGGC GGTACTAAAC | |
| 1921 | CTCCTGAGTA CGGTGATACA CCTATTCCGG GCTATACTTA TATCAACCCT CTCGACGGCA | |
| 1981 | CTTATCCGCC TGGTACTGAG CAAAACCCCG CTAATCCTAA TCCTTCTCTT GAGGAGTCTC | |
| 2041 | AGCCTCTTAA TACTTTCATG TTTCAGAATA ATAGGTTCCG AAATAGGCAG GGGGCATTAA | |
| 2101 | CTGTTTATAC GGGCACTGTT ACTCAAGGCA CTGACCCCGT TAAAACTTAT TACCAGTACA | |
| 2161 | CTCCTGTATC ATCAAAAGCC ATGTATGACG CTTACTGGAA CGGTAAATTC AGAGACTGCG | |
| 2221 | CTTTCCATTC TGGCTTTAAT GAGGATTTAT TTGTTTGTGA ATATCAAGGC CAATCGTCTG | |
| 2281 | ACCTGCCTCA ACCTCCTGTC AATGCTGGCG GCGGCTCTGG TGGTGGTTCT GGTGGCGGCT | |
| 2341 | CTGAGGGTGG TGGCTCTGAG GGAGGCGGTT CCGGTGGTGG CTCTGGTTCC GGTGATTTTG | |
| 2401 | ATTATGAAAA GATGGCAAAC GCTAATAAGG GGGCTATGAC CGAAAATGCC GATGAAAACG | |
| 2461 | CGCTACAGTC TGACGCTAAA GGCAAACTTG ATTCTGTCGC TACTGATTAC GGTGCTGCTA | |
| 2521 | TCGATGGTTT CATTGGTGAC GTTTCCGGCC TTGCTAATGG TAATGGTGCT ACTGGTGATT | |
| 2581 | TTGCTGGCTC TAATTCCCAA ATGGCTCAAG TCGGTGACGG TGATAATTCA CCTTTAATGA | |
| 2641 | ATAATTTCCG TCAATATTTA CCTTCCCTCC CTCAATCGGT TGAATGTCGC CCTTTTGTCT | |
| 2701 | TTGGCGCTGG TAAACCATAT GAATTTTCTA TTGATTGTGA CAAAATAAAC TTATTCCGTG | |
| 2761 | GTGTCTTTGC GTTTCTTTTA TATGTTGCCA CCTTTATGTA TGTATTTTCT ACGTTTGCTA | |
| 2821 | ACATACTGCG TAATAAGGAG TCTTAATCAT GCCAGTTCTT TTGGGTATTC CGTTATTATT | |
| 2881 | GCGTTTCCTC GGTTTCCTTC TGGTAACTTT GTTCGGCTAT CTGCTTACTT TTCTTAAAAA | |
| 2941 | GGGCTTCGGT AAGATAGCTA TTGCTATTTC ATTGTTTCTT GCTCTTATTA TTGGGCTTAA | |
| 3001 | CTCAATTCTT GTGGGTTATC TCTCTGATAT TAGCGCTCAA TTACCCTCTG ACTTTGTTCA | |
| 3061 | GGGTGTTCAG TTAATTCTCC CGTCTAATGC GCTTCCCTGT TTTTATGTTA TTCTCTCTGT | |
| 3121 | AAAGGCTGCT ATTTTCATTT TTGACGTTAA ACAAAAAATC GTTTCTTATT TGGATTGGGA | |
| 3181 | TAAATAATAT GGCTGTTTAT TTTGTAACTG GCAAATTAGG CTCTGGAAAG ACGCTCGTTA | |
| 3241 | GCGTTGGTAA GATTCAGGAT AAAATTGTAG CTGGGTGCAA AATAGCAACT AATCTTGATT | |
| 3301 | TAAGGCTTCA AAACCTCCCG CAAGTCGGGA GGTTCGCTAA AACGCCTCGC GTTCTTAGAA | |
| 3361 | TACCGGATAA GCCTTCTATA TCTGATTTGC TTGCTATTGG GCGCGGTAAT GATTCCTACG | |
| 3421 | ATGAAAATAA AAACGGCTTG CTTGTTCTCG ATGAGTGCGG TACTTGGTTT AATACCCGTT | |
| 3481 | CTTGGAATGA TAAGGAAAGA CAGCCGATTA TTGATTGGTT TCTACATGCT CGTAAATTAG | |
| 3541 | GATGGGATAT TATTTTTCTT GTTCAGGACT TATCTATTGT TGATAAACAG GCGCGTTCTG | |
| 3601 | CATTAGCTGA ACATGTTGTT TATTGTCGTC GTCTGGACAG AATTACTTTA CCTTTTGTCG | |
| 3661 | GTACTTTATA TTCTCTTATT ACTGGCTCGA AAATGCCTCT GCCTAAATTA CATGTTGGCG | |
| 3721 | TTGTTAAATA TGGCGATTCT CAATTAAGCC CTACTGTTGA GCGTTGGCTT TATACTGGTA | |
| 3781 | AGAATTTGTA TAACGCATAT GATACTAAAC AGGCTTTTTC TAGTAATTAT GATTCCGGTG | |
| 3841 | TTTATTCTTA TTTAACGCCT TATTTATCAC ACGGTCGGTA TTTCAAACCA TTAAATTTAG | |
| 3901 | GTCAGAAGAT GAAATTAACT AAAATATATT TGAAAAAGTT TTCTCGCGTT CTTTGTCTTG | |
| 3961 | CGATTGGATT TGCATCAGCA TTTACATATA GTTATATAAC CCAACCTAAG CCGGAGGTTA | |
| 4021 | AAAAGGTAGT CTCTCAGACC TATGATTTTG ATAAATTCAC TATTGACTCT TCTCAGCGTC | |
| 4081 | TTAATCTAAG CTATCGCTAT GTTTTCAAGG ATTCTAAGGG AAAATTAATT AATAGCGACG | |
| 4141 | ATTTACAGAA GCAAGGTTAT TCACTCACAT ATATTGATTT ATGTACTGTT TCCATTAAAA | |
| 4201 | AAGGTAATTC AAATGAAATT GTTAAATGTA ATTAATTTTG TTTTCTTGAT GTTTGTTTCA | |
| 4261 | TCATCTTCTT TTGCTCAGGT AATTGAAATG AATAATTCGC CTCTGCGCGA TTTTGTAACT | |
| 4321 | TGGTATTCAA AGCAATCAGG CGAATCCGTT ATTGTTTCTC CCGATGTAAA AGGTACTGTT | |
| 4381 | ACTGTATATT CATCTGACGT TAAACCTGAA AATCTACGCA ATTTCTTTAT TTCTGTTTTA | |
| 4441 | CGTGCAAATA ATTTTGATAT GGTAGGTTCT AACCCTTCCA TTATTCAGAA GTATAATCCA | |
| 4501 | AACAATCAGG ATTATATTGA TGAATTGCCA TCATCTGATA ATCAGGAATA TGATGATAAT | |
| 4561 | TCCGCTCCTT CTGGTGGTTT CTTTGTTCCG CAAAATGATA ATGTTACTCA AACTTTTAAA | |
| 4621 | ATTAATAACG TTCGGGCAAA GGATTTAATA CGAGTTGTCG AATTGTTTGT AAAGTCTAAT | |
| 4681 | ACTTCTAAAT CCTCAAATGT ATTATCTATT GACGGCTCTA ATCTATTAGT TGTTAGTGCT | |
| 4741 | CCTAAAGATA TTTTAGATAA CCTTCCTCAA TTCCTTTCAA CTGTTGATTT GCCAACTGAC | |
| 4801 | CAGATATTGA TTGAGGGTTT GATATTTGAG GTTCAGCAAG GTGATGCTTT AGATTTTTCA | |
| 4861 | TTTGCTGCTG GCTCTCAGCG TGGCACTGTT GCAGGCGGTG TTAATACTGA CCGCCTCACC | |
| 4921 | TCTGTTTTAT CTTCTGCTGG TGGTTCGTTC GGTATTTTTA ATGGCGATGT TTTAGGGCTA | |
| 4981 | TCAGTTCGCG CATTAAAGAC TAATAGCCAT TCAAAAATAT TGTCTGTGCC ACGTATTCTT | |
| 5041 | ACGCTTTCAG GTCAGAAGGG TTCTATCTCT GTTGGCCAGA ATGTCCCTTT TATTACTGGT | |
| 5101 | CGTGTGACTG GTGAATCTGC CAATGTAAAT AATCCATTTC AGACGATTGA GCGTCAAAAT | |
| 5161 | GTAGGTATTT CCATGAGCGT TTTTCCTGTT GCAATGGCTG GCGGTAATAT TGTTCTGGAT | |
| 5221 | ATTACCAGCA AGGCCGATAG TTTGAGTTCT TCTACTCAGG CAAGTGATGT TATTACTAAT | |
| 5281 | CAAAGAAGTA TTGCTACAAC GGTTAATTTG CGTGATGGAC AGACTCTTTT ACTCGGTGGC | |
| 5341 | CTCACTGATT ATAAAAACAC TTCTCAGGAT TCTGGCGTAC CGTTCCTGTC TAAAATCCCT | |
| 5401 | TTAATCGGCC TCCTGTTTAG CTCCCGCTCT GATTCTAACG AGGAAAGCAC GTTATACGTG | |
| 5461 | CTCGTCAAAG CAACCATAGT ACGCGCCCTG TAGCGGCGCA TTAAGCGCGG CGGGTGTGGT | |
| 5521 | GGTTACGCGC AGCGTGACCG CTACACTTGC CAGCGCCCTA GCGCCCGCTC CTTTCGCTTT | |
| 5581 | CTTCCCTTCC TTTCTCGCCA CGTTCGCCGG CTTTCCCCGT CAAGCTCTAA ATCGGGGGCT | |
| 5641 | CCCTTTAGGG TTCCGATTTA GTGCTTTACG GCACCTCGAC CCCAAAAAAC TTGATTTGGG | |
| 5701 | TGATGGTTCA CGTAGTGGGC CATCGCCCTG ATAGACGGTT TTTCGCCCTT TGACGTTGGA | |
| 5761 | GTCCACGTTC TTTAATAGTG GACTCTTGTT CCAAACTGGA ACAACACTCA ACCCTATCTC | |
| 5821 | GGGCTATTCT TTTGATTTAT AAGGGATTTT GCCGATTTCG GAACCACCAT CAAACAGGAT | |
| 5881 | TTTCGCCTGC TGGGGCAAAC CAGCGTGGAC CGCTTGCTGC AACTCTCTCA GGGCCAGGCG | |
| 5941 | GTGAAGGGCA ATCAGCTGTT GCCCGTCTCA CTGGTGAAAA GAAAAACCAC CCTGGATCCA | |
| 6001 | AGCTTGCAGG TGGCACTTTT CGGGGAAATG TGCGCGGAAC CCCTATTTGT TTATTTTTCT | |
| 6061 | AAATACATTC AAATATGTAT CCGCTCATGA GACAATAACC CTGATAAATG CTTCAATAAT | |
| 6121 | ATTGAAAAAG GAAGAGTATG AGTATTCAAC ATTTCCGTGT CGCCCTTATT CCCTTTTTTG | |
| 6181 | CGGCATTTTG CCTTCCTGTT TTTGCTCACC CAGAAACGCT GGTGAAAGTA AAAGATGCTG | |
| 6241 | AAGATCAGTT GGGCGCACTA GTGGGTTACA TCGAACTGGA TCTCAACAGC GGTAAGATCC | |
| 6301 | TTGAGAGTTT TCGCCCCGAA GAACGTTTTC CAATGATGAG CACTTTTAAA GTTCTGCTAT | |
| 6361 | GTGGCGCGGT ATTATCCCGT ATTGACGCCG GGCAAGAGCA ACTCGGTCGC CGCATACACT | |
| 6421 | ATTCTCAGAA TGACTTGGTT GAGTACTCAC CAGTCACAGA AAAGCATCTT ACGGATGGCA | |
| 6481 | TGACAGTAAG AGAATTATGC AGTGCTGCCA TAACCATGAG TGATAACACT GCGGCCAACT | |
| 6541 | TACTTCTGAC AACGATCGGA GGACCGAAGG AGCTAACCGC TTTTTTGCAC AACATGGGGG | |
| 6601 | ATCATGTAAC TCGCCTTGAT CGTTGGGAAC CGGAGCTGAA TGAAGCCATA CCAAACGACG | |
| 6661 | AGCGTGACAC CACGATGCCT GTAGCAATGG CAACAACGTT GCGCAAACTA TTAACTGGCG | |
| 6721 | AACTACTTAC TCTAGCTTCC CGGCAACAAT TAATAGACTG GATGGAGGCG GATAAAGTTG | |
| 6781 | CAGGACCACT TCTGCGCTCG GCCCTTCCGG CTGGCTGGTT TATTGCTGAT AAATCTGGAG | |
| 6841 | CCGGTGAGCG TGGGTCTCGC GGTATCATTG CAGCACTGGG GCCAGATGGT AAGCCCTCCC | |
| 6901 | GTATCGTAGT TATCTACACG ACGGGGAGTC AGGCAACTAT GGATGAACGA AATAGACAGA | |
| 6961 | TCGCTGAGAT AGGTGCCTCA CTGATTAAGC ATTGGTAACT GTCAGACCAA GTTTACTCAT | |
| 7021 | ATATACTTTA GATTGATTTA AAACTTCATT TTTAATTTAA AAGGATCTAG GTGAAGATCC | |
| 7081 | TTTTTGATAA TCTCATGACC AAAATCCCTT AACGTGAGTT TTCGTTCCAC TGTACGTAAG | |
| 7141 | ACCCCCAAGC TTGTCGACTG AATGGCGAAT GGCGCTTTGC CTGGTTTCCG GCACCAGAAG | |
| 7201 | CGGTGCCGGA AAGCTGGCTG GAGTGCGATC TTCCTGACGC TCGAGCGCAA CGCAATTAAT | |
| 7261 | GTGAGTTAGC TCACTCATTA GGCACCCCAG GCTTTACACT TTATGCTTCC GGCTCGTATG | |
| 7321 | TTGTGTGGAA TTGTGAGCGG ATAACAATTT CACACAGGAA ACAGCTATGA CCATGATTAC | |
| 7381 | GCCAAGCTTT GGAGCCTTTT TTTTGGAGAT TTTCAACGTG AAAAAATTAT TATTCGCAAT | |
| 7441 | TCCTTTAGTT GTTCCTTTCT ATTCTCACAG TGCACAGTGA TAGACTAGTT AGACGCGTGC | |
| 7501 | TTAAAGGCCT CCAATCCTCT TGGCGCGCCA ATTCTATTTC AAGGAGACAG TCATAATGAA | |
| 7561 | ATACCTATTG CCTACGGCAG CCGCTGGATT GTTATTACTC GCGGCCCAGC CGGCCCTCTG | |
| 7621 | ATAAGATATC ACTTGTTTAA ACTCTGCTTG GCCCTCTTGG CCTTCTAGTA GACTTGCGGC | |
| 7681 | CGCACATCAT CATCACCATC ACGGGGCCGC AGAACAAAAA CTCATCTCAG AAGAGGATCT | |
| 7741 | GAATGGGGCC GCATAGGCTA GCTCTGCTAG TGGCGACTTC GACTACGAGA AAATGGCTAA | |
| 7801 | TGCCAACAAA GGCGCCATGA CTGAGAACGC TGACGAGAAT GCTTTGCAAA GCGATGCCAA | |
| 7861 | GGGTAAGTTA GACAGCGTCG CGACCGACTA TGGCGCCGCC ATCGACGGCT TTATCGGCGA | |
| 7921 | TGTCAGTGGT TTGGCCAACG GCAACGGAGC CACCGGAGAC TTCGCAGGTT CGAATTCTCA | |
| 7981 | GATGGCCCAG GTTGGAGATG GGGACAACAG TCCGCTTATG AACAACTTTA GACAGTACCT | |
| 8041 | TCCGTCTCTT CCGCAGAGTG TCGAGTGCCG TCCATTCGTT TTCTCTGCCG GCAAGCCTTA | |
| 8101 | CGAGTTCAGC ATCGACTGCG ATAAGATCAA TCTTTTCCGC GGCGTTTTCG CTTTCTTGCT | |
| 8161 | ATACGTCGCT ACTTTCATGT ACGTTTTCAG CACTTTCGCC AATATTTTAC GCAACAAAGA | |
| 8221 | AAGCTAGTGA TCTCCTAGGA AGCCCGCCTA ATGAGCGGGC TTTTTTTTTC TGGTATGCAT | |
| 8281 | CCTGAGGCCG ATACTGTCGT CGTCCCCTCA AACTGGCAGA TGCACGGTTA CGATGCGCCC | |
| 8341 | ATCTACACCA ACGTGACCTA TCCCATTACG GTCAATCCGC CGTTTGTTCC CACGGAGAAT | |
| 8401 | CCGACGGGTT GTTACTCGCT CACATTTAAT GTTGATGAAA GCTGGCTACA GGAAGGCCAG | |
| 8461 | ACGCGAATTA TTTTTGATGG CGTTCCTATT GGTTAAAAAA TGAGCTGATT TAACAAAAAT | |
| 8521 | TTAATGCGAA TTTTAACAAA ATATTAACGT TTACAATTTA AATATTTGCT TATACAATCT | |
| 8581 | TCCTGTTTTT GGGGCTTTTC TGATTATCAA CCGGGGTACA TATGATTGAC ATGCTAGTTT | |
| 8641 | TACGATTACC GTTCATCGAT TCTCTTGTTT GCTCCAGACT CTCAGGCAAT GACCTGATAG | |
| 8701 | CCTTTGTAGA TCTCTCAAAA ATAGCTACCC TCTCCGGCAT TAATTTATCA GCTAGAACGG | |
| 8761 | TTGAATATCA TATTGATGGT GATTTGACTG TCTCCGGCCT TTCTCACCCT TTTGAATCTT | |
| 8821 | TACCTACACA TTACTCAGGC ATTGCATTTA AAATATATGA GGGTTCTAAA AATTTTTATC | |
| 8881 | CTTGCGTTGA AATAAAGGCT TCTCCCGCAA AAGTATTACA GGGTCATAAT GTTTTTGGTA | |
| 8941 | CAACCGATTT AGCTTTATGC TCTGAGGCTT TATTGCTTAA TTTTGCTAAT TCTTTGCCTT | |
| 9001 | GCCTGTATGA TTTATTGGAT GTT | |

TABLE 7
pCES1 nucleotide sequence.

| | | |
|---|---|---|
| 1 | GACGAAAGGG CCTCGTGATA CGCCTATTTT TATAGGTTAA TGTCATGATA ATAATGGTTT | (SEQ ID NO:9) |
| 61 | CTTAGACGTC AGGTGGCACT TTTCGGGGAA ATGTGCGCGG AACCCCTATT TGTTTATTTT | |
| 121 | TCTAAATACA TTCAAATATG TATCCGCTCA TGAGACAATA ACCCTGATAA ATGCTTCAAT | |
| 181 | AATATTGAAA AAGGAAGAGT ATGAGTATTC AACATTTCCG TGTCGCCCTT ATTCCCTTTT | |
| 241 | TTGCGGCATT TTGCCTTCCT GTTTTTGCTC ACCCAGAAAC GCTGGTGAAA GTAAAAGATG | |
| 301 | CTGAAGATCA GTTGGGTGCC CGAGTGGGTT ACATCGAACT GGATCTCAAC AGCGGTAAGA | |
| 361 | TCCTTGAGAG TTTTCGCCCC GAAGAACGTT TTCCAATGAT GAGCACTTTT AAAGTTCTGC | |
| 421 | TATGTGGCGC GGTATTATCC CGTATTGACG CCGGGCAAGA GCAACTCGGT CGCCGCATAC | |
| 481 | ACTATTCTCA GAATGACTTG GTTGAGTACT CACCAGTCAC AGAAAAGCAT CTTACGGATG | |
| 541 | GCATGACAGT AAGAGAATTA TGCAGTGCTG CCATAACCAT GAGTGATAAC ACTGCGGCCA | |
| 601 | ACTTACTTCT GACAACGATC GGAGGACCGA AGGAGCTAAC CGCTTTTTTG CACAACATGG | |
| 661 | GGGATCATGT AACTCGCCTT GATCGTTGGG AACCGGAGCT GAATGAAGCC ATACCAAACG | |
| 721 | ACGAGCGTGA CACCACGATG CCTGTAGCAA TGGCAACAAC GTTGCGCAAA CTATTAACTG | |
| 781 | GCGAACTACT TACTCTAGCT TCCCGGCAAC AATTAATAGA CTGGATGGAG GCGGATAAAG | |
| 841 | TTGCAGGACC ACTTCTGCGC TCGGCCCTTC CGGCTGGCTG GTTTATTGCT GATAAATCTG | |
| 901 | GAGCCGGTGA GCGTGGGTCT CGCGGTATCA TTGCAGCACT GGGGCCAGAT GGTAAGCCCT | |
| 961 | CCCGTATCGT AGTTATCTAC ACGACGGGGA GTCAGGCAAC TATGGATGAA CGAAATAGAC | |
| 1021 | AGATCGCTGA GATAGGTGCC TCACTGATTA AGCATTGGTA ACTGTCAGAC CAAGTTTACT | |
| 1081 | CATATATACT TTAGATTGAT TTAAAACTTC ATTTTTAATT TAAAAGGATC TAGGTGAAGA | |
| 1141 | TCCTTTTTGA TAATCTCATG ACCAAAATCC CTTAACGTGA GTTTTCGTTC CACTGAGCGT | |
| 1201 | CAGACCCCGT AGAAAAGATC AAAGGATCTT CTTGAGATCC TTTTTTTCTG CGCGTAATCT | |
| 1261 | GCTGCTTGCA AACAAAAAAA CCACCGCTAC CAGCGGTGGT TTGTTTGCCG GATCAAGAGC | |
| 1321 | TACCAACTCT TTTTCCGAAG GTAACTGGCT TCAGCAGAGC GCAGATACCA AATACTGTCC | |
| 1381 | TTCTAGTGTA GCCGTAGTTA GGCCACCACT TCAAGAACTC TGTAGCACCG CCTACATACC | |
| 1441 | TCGCTCTGCT AATCCTGTTA CCAGTGGCTG CTGCCAGTGG CGATAAGTCG TGTCTTACCG | |
| 1501 | GGTTGGACTC AAGACGATAG TTACCGGATA AGGCGCAGCG GTCGGGCTGA ACGGGGGGTT | |
| 1561 | CGTGCATACA GCCCAGCTTG GAGCGAACGA CCTACACCGA ACTGAGATAC CTACAGCGTG | |
| 1621 | AGCATTGAGA AAGCGCCACG CTTCCCGAAG GGAGAAAGGC GGACAGGTAT CCGGTAAGCG | |
| 1681 | GCAGGGTCGG AACAGGAGAG CGCACGAGGG AGCTTCCAGG GGGAAACGCC TGGTATCTTT | |
| 1741 | ATAGTCCTGT CGGGTTTCGC CACCTCTGAC TTGAGCGTCG ATTTTTGTGA TGCTCGTCAG | |
| 1801 | GGGGGCGGAG CCTATGGAAA AACGCCAGCA ACGCGGCCTT TTTACGGTTC CTGGCCTTTT | |
| 1861 | GCTGGCCTTT TGCTCACATG TTCTTTCCTG CGTTATCCCC TGATTCTGTG GATAACCGTA | |
| 1921 | TTACCGCCTT TGAGTGAGCT GATACCGCTC GCCGCAGCCG AACGACCGAG CGCAGCGAGT | |
| 1981 | CAGTGAGCGA GGAAGCGGAA GAGCGCCCAA TACGCAAACC GCCTCTCCCC GCGCGTTGGC | |
| 2041 | CGATTCATTA ATGCAGCTGG CACGACAGGT TTCCCGACTG GAAAGCGGGC AGTGAGCGCA | |
| 2101 | ACGCAATTAA TGTGAGTTAG CTCACTCATT AGGCACCCCA GGCTTTACAC TTTATGCTTC | |
| 2161 | CGGCTCGTAT GTTGTGTGGA ATTGTGAGCG GATAACAATT TCACACAGGA AACAGCTATG | |
| 2221 | ACCATGATTA CGCCAAGCTT TGGAGCCTTT TTTTTGGAGA TTTTCAACGT GAAAAAATTA | |
| 2281 | TTATTCGCAA TTCCTTTAGT TGTTCCTTTC TATTCTCACA GTGCACAGGT CCAACTGCAG | |
| 2341 | GTCGACCTCG AGATCAAACG TGGAACTGTG GCTGCACCAT CTGTCTTCAT CTTCCCGCCA | |
| 2401 | TCTGATGAGC AGTTGAAATC TGGAACTGCC TCTGTTGTGT GCCTGCTGAA TAACTTCTAT | |
| 2461 | CCCAGAGAGG CCAAAGTACA GTGGAAGGTG GATAACGCCC TCCAATCGGG TAACTCCCAG | |
| 2521 | GAGAGTGTCA CAGAGCAGGA CAGCAAGGAC AGCACCTACA GCCTCAGCAG CACCCTGACG | |
| 2581 | CTGAGCAAAG CAGACTACGA GAAACACAAA GTCTACGCCT GCGAAGTCAC CCATCAGGGC | |
| 2641 | CTGAGTTCAC CGGTGACAAA GAGCTTCAAC AGGGGAGAGT GTTAATAAGG CGCGCCAATT | |
| 2701 | CTATTTCAAG GAGACAGTCA TAATGAAATA CCTATTGCCT ACGGCAGCCG CTGGATTGTT | |
| 2761 | ATTACTCGCG GCCCAGCCGG CCATGGCCCA GGTGCAGCTG CAGGAGAGCG GGGTCACCGT | |
| 2821 | CTCAAGCGCC TCCACCAAGG GCCCATCGGT CTTCCCCCTG GCACCCTCCT CCAAGAGCAC | |
| 2881 | CTCTGGGGGC ACAGCGGCCC TGGGCTGCCT GGTCAAGGAC TACTTCCCCG AACCGGTGAC | |
| 2941 | GGTGTCGTGG AACTCAGGCG CCCTGACCAG CGGCGTCCAC ACCTTCCCGG CTGTCCTACA | |
| 3001 | GTCCTCAGGA CTCTACTCCC TCAGCAGCGT AGTGACCGTG CCCTCCAGCA GCTTGGGCAC | |
| 3061 | CCAGACCTAC ATCTGCAACG TGAATCACAA GCCCAGCAAC ACCAAGGTGG ACAAGAAAGT | |
| 3121 | TGAGCCCAAA TCTTGTGCGG CCGCACATCA TCATCACCAT CACGGGGCCG CAGAACAAAA | |
| 3181 | ACTCATCTCA GAAGAGGATC TGAATGGGGC CGCATAGACT GTTGAAAGTT GTTTAGCAAA | |
| 3241 | ACCTCATACA GAAAATTCAT TTACTAACGT CTGGAAAGAC GACAAAACTT TAGATCGTTA | |
| 3301 | CGCTAACTAT GAGGGCTGTC TGTGGAATGC TACAGGCGTT GTGGTTTGTA CTGGTGACGA | |
| 3361 | AACTCAGTGT TACGGTACAT GGGTTCCTAT TGGGCTTGCT ATCCCTGAAA ATGAGGGTGG | |
| 3421 | TGGCTCTGAG GGTGGCGGTT CTGAGGGTGG CGGTTCTGAG GGTGGCGGTA CTAAACCTCC | |
| 3481 | TGAGTACGGT GATACACCTA TTCCGGGCTA TACTTATATC AACCCTCTCG ACGGCACTTA | |
| 3541 | TCCGCCTGGT ACTGAGCAAA ACCCCGCTAA TCCTAATCCT TCTCTTGAGG AGTCTCAGCC | |
| 3601 | TCTTAATACT TTCATGTTTC AGAATAATAG GTTCCGAAAT AGGCAGGGTG CATTAACTGT | |
| 3661 | TTATACGGGC ACTGTTACTC AAGGCACTGA CCCCGTTAAA ACTTATTACC AGTACACTCC | |
| 3721 | TGTATCATCA AAAGCCATGT ATGACGCTTA CTGGAACGGT AAATTCAGAG ACTGCGCTTT | |
| 3781 | CCATTCTGGC TTTAATGAGG ATCCATTCGT TTGTGAATAT CAAGGCCAAT CGTCTGACCT | |
| 3841 | GCCTCAACCT CCTGTCAATG CTGGCGGCGG CTCTGGTGGT GGTTCTGGTG GCGGCTCTGA | |
| 3901 | GGGTGGCGGC TCTGAGGGTG GCGGTTCTGA GGGTGGCGGC TCTGAGGGTG GCGGTTCCGG | |
| 3961 | TGGCGGCTCC GGTTCCGGTG ATTTTGATTA TGAAAAAATG GCAAACGCTA ATAAGGGGGC | |
| 4021 | TATGACCGAA AATGCCGATG AAAACGCGCT ACAGTCTGAC GCTAAAGGCA AACTTGATTC | |
| 4081 | TGTCGCTACT GATTACGGTG CTGCTATCGA TGGTTTCATT GGTGACGTTT CCGGCCTTGC | |
| 4141 | TAATGGTAAT GGTGCTACTG GTGATTTTGC TGGCTCTAAT TCCCAAATGG CTCAAGTCGG | |
| 4201 | TGACGGTGAT AATTCACCTT TAATGAATAA TTTCCGTCAA TATTTACCTT CTTTGCCTCA | |
| 4261 | GTCGGTTGAA TGTCGCCCTT ATGTCTTTGG CGCTGGTAAA CCATATGAAT TTTCTATTGA | |
| 4321 | TTGTGACAAA ATAAACTTAT TCCGTGGTGT CTTTGCGTTT CTTTTATATG TTGCCACCTT | |
| 4381 | TATGTATGTA TTTTCGACGT TTGCTAACAT ACTGCGTAAT AAGGAGTCTT AATAAGAATT | |
| 4441 | CACTGGCCGT CGTTTTACAA CGTCGTGACT GGGAAAACCC TGGCGTTACC CAACTTAATC | |
| 4501 | GCCTTGCAGC ACATCCCCCT TTCGCCAGCT GGCGTAATAG CGAAGAGGCC CGCACCGATC | |
| 4561 | GCCCTTCCCA ACAGTTGCGC AGCCTGAATG GCGAATGGCG CCTGATGCGG TATTTTCTCC | |
| 4621 | TTACGCATCT GTGCGGTATT TCACACCGCA TATAAATTGT AAACGTTAAT ATTTTGTTAA | |
| 4681 | AATTCGCGTT AAATTTTTGT TAAATCAGCT CATTTTTTAA CCAATAGGCC GAAATCGGCA | |
| 4741 | AAATCCCTTA TAAATCAAAA GAATAGCCCG AGATAGGGTT GAGTGTTGTT CCAGTTTGGA | |
| 4801 | ACAAGAGTCC ACTATTAAAG AACGTGGACT CCAACGTCAA AGGGCGAAAA ACCGTCTATC | |
| 4861 | AGGGCGATGG CCCACTACGT GAACCATCAC CCAAATCAAG TTTTTTGGGG TCGAGGTGCC | |
| 4921 | GTAAAGCACT AAATCGGAAC CCTAAAGGGA GCCCCCGATT TAGAGCTTGA CGGGGAAAGC | |
| 4981 | CGGCGAACGT GGCGAGAAAG GAAGGGAAGA AAGCGAAAGG AGCGGGCGCT AGGGCGCTGG | |
| 5041 | CAAGTGTAGC GGTCACGCTG CGCGTAACCA CCACACCCGC CGCGCTTAAT GCGCCGCTAC | |
| 5101 | AGGGCGCGTA CTATGGTTGC TTTGACGGGT GCAGTCTCAG TACAATCTGC TCTGATGCCG | |
| 5161 | CATAGTTAAG CCAGCCCCGA CACCCGCCAA CACCCGCTGA CGCGCCCTGA CGGGCTTGTC | |
| 5221 | TGCTCCCGGC ATCCGCTTAC AGACAAGCTG TGACCGTCTC CGGGAGCTGC ATGTGTCAGA | |
| 5281 | GGTTTTCACC GTCATCACCG AAACGCGCGA | |

TABLE 8
Nucleotide sequence of pDY3F39

| | | |
|---|---|---|
| 1 | AATGCTACTA CTATTAGTAG AATTGATGCC ACCTTTTCAG CTCGCGCCCC AAATGAAAAT | (SEQ ID NO:10) |
| 61 | ATAGCTAAAC AGGTTATTGA CCATTTGCGA AATGTATCTA ATGGTCAAAC TAAATCTACT | |
| 121 | CGTTCGCAGA ATTGGGAATC AACTGTTATA TGGAATGAAA CTTCCAGACA CCGTACTTTA | |
| 181 | GTTGCATATT TAAAACATGT TGAGCTACAG CATTATATTC AGCAATTAAG CTCTAAGCCA | |
| 241 | TCCGCAAAAA TGACCTCTTA TCAAAAGGAG CAATTAAAGG TACTCTCTAA TCCTGACCTG | |
| 301 | TTGGAGTTTG CTTCCGGTCT GGTTCGCTTT GAAGCTCGAA TTAAAACGCG ATATTTGAAG | |
| 361 | TCTTTCGGGC TTCCTCTTAA TCTTTTTGAT GCAATCCGCT TTGCTTCTGA CTATAATAGT | |
| 421 | CAGGGTAAAG ACCTGATTTT TGATTTATGG TCATTCTCGT TTTCTGAACT GTTTAAAGCA | |
| 481 | TTTGAGGGGG ATTCAATGAA TATTTATGAC GATTCCGCAG TATTGGACGC TATCCAGTCT | |
| 541 | AAACATTTTA CTATTACCCC CTCTGGCAAA ACTTCTTTTG CAAAAGCCTC TCGCTATTTT | |
| 601 | GGTTTTTATC GTCGTCTGGT AAACGAGGGT TATGATAGTG TTGCTCTTAC TATGCCTCGT | |
| 661 | AATTCCTTTT GGCGTTATGT ATCTGCATTA GTTGAATGTG GTATTCCTAA ATCTCAACTG | |
| 721 | ATGAATCTTT CTACCTGTAA TAATGTTGTT CCGTTAGTTC GTTTTATTAA CGTAGATTTT | |
| 781 | TCTTCCCAAC GTCCTGACTG GTATAATGAG CCAGTTCTTA AAATCGCATA AGGTAATTCA | |
| 841 | CAATGATTAA AGTTGAAATT AAACCATCTC AAGCCCAATT TACTACTCGT TCTGGTGTTT | |
| 901 | CTCGTCAGGG CAAGCCTTAT TCACTGAATG AGCAGCTTTG TTACGTTGAT TTGGGTAATG | |
| 961 | AATATCCGGT TCTTGTCAAG ATTACTCTTG ATGAAGGTCA GCCAGCCTAT GCGCCTGGTC | |
| 1021 | TGTACACCGT TCATCTGTCC TCTTTCAAAG TTGGTCAGTT CGGTTCCCTT ATGATTGACC | |
| 1081 | GTCTGCGCCT CGTTCCGGCT AAGTAACATG GAGCAGGTCG CGGATTTCGA CACAATTTAT | |
| 1141 | CAGGCGATGA TACAAATCTC CGTTGTACTT TGTTTCGCGC TTGGTATAAT CGCTGGGGGT | |
| 1201 | CAAAGATGAG TGTTTTAGTG TATTCTTTTG CCTCTTTCGT TTTAGGTTGG TGCCTTCGTA | |
| 1261 | GTGGCATTAC GTATTTTACC CGTTTAATGG AAACTTCCTC ATGAAAAAGT CTTTAGTCCT | |
| 1321 | CAAAGCCTCT GTAGCCGTTG CTACCCTCGT TCCGATGCTG TCTTTCGCTG CTGAGGGTGA | |
| 1381 | CGATCCCGCA AAAGCGGCCT TTAACTCCCT GCAAGCCTCA GCGACCGAAT ATATCGGTTA | |
| 1441 | TGCGTGGGCG ATGGTTGTTG TCATTGTCGG CGCAACTATC GGTATCAAGC TGTTTAAGAA | |
| 1501 | ATTCACCTCG AAAGCAAGCT GATAAACCGA TACAATTAAA GGCTCCTTTT GGAGCCTTTT | |
| 1561 | TTTTTGGAGA TTTTCAACGT GAAAAAATTA TTATTCGCAA TTCCTTTAGT TGTTCCTTTC | |
| 1621 | TATTCTGGCG CGGCCGAATC ACATCTAGAC GGCGCCGCTG AAACTGTTGA AAGTTGTTTA | |
| 1681 | GCAAAATCCC ATACAGAAAA TTCATTTACT AACGTCTGGA AAGACGACAA AACTTTAGAT | |
| 1741 | CGTTACGCTA ACTATGAGGG CTGTCTGTGG AATGCTACAG GCGTTGTAGT TTGTACTGGT | |
| 1801 | GACGAAACTC AGTGTTACGG TACATGGGTT CCTATTGGGC TTGCTATCCC TGAAAATGAG | |
| 1861 | GGTGGTGGCT CTGAGGGTGG CGGTTCTGAG GGTGGCGGTT CTGAGGGTGG CGGTACTAAA | |
| 1921 | CCTCCTGAGT ACGGTGATAC ACCTATTCCG GGCTATACTT ATATCAACCC TCTCGACGGC | |
| 1981 | ACTTATCCGC CTGGTACTGA GCAAAACCCC GCTAATCCTA ATCCTTCTCT TGAGGAGTCT | |
| 2041 | CAGCCTCTTA ATACTTTCAT GTTTCAGAAT AATAGGTTCC GAAATAGGCA GGGGGCATTA | |
| 2101 | ACTGTTTATA CGGGCACTGT TACTCAAGGC ACTGACCCCG TTAAAACTTA TTACCAGTAC | |
| 2161 | ACTCCTGTAT CATCAAAAGC CATGTATGAC GCTTACTGGA ACGGTAAATT CAGAGACTGC | |
| 2221 | GCTTTCCATT CTGGCTTTAA TGAGGATTTA TTTGTTTGTG AATATCAAGG CCAATCGTCT | |
| 2281 | GACCTGCCTC AACCTCCTGT CAATGCTGGC GGCGGCTCTG GTGGTGGTTC TGGTGGCGGC | |
| 2341 | TCTGAGGGTG GTGGCTCTGA GGGAGGCGGT TCCGGTGGTG GCTCTGGTTC CGGTGATTTT | |
| 2401 | GATTATGAAA AGATGGCAAA CGCTAATAAG GGGGCTATGA CCGAAAATGC CGATGAAAAC | |
| 2461 | GCGCTACAGT CTGACGCTAA AGGCAAACTT GATTCTGTCG CTACTGATTA CGGTGCTGCT | |
| 2521 | ATCGATGGTT TCATTGGTGA CGTTTCCGGC CTTGCTAATG GTAATGGTGC TACTGGTGAT | |
| 2581 | TTTGCTGGCT CTAATTCCCA AATGGCTCAA GTCGGTGACG GTGATAATTC ACCTTTAATG | |
| 2641 | AATAATTTCC GTCAATATTT ACCTTCCCTC CCTCAATCGG TTGAATGTCG CCCTTTTGTC | |
| 2701 | TTTGGCGCTG GTAAACCATA TGAATTTTCT ATTGATTGTG ACAAAATAAA CTTATTCCGT | |
| 2761 | GGTGTCTTTG CGTTTCTTTT ATATGTTGCC ACCTTTATGT ATGTATTTTC TACGTTTGCT | |
| 2821 | AACATACTGC GTAATAAGGA GTCTTAATCA TGCCAGTTCT TTTGGGTATT CCGTTATTAT | |
| 2881 | TGCGTTTCCT CGGTTTCCTT CTGGTAACTT TGTTCGGCTA TCTGCTTACT TTTCTTAAAA | |
| 2941 | AGGGCTTCGG TAAGATAGCT ATTGCTATTT CATTGTTTCT TGCTCTTATT ATTGGGCTTA | |
| 3001 | ACTCAATTCT TGTGGGTTAT CTCTCTGATA TTAGCGCTCA ATTACCCTCT GACTTTGTTC | |
| 3061 | AGGGTGTTCA GTTAATTCTC CCGTCTAATG CGCTTCCCTG TTTTTATGTT ATTCTCTCTG | |
| 3121 | TAAAGGCTGC TATTTTCATT TTTGACGTTA AACAAAAAAT CGTTTCTTAT TTGGATTGGG | |
| 3181 | ATAAATAATA TGGCTGTTTA TTTTGTAACT GGCAAATTAG GCTCTGGAAA GACGCTCGTT | |
| 3241 | AGCGTTGGTA AGATTCAGGA TAAAATTGTA GCTGGGTGCA AAATAGCAAC TAATCTTGAT | |
| 3301 | TTAAGGCTTC AAAACCTCCC GCAAGTCGGG AGGTTCGCTA AAACGCCTCG CGTTCTTAGA | |
| 3361 | ATACCGGATA AGCCTTCTAT ATCTGATTTG CTTGCTATTG GGCGCGGTAA TGATTCCTAC | |
| 3421 | GATGAAAATA AAAACGGCTT GCTTGTTCTC GATGAGTGCG GTACTTGGTT TAATACCCGT | |
| 3481 | TCTTGGAATG ATAAGGAAAG ACAGCCGATT ATTGATTGGT TTCTACATGC TCGTAAATTA | |
| 3541 | GGATGGGATA TTATTTTTCT TGTTCAGGAC TTATCTATTG TTGATAAACA GGCGCGTTCT | |
| 3601 | GCATTAGCTG AACATGTTGT TTATTGTCGT CGTCTGGACA GAATTACTTT ACCTTTTGTC | |
| 3661 | GGTACTTTAT ATTCTCTTAT TACTGGCTCG AAAATGCCTC TGCCTAAATT ACATGTTGGC | |
| 3721 | GTTGTTAAAT ATGGCGATTC TCAATTAAGC CCTACTGTTG AGCGTTGGCT TTATACTGGT | |
| 3781 | AAGAATTTGT ATAACGCATA TGATACTAAA CAGGCTTTTT CTAGTAATTA TGATTCCGGT | |
| 3841 | GTTTATTCTT ATTTAACGCC TTATTTATCA CACGGTCGGT ATTTCAAACC ATTAAATTTA | |
| 3901 | GGTCAGAAGA TGAAATTAAC TAAAATATAT TTGAAAAAGT TTTCTCGCGT TCTTTGTCTT | |
| 3961 | GCGATTGGAT TTGCATCAGC ATTTACATAT AGTTATATAA CCCAACCTAA GCCGGAGGTT | |
| 4021 | AAAAAGGTAG TCTCTCAGAC CTATGATTTT GATAAATTCA CTATTGACTC TTCTCAGCGT | |
| 4081 | CTTAATCTAA GCTATCGCTA TGTTTTCAAG GATTCTAAGG GAAAATTAAT TAATAGCGAC | |
| 4141 | GATTTACAGA AGCAAGGTTA TTCACTCACA TATATTGATT TATGTACTGT TTCCATTAAA | |
| 4201 | AAAGGTAATT CAAATGAAAT TGTTAAATGT AATTAATTTT GTTTTCTTGA TGTTTGTTTC | |
| 4261 | ATCATCTTCT TTTGCTCAGG TAATTGAAAT GAATAATTCG CCTCTGCGCG ATTTTGTAAC | |
| 4321 | TTGGTATTCA AAGCAATCAG GCGAATCCGT TATTGTTTCT CCCGATGTAA AAGGTACTGT | |
| 4381 | TACTGTATAT TCATCTGACG TTAAACCTGA AAATCTACGC AATTTCTTTA TTTCTGTTTT | |
| 4441 | ACGTGCAAAT AATTTTGATA TGGTAGGTTC TAACCCTTCC ATTATTOAGA AGTATAATCC | |
| 4501 | AAACAATCAG GATTATATTG ATGAATTGCC ATCATCTGAT AATCAGGAAT ATGATGATAA | |
| 4561 | TTCCGCTCCT TCTGGTGGTT TCTTTGTTCC GCAAAATGAT AATGTTACTC AAACTTTTAA | |
| 4621 | AATTAATAAC GTTCGGGCAA AGGATTTAAT ACGAGTTGTC GAATTGTTTG TAAAGTCTAA | |
| 4681 | TACTTCTAAA TCCTCAAATG TATTATCTAT TGACGGCTCT AATCTATTAG TTGTTAGTGC | |
| 4741 | TCCTAAAGAT ATTTTAGATA ACCTTCCTCA ATTCCTTTCA ACTGTTGATT TGCCAACTGA | |
| 4801 | CCAGATATTG ATTGAGGGTT TGATATTTGA GGTTCAGCAA GGTGATGCTT TAGATTTTTC | |
| 4861 | ATTTGCTGCT GGCTCTCAGC GTGGCACTGT TGCAGGCGGT GTTAATACTG ACCGCCTCAC | |
| 4921 | CTCTGTTTTA TCTTCTGCTG GTGGTTCGTT CGGTATTTTT AATGGCGATG TTTTAGGGCT | |
| 4981 | ATCAGTTCGC GCATTAAAGA CTAATAGCCA TTCAAAAATA TTGTCTGTGC CACGTATTCT | |
| 5041 | TACGCTTTCA GGTCAGAAGG GTTCTATCTC TGTTGGCCAG AATGTCCCTT TTATTACTGG | |
| 5101 | TCGTGTGACT GGTGAATCTG CCAATGTAAA TAATCCATTT CAGACGATTG AGCGTCAAAA | |
| 5161 | TGTAGGTATT TCCATGAGCG TTTTTCCTGT TGCAATGGCT GGCGGTAATA TTGTTCTGGA | |
| 5221 | TATTACCAGC AAGGCCGATA GTTTGAGTTC TTCTACTCAG GCAAGTGATG TTATTACTAA | |
| 5281 | TCAAAGAAGT ATTGCTACAA CGGTTAATTT GCGTGATGGA CAGACTCTTT TACTCGGTGG | |
| 5341 | CCTCACTGAT TATAAAAACA CTTCTCAGGA TTCTGGCGTA CCGTTCCTGT CTAAAATCCC | |
| 5401 | TTTAATCGGC CTCCTGTTTA GCTCCCGCTC TGATTCTAAC GAGGAAAGCA CGTTATACGT | |
| 5461 | GCTCGTCAAA GCAACCATAG TACGCGCCCT GTAGCGGCGC ATTAAGCGCG GCGGGTGTGG | |
| 5521 | TGGTTACGCG CAGCGTGACC GCTACACTTG CCAGCGCCCT AGCGCCCGCT CCTTTCGCTT | |
| 5581 | TCTTCCCTTC CTTTCTCGCC ACGTTCGCCG GCTTTCCCCG TCAAGCTCTA AATCGGGGGC | |
| 5641 | TCCCTTTAGG GTTCCGATTT AGTGCTTTAC GGCACCTCGA CCCCAAAAAA CTTGATTTGG | |
| 5701 | GTGATGGTTC ACGTAGTGGG CCATCGCCCT GATAGACGGT TTTTCGCCCT TTGACGTTGG | |
| 5761 | AGTCCACGTT CTTTAATAGT GGACTCTTGT TCCAAACTGG AACAACACTC AACCCTATCT | |
| 5821 | CGGGCTATTC TTTTGATTTA TAAGGGATTT TGCCGATTTC GGAACCACCA TCAAACAGGA | |
| 5881 | TTTTCGCCTG CTGGGGCAAA CCAGCGTGGA CCGCTTGCTG CAACTCTCTC AGGGCCAGGC | |
| 5941 | GGTGAAGGGC AATCAGCTGT TGCCCGTCTC ACTGGTGAAA AGAAAAACCA CCCTGGATCC | |
| 6001 | AAGCTTGCAG GTGGCACTTT TCGGGGAAAT GTGCGCGGAA CCCCTATTTG TTTATTTTTC | |
| 6061 | TAAATACATT CAAATATGTA TCCGCTCATG AGACAATAAC CCTGATAAAT GCTTCAATAA | |
| 6121 | TATTGAAAAA GGAAGAGTAT GAGTATTCAA CATTTCCGTG TCGCCCTTAT TCCCTTTTTT | |
| 6181 | GCGGCATTTT GCCTTCCTGT TTTTGCTCAC CCAGAAACGC TGGTGAAAGT AAAAGATGCT | |
| 6241 | GAAGATCAGT TGGGCGCACT AGTGGGTTAC ATCGAACTGG ATCTCAACAG CGGTAAGATC | |
| 6301 | CTTGAGAGTT TTCGCCCCGA AGAACGTTTT CCAATGATGA GCACTTTTAA AGTTCTGCTA | |
| 6361 | TGTGGCGCGG TATTATCCCG TATTGACGCC GGGCAAGAGC AACTCGGTCG CCGCATACAC | |
| 6421 | TATTCTCAGA ATGACTTGGT TGAGTACTCA CCAGTCACAG AAAAGCATCT TACGGATGGC | |
| 6481 | ATGACAGTAA GAGAATTATG CAGTGCTGCC ATAACCATGA GTGATAACAC TGCGGCCAAC | |
| 6541 | TTACTTCTGA CAACGATCGG AGGACCGAAG GAGCTAACCG CTTTTTTGCA CAACATGGGG | |
| 6601 | GATCATGTAA CTCGCCTTGA TCGTTGGGAA CCGGAGCTGA ATGAAGCCAT ACCAAACGAC | |
| 6661 | GAGCGTGACA CCACGATGCC TGTAGCAATG GCAACAACGT TGCGCAAACT ATTAACTGGC | |
| 6721 | GAACTACTTA CTCTAGCTTC CCGGCAACAA TTAATAGACT GGATGGAGGC GGATAAAGTT | |
| 6781 | GCAGGACCAC TTCTGCGCTC GGCCCTTCCG GCTGGCTGGT TTATTGCTGA TAAATCTGGA | |
| 6841 | GCCGGTGAGC GTGGGTCTCG CGGTATCATT GCAGCACTGG GGCCAGATGG TAAGCCCTCC | |
| 6901 | CGTATCGTAG TTATCTACAC GACGGGGAGT CAGGCAACTA TGGATGAACG AAATAGACAG | |
| 6961 | ATCGCTGAGA TAGGTGCCTC ACTGATTAAG CATTGGTAAC TGTCAGACCA AGTTTACTCA | |
| 7021 | TATATACTTT AGATTGATTT AAAACTTCAT TTTTAATTTA AAAGGATCTA GGTGAAGATC | |
| 7081 | CTTTTTGATA ATCTCATGAC CAAAATCCCT TAACGTGAGT TTTCGTTCCA CTGTACGTAA | |
| 7141 | GACCCCCAAG CTTGTCGACT GAATGGCGAA TGGCGCTTTG CCTGGTTTCC GGCACCAGAA | |
| 7201 | GCGGTGCCGG AAAGCTGGCT GGAGTGCGAT CTTCCTGACG CTCGAGCGCA ACGCAATTAA | |
| 7261 | TGTGAGTTAG CTCACTCATT AGGCACCCCA GGCTTTACAC TTTATGCTTC CGGCTCGTAT | |
| 7321 | GTTGTGTGGA ATTGTGAGCG GATAACAATT TCACACAGGA AACAGCTATG ACCATGATTA | |
| 7381 | CGCCAAGCTT TGGAGCCTTT TTTTTGGAGA TTTTCAACGT GAAAAAATTA TTATTCGCAA | |
| 7441 | TTCCTTTAGT TGTTCCTTTC TATTCTCACA GTGCACAGTG ATAGACTAGT TAGACGCGTG | |
| 7501 | CTTAAAGGCC TCCAATCCTC TTGGCGCGCC AATTCTATTT CAAGGAGACA GTCATAATGA | |
| 7561 | AATACCTATT GCCTACGGCA GCCGCTGGAT TGTTATTACT CGCGGCCCAG CCGGCCCTCT | |
| 7621 | GATAAGATAT CACTTGTTTA AACTCTGCTT GGCCCTCTTG GCCTTCTAGT AGACTTGCGG | |
| 7681 | CCGCACATCA TCATCACCAT CACGGGGCCG CAGAACAAAA ACTCATCTCA GAAGAGGATC | |
| 7741 | TGAATGGGGC CGCATAGGCT AGCGATATCA ACGATGATCG TATGGCTTCT ACTGCCGAGA | |
| 7801 | CAGTCGAATC CTGCCTGGCC AAGCCTCACA CTGAGAATAG TTTCACAAAT GTGTGGAAGG | |
| 7861 | ATGATAAGAC CCTTGATCGA TATGCCAATT ACGAAGGCTG CTTATGGAAT GCCACCGGCG | |
| 7921 | TCGTTGTCTG CACGGGCGAT GAGACACAAT GCTATGGCAC GTGGGTGCCG ATAGGCTTAG | |
| 7981 | CCATACCGGA GAACGAAGGC GGCGGTAGCG AAGGCGGTGG CAGCGAAGGC GGTGGATCCG | |
| 8041 | AAGGAGGTGG AACCAAGCCG CCGGAATATG GCGACACTCC GATACCTGGT TACACCTACA | |
| 8101 | TTAATCCGTT AGATGGAACC TACCCTCCGG GCACCGAACA GAATCCTGCC AACCCGAACC | |
| 8161 | CAAGCTTAGA AGAAAGCCAA CCGTTAAACA CCTTTATGTT CCAAAACAAC CGTTTTAGGA | |
| 8221 | ACCGTCAAGG TGCTCTTACC GTGTACACTG GAACCGTCAC CCAGGGTACC GATCCTGTCA | |
| 8281 | AGACCTACTA TCAATATACC CCGGTCTCGA GTAAGGCTAT GTACGATGCC TATTGGAATG | |
| 8341 | GCAAGTTTCG TGATTGTGCC TTTCACAGCG GTTTCAACGA AGACCCTTTT GTCTGCGAGT | |
| 8401 | ACCAGGGTCA GAGTAGCGAT TTACCGCAGC CACCGGTTAA CGCGGGTGGT GGTAGCGGCG | |
| 8461 | GAGGCAGCGG CGGTGGTAGC GAAGGCGGAG GTAGCGAAGG AGGTGGCAGC GGAGGCGGTA | |
| 8521 | GCGGCAGTGG CGACTTCGAC TACGAGAAAA TGGCTAATGC CAACAAAGGC GCCATGACTG | |
| 8581 | AGAACGCTGA CGAGAATGCA CTGCAAAGTG ATGCCAAGGG TAAGTTAGAC AGCGTCGCCA | |
| 8641 | CAGACTATGG TGCTGCCATC GACGGCTTTA TCGGCGATGT CAGTGGTCTG GCTAACGGCA | |
| 8701 | ACGGAGCCAC CGGAGACTTC GCAGGTTCGA ATTCTCAGAT GGCCCAGGTT GGAGATGGGG | |
| 8761 | ACAACAGTCC GCTTATGAAC AACTTTAGAC AGTACCTTCC GTCTCTTCCG CAGAGTGTCG | |
| 8821 | AGTGCCGTCC ATTCGTTTTC TCTGCCGGCA AGCCTTACGA GTTCAGCATC GACTGCGATA | |
| 8881 | AGATCAATCT TTTCCGCGGC GTTTTCGCTT TCTTGCTATA CGTCGCTACT TTCATGTACG | |
| 8941 | TTTTCAGCAC TTTCGCCAAT ATTTTACGCA ACAAAGAAAG CTAGTGATCT CCTAGGAAGC | |
| 9001 | CCGCCTAATG AGCGGGCTTT TTTTTTCTGG TATGCATCCT GAGGCCGATA CTGTCGTCGT | |
| 9061 | CCCCTCAAAC TGGCAGATGC ACGGTTACGA TGCGCCCATC TACACCAACG TGACCTATCC | |
| 9121 | CATTACGGTC AATCCGCCGT TTGTTCCCAC GGAGAATCCG ACGGGTTGTT ACTCGCTCAC | |
| 9181 | ATTTAATGTT GATGAAAGCT GGCTACAGGA AGGCCAGACG CGAATTATTT TTGATGGCGT | |
| 9241 | TCCTATTGGT TAAAAAATGA GCTGATTTAA CAAAAATTTA ATGCGAATTT TAACAAAATA | |
| 9301 | TTAACGTTTA CAATTTAAAT ATTTGCTTAT ACAATCTTCC TGTTTTTGGG GCTTTTCTGA | |
| 9361 | TTATCAACCG GGGTACATAT GATTGACATG CTAGTTTTAC GATTACCGTT CATCGATTCT | |
| 9421 | CTTGTTTGCT CCAGACTCTC AGGCAATGAC CTGATAGCCT TTGTAGATCT CTCAAAAATA | |
| 9481 | GCTACCCTCT CCGGCATTAA TTTATCAGCT AGAACGGTTG AATATCATAT TGATGGTGAT | |
| 9541 | TTGACTGTCT CCGGCCTTTC TCACCCTTTT GAATCTTTAC CTACACATTA CTCAGGCATT | |
| 9601 | GCATTTAAAA TATATGAGGG TTCTAAAAAT TTTTATCCTT GCGTTGAAAT AAAGGCTTCT | |
| 9661 | CCCGCAAAAG TATTACAGGG TCATAATGTT TTTGGTACAA CCGATTTAGC TTTATGCTCT | |
| 9721 | GAGGCTTTAT TGCTTAATTT TGCTAATTCT TTGCCTTGCC TGTATGATTT ATTGGATGTT | |

TABLE 9
Nucleotide sequence of pRH06.

| | |
|---|---|
| TTAATAGCGACGATTTACAGAAGCAAGGTTATTCACTCACATATATTGATTTATGTACTGTTTCCATTAAAAAAGGT | (SEQ ID NO:11) |
| AATTCAAATGAAATTGTTAAATGTAATTAATTTTGTTTTCTTGATGTTTGTTTCATCATCTTCTTTTGCTCAGGTAA | |
| TTGAAATGAATAATTCGCCTCTGCGCGATTTTGTAACTTGGTATTCAAAGCAATCAGGCGAATCCGTTATTGTTTCT | |
| CCCGATGTAAAAGGTACTGTTACTGTATATTCATCTGACGTTAAACCTGAAAATCTACGCAATTTCTTTATTTCTGT | |
| TTTACGTGCAAATAATTTTGATATGGTAGGTTCTAACCCTTCCATTATTCAGAAGTATAATCCAAACAATCAGGATT | |
| ATATTGATGAATTGCCATCATCTGATAATCAGGAATATGATGATAATTCCGCTCCTTCTGGTGGTTTCTTTGTTCCG | |
| CAAAATGATAATGTTACTCAAACTTTTAAAATTAATAACGTTCGGGCAAAGGATTTAATACGAGTTGTCGAATTGTT | |
| TGTAAAGTCTAATACTTCTAAATCCTCAAATGTATTATCTATTGACGGCTCTAATCTATTAGTTGTTAGTGCTCCTA | |
| AAGATATTTTAGATAACCTTCCTCAATTCCTTTCAACTGTTGATTTGCCAACTGACCAGATATTGATTGAGGGTTTG | |
| ATATTTGAGGTTCAGCAAGGTGATGCTTTAGATTTTTCATTTGCTGCTGGCTCTCAGCGTGGCACTGTTGCAGGCGG | |
| TGTTAATACTGACCGCCTCACCTCTGTTTTATCTTCTGCTGGTGGTTCGTTCGGTATTTTTAATGGCGATGTTTTAG | |
| GGCTATCAGTTCGCGCATTAPAGACTAATAGCCATTCAAAAATATTGTCTGTGCCACGTATTCTTACGCTTTCAGGT | |
| CAGAAGGGTTCTATCTCTGTTGGCCAGAATGTCCCTTTTATTACTGGTCGTGTGACTGGTGAATCTGCCAATGTAAA | |
| TAATCCATTTCAGACGATTGAGCGTCAAAATGTAGGTATTTCCATGAGCGTTTTTCCTGTTGCAATGGCTGGCGGTA | |
| ATATTGTTCTGGATATTACCAGCAAGGCCGATAGTTTGAGTTCTTCTACTCAGGCAAGTGATGTTATTACTAATCAA | |
| AGAAGTATTGCTACAACGGTTAATTTGCGTGATGGACAGACTCTTTTACTCGGTGGCCTCACTGATTATAAAAACAC | |
| TTCTCAGGATTCTGGCGTACCGTTCCTGTCTAAAATCCCTTTAATCGGCCTCCTGTTTAGCTCCCGCTCTGATTCTA | |
| ACGAGGAAAGCACGTTATACGTGCTCGTCAAAGCAACCATAGTACGCGCCCTGTAGCGGCGCATTAAGCGCGGCGGG | |
| TGTGGTGGTTACGCGCAGCGTGACCGCTACACTTGCCAGCGCCCTAGCGCCCGCTCCTTTCGCTTTCTTCCCTTCCT | |
| TTCTCGCCACGTTCGCCGGCTTTCCCCGTCAAGCTCTAAATCGGGGGCTCCCTTTAGGGTTCCGATTTAGTGCTTTA | |
| CGGCACCTCGACCCCAAAAAACTTGATTTGGGTGATGGTTCACGTAGTGGGCCATCGCCCTGATAGACGGTTTTTCG | |
| CCCTTTGACGTTGGAGTCCACGTTCTTTAATAGTGGACTCTTGTTCCAAACTGGAACAACACTCAACCCTATCTCGG | |
| GCTATTCTTTTGATTTATAAGGGATTTTGCCGATTTCGGAACCACCATCAAACAGGATTTTCGCCTGCTGGGGCAAA | |
| CCAGCGTGGACCGCTTGCTGCAACTCTCTCAGGGCCAGGCGGTGAAGGGCAATCAGCTGTTGCCCGTCTCACTGGTG | |
| AAAAGAAAAACCACCCTGGATCCAAGCTTGCAGGTGGCACTTTTCGGGGAAATGTGCGCGGAACCCCTATTTGTTTA | |
| TTTTTCTAAATACATTCAAATATGTATCCGCTCATGAGACAATAACCCTGATAAATGCTTCAATAATATTGAAAAAG | |
| GAAGAGTATGAGTATTCAACATTTCCGTGTCGCCCTTATTCCCTTTTTTGCGGCATTTTGCCTTCCTGTTTTTGCTC | |
| ACCCAGAAACGCTGGTGAAAGTAAAAGATGCTGAAGATCAGTTGGGCGCACTAGTGGGTTACATCGAACTGGATCTC | |
| AACAGCGGTAAGATCCTTGAGAGTTTTCGCCCCGAAGAACGTTTTCCAATGATGAGCACTTTTAAAGTTCTGCTATG | |
| TGGCGCGGTATTATCCCGTATTGACGCCGGGCAAGAGCAACTCGGTCGCCGCATACACTATTCTCAGAATGACTTGG | |
| TTGAGTACTCACCAGTCACAGAAAAGCATCTTACGGATGGCATGACAGTAAGAGAATTATGCAGTGCTGCCATAACC | |
| ATGAGTGATAACACTGCGGCCAACTTACTTCTGACAACGATCGGAGGACCGAAGGAGCTAACCGCTTTTTTGCACAA | |
| CATGGGGGATCATGTAACTCGCCTTGATCGTTGGGAACCGGAGCTGAATGAAGCCATACCAAACGACGAGCGTGACA | |
| CCACGATGCCTGTAGCAATGGCAACAACGTTGCGCAAACTATTAACTGGCGAACTACTTACTCTAGCTTCCCGGCAA | |
| CAATTAATAGACTGGATGGAGGCGGATAAAGTTGCAGGACCACTTCTGCGCTCGGCCCTTCCGGCTGGCTGGTTTAT | |
| TGCTGATAAATCTGGAGCCGGTGAGCGTGGGTCTCGCGGTATCATTGCAGCACTGGGGCCAGATGGTAAGCCCTCCC | |
| GTATCGTAGTTATCTACACGACGGGGAGTCAGGCAACTATGGATGAACGAAATAGACAGATCGCTGAGATAGGTGCC | |
| TCACTGATTAAGCATTGGTAACTGTCAGACCAAGTTTACTCATATATACTTTAGATTGATTTAAAACTTCATTTTTA | |
| ATTTAAAAGGATCTAGGTGAAGATCCTTTTTGATAATCTCATGACCAAAATCCCTTAACGTGAGTTTTCGTTCCACT | |
| GTACGTAAGACCCCCAAGCTTGTCGACCGCAACGCAATTAATGTGAGTTAGCTCACTCATTAGGCACCCCAGGCTTT | |
| ACACTTTATGCTTCCGGCTCGTATGTTGTGTGGAATTGTGAGCGGATAACAATTTCACCCATGCTTTGGACAGGAAA | |
| CAGCTATGAAAAAGCTTTTATTCGCTATCCCGTTAGTTGTACCGTTCTATTCTCACTCTGCCGAGACAGTCGAATCC | |
| TGCCTGGCCAAGTCTCACACTGAGAATAGTTTCACAAATGTGTGGAAGGATGATAAGACCCTTGATCGATATGCCAA | |
| TTACGAAGGCTGCTTATGGAATGCCACCGGCGTCGTTGTCTGCACGGGCGATGAGACACAATGCTATGGCACGTGGG | |
| TGCCGATAGGCTTAGCCATACCGGAGAACGAAGGCGGCGGTAGCGAAGGCGGTGGCAGCGAAGGCGGTGGATCCGAA | |
| GGAGGTGGAACCAAGCCGCCGGAATATGGCGACACTCCGATACCTGGTTACACCTACATTAATCCGTTAGATGGAAC | |
| CTACCCTCCGGGCACCGAACAGAATCCTGCCAACCCGAACCCAAGCTTAGAAGAAAGCCAACCGTTAAACACCTTTA | |
| TGTTCCAAAACAACCGTTTTAGGAACCGTCAAGGTGCTCTTACCGTGTACACTGGAACCGTCACCCAGGGTACCGAT | |
| CCTGTCAAGACCTACTATCAATATACCCCGGTCTCGAGTAAGGCTATGTACGATGCCTATTGGAATGGCAAGTTTCG | |
| TGATTGTGCCTTTCACAGCGGTTTCAACGAAGACCCTTTTGTCTGCGAGTACCAGGGTCAGAGTAGCGATTTACCGC | |
| AGCCACCGGTTAACGCGGGTGGTGGTAGCGGCGGAGGCAGCGGCGGTGGTAGCGAAGGCGGAGGTAGCGAAGGAGGT | |
| GGCAGCGGAGGCGGTAGCGGCAGTGGCGACTTCGACTACGAGAAAATGGCTAATGCCAACAAAGGCGCCATGACTGA | |
| GAACGCTGACGAGAATGCACTGCAAAGTGATGCCAAGGGTAAGTTAGACAGCGTCGCCACAGACTATGGTGCTGCCA | |
| TCGACGGCTTTATCGGCGATGTCAGTGGTCTGGCTAACGGCAACGGAGCCACCGGAGACTTCGCAGGTTCGAATTCT | |
| CAGATGGCCCAGGTTGGAGATGGGGACAACAGTCCGCTTATGAACAACTTTAGACAGTACCTTCCGTCTCTTCCGCA | |
| GAGTGTCGAGTGCCGTCCATTCGTTTTCGGAGCCGGCAAGCCTTACGAGTTCAGCATCGACTGCGATAAGATCAATC | |
| TTTTCCGCGGCGTTTTCGCTTTCTTGCTATACGTCGCTACTTTCATGTACGTTTTCAGCACTTTCGCCAATATTTTA | |
| CGCAACAAAGAAAGCTAGTGATCTCCTAGGAAGCCCGCCTAATGAGCGGGCTTTTTTTTTCTGGTATGCATCCTGAG | |
| GCCGATACTGTCGTCGTCCCCTCAAACTGGCAGATGCACGGTTACGATGCGCCCATCTACACCAACGTGACCTATCC | |
| CATTACGGTCAATCCGCCGTTTGTTCCCACGGAGAATCCGACGGGTTGTTACTCGCTCACATTTAATGTTGATGAAA | |
| GCTGGCTACAGGAAGGCCAGACGCGAATTATTTTTGATGGCGTTCCTATTGGTTAAAAAATGAGCTGATTTAACAAA | |
| AATTTAATGCGAATTTTAACAAAATATTAACGTTTACAATTTAAATATTTGCTTATACAATCTTCCTGTTTTTGGGG | |
| CTTTTCTGATTATCAACCGGGGTACATATGATTGACATGCTAGTTTTACGATTACCGTTCATCGATTCTCTTGTTTG | |
| CTCCAGACTCTCAGGCAATGACCTGATAGCCTTTGTAGATCTCTCAAAAATAGCTACCCTCTCCGGCATGAATTTAT | |
| CAGCTAGAACGGTTGAATATCATATTGATGGTGATTTGACTGTCTCCGGCCTTTCTCACCCTTTTGAATCTTTACCT | |
| ACACATTACTCAGGCATTGCATTTAAAATATATGAGGGTTCTAAAAATTTTTATCCTTGCGTTGAAATAAAGGCTTC | |
| TCCCGCAAAAGTATTACAGGGTCATAATGTTTTTGGTACAACCGATTTAGCTTTATGCTCTGAGGCTTTATTGCTTA | |
| ATTTTGCTAATTCTTTGCCTTGCCTGTATGATTTATTGGATGTTAATGCTACTACTATTAGTAGAATTGATGCCACC | |
| TTTTCAGCTCGCGCCCCAAATGAAAATATAGCTAAACAGGTTATTGACCATTTGCGAAATGTATCTAATGGTCAAAC | |
| TAAATCTACTCGTTCGCAGAATTGGGAATCAACTGTTACATGGAATGAAACTTCCAGACACCGTACTTTAGTTGCAT | |
| ATTTAAAACATGTTGAGCTACAGCACCAGATTCAGCAATTAAGCTCTAAGCCATCCGCAAAAATGACCTCTTATCAA | |
| AAGGAGCAATTAAAGGTACTCTCTAATCCTGACCTGTTGGAGTTTGCTTCCGGTCTGGTTCGCTTTGAAGCTCGAAT | |
| TAAAACGCGATATTTGAAGTCTTTCGGGCTTCCTCTTAATCTTTTTGATGCAATCCGCTTTGCTTCTGACTATAATA | |
| GTCAGGGTAAAGACCTGATTTTTGATTTATGGTCATTCTCGTTTTCTGAACTGTTTAAAGCATTTGAGGGGGATTCA | |
| ATGAATATTTATGACGATTCCGCAGTATTGGACGCTATCCAGTCTAAACATTTTACTATTACCCCCTCTGGCAAAAC | |
| TTCTTTTGCAAAAGCCTCTCGCTATTTTGGTTTTTATCGTCGTCTGGTAAACGAGGGTTATGATAGTGTTGCTCTTA | |
| CTATGCCTCGTAATTCCTTTTGGCGTTATGTATCTGCATTAGTTGAATGTGGTATTCCTAAATCTCAACTGATGAAT | |
| CTTTCTACCTGTAATAATGTTGTTCCGTTAGTTCGTTTTATTAACGTAGATTTTTCTTCCCAACGTCCTGACTGGTA | |
| TAATGAGCCAGTTCTTAAAATCGCATAAGGTAATTCACAATGATTAAAGTTGAAATTAAACCATCTCAAGCCCAATT | |
| TACTACTCGTTCTGGTGTTTCTCGTCAGGGCAAGCCTTATTCACTGAATGAGCAGCTTTGTTACGTTGATTTGGGTA | |
| ATGAATATCCGGTTCTTGTCAAGATTACTCTTGATGAAGGTCAGCCAGCCTATGCGCCTGGTCTGTACACCGTTCAT | |
| CTGTCCTCTTTCAAAGTTGGTCAGTTCGGTTCCCTTATGATTGACCGTCTGCGCCTCGTTCCGGCTAAGTAACATGG | |
| AGCAGGTCGCGGATTTCGACACAATTTATCAGGCGATGATACAAATCTCCGTTGTACTTTGTTTCGCGCTTGGTATA | |
| ATCGCTGGGGGTCAAAGATGAGTGTTTTAGTGTATTCTTTCGCCTCTTTCGTTTTAGGTTGGTGCCTTCGTAGTGGC | |
| ATTACGTATTTTACCCGTTTAATGGAAACTTCCTCATGAAAAAGTCTTTAGTCCTCAAAGCCTCTGTAGCCGTTGCT | |
| ACCCTCGTTCCGATGCTGTCTTTCGCTGCTGAGGGTGACGATCCCGCAAAAGCGGCCTTTAACTCCCTGCAAGCCTC | |
| AGCGACCGAATATATCGGTTATGCGTGGGCGATGGTTGTTGTCATTGTCGGCGCAACTATCGGTATCAAGCTGTTTA | |
| AGAAATTCACCTCGAAAGCAAGCTGATAAACCGATACAATTAAAGGCTCCTTTTGGAGCCTTTTTTTTTGGAGATTT | |
| TCAACGTGAAAAAATTATTATTCGCAATTCCTTTAGTTGTTCCTTTCTATTCTCACAGTGCACAATCACATCTAGAC | |
| GCGGCCGCTCATCACCACCATCATCACTCTGCTGAACAAAAACTCATCTCAGAAGAGGATCTGAATGGTGCCGCACA | |
| AGCGAGCTCTGCTGAAACTGTTGAAAGTTGTTTAGCAAAATCCCATACAGAAAATTCATTTACTAACGTCTGGAAAG | |
| ACGACAAAACTTTAGATCGTTACGCTAACTATGAGGGCTGTCTGTGGAATGCTACAGGCGTTGTAGTTTGTACTGGT | |
| GACGAAACTCAGTGTTACGGTACATGGGTTCCTATTGGGCTTGCTATCCCTGAAAATGAGGGTGGTGGCTCTGAGGG | |
| TGGCGGTTCTGAGGGTGGCGGTTCTGAGGGTGGCGGTACTAAACCTCCTGAGTACGGTGATACACCTATTCCGGGCT | |
| ATACTTATATCAACCCTCTCGACGGCACTTATCCGCCTGGTACTGAGCAAAACCCCGCTAATCCTAATCCTTCTCTT | |
| GAGGAGTCTCAGCCTCTTAATACTTTCATGTTTCAGAATAATAGGTTCCGAAATAGGCAGGGGGCATTAACTGTTTA | |
| TACGGGCACTGTTACTCAAGGCACTGACCCCGTTAAAACTTATTACCAGTACACTCCTGTATCATCAAAAGCCATGT | |
| ATGACGCTTACTGGAACGGTAAATTCAGAGACTGCGCTTTCCATTCTGGCTTTAATGAGGATTTATTTGTTTGTGAA | |
| TATCAAGGCCAATCGTCTGACCTGCCTCAACCTCCTGTCAATGCTGGCGGCGGCTCTGGTGGTGGTTCTGGTGGCGG | |
| CTCTGAGGGTGGTGGCTCTGAGGGAGGCGGTTCCGGTGGTGGCTCTGGTTCCGGTGATTTTGATTATGAAAAGATGG | |
| CAAACGCTAATAAGGGGGCTATGACCGAAAATGCCGATGAAAACGCGCTACAGTCTGACGCTAAAGGCAAACTTGAT | |
| TCTGTCGCTACTGATTACGGTGCTGCTATCGATGGTTTCATTGGTGACGTTTCCGGCCTTGCTAATGGTAATGGTGC | |
| TACTGGTGATTTTGCTGGCTCTAATTCCCAAATGGCTCAAGTCGGTGACGGTGATAATTCACCTTTAATGAATAATT | |
| TCCGTCAATATTTACCTTCCCTCCCTCAATCGGTTGAATGTCGCCCTTTTGTCTTTGGCGCTGGTAAACCATATGAA | |
| TTTTCTATTGATTGTGACAAAATAAACTTATTCCGTGGTGTCTTTGCGTTTCTTTTATATGTTGCCACCTTTATGTA | |
| TGTATTTTCTACGTTTGCTAACATACTGCGTAATAAGGAGTCTTAATCATGCCAGTTCTTTTGGGTATTCCGTTATT | |
| ATTGCGTTTCCTCGGTTTCCTTCTGGTAACTTTGTTCGGCTATCTGCTTACTTTTCTTAAAAAGGGCTTCGGTAAGA | |
| TAGCTATTGCTATTTCATTGTTTCTTGCTCTTATTATTGGGCTTAACTCAATTCTTGTGGGTTATCTCTCTGATATT | |
| AGCGCTCAATTACCCTCTGACTTTGTTCAGGGTGTTCAGTTAATTCTCCCGTCTAATGCGCTTCCCTGTTTTTATGT | |
| TATTCTCTCTGTAAAGGCTGCTATTTTCATTTTTGACGTTAAACAAAAAATCGTTTCTTATTTGGATTGGGATAAAT | |
| AATATGGCTGTTTATTTTGTAACTGGCAAATTAGGCTCTGGAAAGACGCTCGTTAGCGTTGGTAAGATTCAGGATAA | |
| AATTGTAGCTGGGTGCAAAATAGCAACTAATCTTGATTTAAGGCTTCAAAACCTCCCGCAAGTCGGGAGGTTCGCTA | |
| AAACGCCTCGCGTTCTTAGAATACCGGATAAGCCTTCTATATCTGATTTGCTTGCTATTGGGCGCGGTAATGATTCC | |
| TACGATGAAAATAAAAACGGCTTGCTTGTTCTCGATGAGTGCGGTACTTGGTTTAATACCCGTTCTTGGAATGATAA | |
| GGAAAGACAGCCGATTATTGATTGGTTTCTACATGCTCGTAAATTAGGATGGGATATTATTTTTCTTGTTCAGGACT | |
| TATCTATTGTTGATAAACAGGCGCGTTCTGCATTAGCTGAACATGTTGTTTATTGTCGTCGTCTGGACAGAATTACT | |
| TTACCTTTTGTCGGTACTTTATATTCTCTTATTACTGGCTCGAAAATGCCTCTGCCTAAATTACATGTTGGCGTTGT | |
| TAAATATGGCGATTCTCAATTAAGCCCTACTGTTGAGCGTTGGCTTTATACTGGTAAGAATTTGTATAACGCATATG | |
| ATACTAAACAGGCTTTTTCTAGTAATTATGATTCCGGTGTTTATTCTTATTTAACGCCTTATTTATCACACGGTCGG | |
| TATTTCAAACCATTAAATTTAGGTCAGAAGATGAAATTAACTAAAATATATTTGAAAAAGTTTTCTCGCGTTCTTTG | |
| TCTTGCGATTGGATTTGCATCAGCATTTACATATAGTTATATAACCCAACCTAAGCCGGAGGTTAAAAAGGTAGTCT | |
| CTCAGACCTATGATTTTGATAAATTCACTATTGACTCTTCTCAGCGTCTTAATCTAAGCTATCGCTATGTTTTCPAG | |
| GATTCTAAGGGAAAATTAA | |

TABLE 10
Nucleotide sequence of pRHO6(s)

| | |
|---|---|
| TTAATAGCGACGATTTACAGAAGCAAGGTTATTCACTCACATATATTGATTTATGTACTGTTTCCATTAAAAAAGGT | (SEQ ID NO:12) |
| AATTCAAATGAAATTGTTAAATGTAATTAATTTTGTTTTCTTGATGTTTGTTTCATCATCTTCTTTTGCTCAGGTAA | |
| TTGAAATGAATAATTCGCCTCTGCGCGATTTTGTAACTTGGTATTCAAAGCAATCAGGCGAATCCGTTATTGTTTCT | |
| CCCGATGTAAAAGGTACTGTTACTGTATATTCATCTGACGTTAAACCTGAAAATCTACGCAATTTCTTTATTTCTGT | |
| TTTACGTGCAAATAATTTTGATATGGTAGGTTCTAACCCTTCCATTATTCAGAAGTATAATCCAAACAATCAGGATT | |
| ATATTGATGAATTGCCATCATCTGATAATCAGGAATATGATGATAATTCCGCTCCTTCTGGTGGTTTCTTTGTTCCG | |
| CAAAATGATAATGTTACTCAAACTTTTAAAATTAATAACGTTCGGGCAAAGGATTTAATACGAGTTGTCGAATTGTT | |
| TGTAAAGTCTAATACTTCTAAATCCTCAAATGTATTATCTATTGACGGCTCTAATCTATTAGTTGTTAGTGCTCCTA | |
| AAGATATTTTAGATAACCTTCCTCAATTCCTTTCAACTGTTGATTTGCCAACTGACCAGATATTGATTGAGGGTTTG | |
| ATATTTGAGGTTCAGCAAGGTGATGCTTTAGATTTTTCATTTGCTGCTGGCTCTCAGCGTGGCACTGTTGCAGGCGG | |
| TGTTAATACTGACCGCCTCACCTCTGTTTTATCTTCTGCTGGTGGTTCGTTCGGTATTTTTAATGGCGATGTTTTAG | |
| GGCTATCAGTTCGCGCATTAAAGACTAATAGCCATTCAAAAATATTGTCTGTGCCACGTATTCTTACGCTTTCAGGT | |
| CAGAAGGGTTCTATCTCTGTTGGCCAGAATGTCCCTTTTATTACTGGTCGTGTGACTGGTGAATCTGCCAATGTAAA | |
| TAATCCATTTCAGACGATTGAGCGTCAAAATGTAGGTATTTCCATGAGCGTTTTTCCTGTTGCAATGGCTGGCGGTA | |
| ATATTGTTCTGGATATTACCAGCAAGGCCGATAGTTTGAGTTCTTCTACTCAGGCAAGTGATGTTATTACTAATCAA | |
| AGAAGTATTGCTACAACGGTTAATTTGCGTGATGGACAGACTCTTTTACTCGGTGGCCTCACTGATTATAAAAACAC | |
| TTCTCAGGATTCTGGCGTACCGTTCCTGTCTAAAATCCCTTTAATCGGCCTCCTGTTTAGCTCCCGCTCTGATTCTA | |
| ACGAGGAAAGCACGTTATACGTGCTCGTCAAAGCAACCATAGTACGCGCCCTGTAGCGGCGCATTAAGCGCGGCGGG | |
| TGTGGTGGTTACGCGCAGCGTGACCGCTACACTTGCCAGCGCCCTAGCGCCCGCTCCTTTCGCTTTCTTCCCTTCCT | |
| TTCTCGCCACGTTCGCCGGCTTTCCCCGTCAAGCTCTAAATCGGGGGCTCCCTTTAGGGTTCCGATTTAGTGCTTTA | |
| CGGCACCTCGACCCCAAAAAACTTGATTTGGGTGATGGTTCACGTAGTGGGCCATCGCCCTGATAGACGGTTTTTCG | |
| CCCTTTGACGTTGGAGTCCACGTTCTTTAATAGTGGACTCTTGTTCCAAACTGGAACAACACTCAACCCTATCTCGG | |
| GCTATTCTTTTGATTTATAAGGGATTTTGCCGATTTCGGAACCACCATCAAACAGGATTTTCGCCTGCTGGGGCAAA | |
| CCAGCGTGGACCGCTTGCTGCAACTCTCTCAGGGCCAGGCGGTGAAGGGCAATCAGCTGTTGCCCGTCTCACTGGTG | |
| AAAAGAAAAACCACCCTGGATCCAAGCTTGCAGGTGGCACTTTTCGGGGAAATGTGCGCGGAACCCCTATTTGTTTA | |
| TTTTTCTAAATACATTCAAATATGTATCCGCTCATGAGACAATAACCCTGATAAATGCTTCAATAATATTGAAAAAG | |
| GAAGAGTATGAGTATTCAACATTTCCGTGTCGCCCTTATTCCCTTTTTTGCGGCATTTTGCCTTCCTGTTTTTGCTC | |
| ACCCAGAAACGCTGGTGAAAGTAAAAGATGCTGAAGATCAGTTGGGCGCACTAGTGGGTTACATCGAACTGGATCTC | |
| AACAGCGGTAAGATCCTTGAGAGTTTTCGCCCCGAAGAACGTTTTCCAATGATGAGCACTTTTAAAGTTCTGCTATG | |
| TGGCGCGGTATTATCCCGTATTGACGCCGGGCAAGAGCAACTCGGTCGCCGCATACACTATTCTCAGAATGACTTGG | |
| TTGAGTACTCACCAGTCACAGAAAAGCATCTTACGGATGGCATGACAGTAAGAGAATTATGCAGTGCTGCCATAACC | |
| ATGAGTGATAACACTGCGGCCAACTTACTTCTGACAACGATCGGAGGACCGAAGGAGCTAACCGCTTTTTTGCACAA | |
| CATGGGGGATCATGTAACTCGCCTTGATCGTTGGGAACCGGAGCTGAATGAAGCCATACCAAACGACGAGCGTGACA | |
| CCACGATGCCTGTAGCAATGGCAACAACGTTGCGCAAACTATTAACTGGCGAACTACTTACTCTAGCTTCCCGGCAA | |
| CAATTAATAGACTGGATGGAGGCGGATAAAGTTGCAGGACCACTTCTGCGCTCGGCCCTTCCGGCTGGCTGGTTTAT | |
| TGCTGATAAATCTGGAGCCGGTGAGCGTGGGTCTCGCGGTATCATTGCAGCACTGGGGCCAGATGGTAAGCCCTCCC | |
| GTATCGTAGTTATCTACACGACGGGGAGTCAGGCAACTATGGATGAACGAAATAGACAGATCGCTGAGATAGGTGCC | |
| TCACTGATTAAGCATTGGTAACTGTCAGACCAAGTTTACTCATATATACTTTAGATTGATTTAAAACTTCATTTTTA | |
| ATTTAAAAGGATCTAGGTGAAGATCCTTTTTGATAATCTCATGACCAAAATCCCTTAACGTGAGTTTTCGTTCCACT | |
| GTACGTAAGACCCCCAAGCTTGTCGACCGCAACGCAATTAATGTGAGTTAGCTCACTCATTAGGCACCCCAGGCTTT | |
| ACACTTTATGCTTCCGGCTCGTATGTTGTGTGGAATTGTGAGCGGATAACAATTTCACCCATGCTTTGGACAGGAAA | |
| CAGCTATGAAAAAGCTTTTATTCGCTATCCCGTTAGTTGTACCGTTCTATTCTCACTCTGCCGAGACAGTCGAATCC | |
| TGCCTGGCCAAGTCTCACACTGAGAATAGTTTCACAAATGTGTGGAAGGATGATAAGACCCTTGATCGATATGCCAA | |
| TTACGAAGGCTGCTTATGGAATGCCACCGGCGTCGTTGTCTGCACGGGCGATGAGACACAATGCTATGGCACGTGGG | |
| TGCCGATAGGCTTAGCCATACCGGAGAACGAAGGCGGCGGTAGCGAAGGCGGTGGCAGCGAAGGCGGTGGATCCGAA | |
| GGAGGTGGAACCAAGCCGCCGGAATATGGCGACACTCCGATACCTGGTTACACCTACATTAATCCGTTAGATGGAAC | |
| CTACCCTCCGGGCACCGAACAGAATCCTGCCAACCCGAACCCAAGCTTAGAAGAAAGCCAACCGTTAAACACCTTTA | |
| TGTTCCAAAACAACCGTTTTAGGAACCGTCAAGGTGCTCTTACCGTGTACACTGGAACCGTCACCCAGGGTACCGAT | |
| CCTGTCAAGACCTACTATCAATATACCCCGGTCTCGAGTAAGGCTATGTACGATGCCTATTGGAATGGCAAGTTTCG | |
| TGATTGTGCCTTTCACAGCGGTTTCAACGAAGACCCTTTTGTCTGCGAGTACCAGGGTCAGAGTAGCGATTTACCGC | |
| AGCCACCGGTTAACGCGGGTGGTGGTAGCGGCGGAGGCAGCGGCGGTGGTAGCGAAGGCGGAGGTAGCGAAGGAGGT | |
| GGCAGCGGAGGCGGTAGCGGCAGTGGCGACTTCGACTACGAGAAAATGGCTAATGCCAACAAAGGCGCCATGACTGA | |
| GAACGCTGACGAGAATGCACTGCAAAGTGATGCCAAGGGTAAGTTAGACAGCGTCGCCACAGACTATGGTGCTGCCA | |
| TCGACGGCTTTATCGGCGATGTCAGTGGTCTGGCTAACGGCAACGGAGCCACCGGAGACTTCGCAGGTTCGAATTCT ~ | |
| CAGATGGCCCAGGTTGGAGATGGGGACAACAGTCCGCTTATGAACAACTTTAGACAGTACCTTCCGTCTCTTCCGCA | |
| GAGTGTCGAGTGCCGTCCATTCGTTTTCTCTGCCGGCAAGCCTTACGAGTTCAGCATCGACTGCGATAAGATCAATC | |
| TTTTCCGCGGCGTTTTCGCTTTCTTGCTATACGTCGCTACTTTCATGTACGTTTTCAGCACTTTCGCCAATATTTTA | |
| CGCAACAAAGAAAGCTAGTGATCTCCTAGGAAGCCCGCCTAATGAGCGGGCTTTTTTTTTCTGGTATGCATCCTGAG | |
| GCCGATACTGTCGTCGTCCCCTCAAACTGGCAGATGCACGGTTACGATGCGCCCATCTACACCAACGTGACCTATCC | |
| CATTACGGTCAATCCGCCGTTTGTTCCCACGGAGAATCCGACGGGTTGTTACTCGCTCACATTTAATGTTGATGAAA | |
| GCTGGCTACAGGAAGGCCAGACGCGAATTATTTTTGATGGCGTTCCTATTGGTTAAAAAATGAGCTGATTTAACAAA | |
| AATTTAATGCGAATTTTAACAAAATATTAACGTTTACAATTTAAATATTTGCTTATACAATCTTCCTGTTTTTGGGG | |
| CTTTTCTGATTATCAACCGGGGTACATATGATTGACATGCTAGTTTTACGATTACCGTTCATCGATTCTCTTGTTTG | |
| CTCCAGACTCTCAGGCAATGACCTGATAGCCTTTGTAGATCTCTCAAAAATAGCTACCCTCTCCGGCATGAATTTAT | |
| CAGCTAGAACGGTTGAATATCATATTGATGGTGATTTGACTGTCTCCGGCCTTTCTCACCCTTTTGAATCTTTACCT | |
| ACACATTACTCAGGCATTGCATTTAAAATATATGAGGGTTCTAAAAATTTTTATCCTTGCGTTGAAATAAAGGCTTC | |
| TCCCGCAAAAGTATTACAGGGTCATAATGTTTTTGGTACAACCGATTTAGCTTTATGCTCTGAGGCTTTATTGCTTA | |
| ATTTTGCTAATTCTTTGCCTTGCCTGTATGATTTATTGGATGTTAATGCTACTACTATTAGTAGAATTGATGCCACC | |
| TTTTCAGCTCGCGCCCCAAATGAAAATATAGCTAAACAGGTTATTGACCATTTGCGAAATGTATCTAATGGTCAAAC | |
| TAAATCTACTCGTTCGCAGAATTGGGAATCAACTGTTACATGGAATGAAACTTCCAGACACCGTACTTTAGTTGCAT | |
| ATTTAAAACATGTTGAGCTACAGCACCAGATTCAGCAATTAAGCTCTAAGCCATCCGCAAAAATGACCTCTTATCAA | |
| AAGGAGCAATTAAAGGTACTCTCTAATCCTGACCTGTTGGAGTTTGCTTCCGGTCTGGTTCGCTTTGAAGCTCGAAT | |
| TAAAACGCGATATTTGAAGTCTTTCGGGCTTCCTCTTAATCTTTTTGATGCAATCCGCTTTGCTTCTGACTATAATA | |
| GTCAGGGTAAAGACCTGATTTTTGATTTATGGTCATTCTCGTTTTCTGAACTGTTTAAAGCATTTGAGGGGGATTCA | |
| ATGAATATTTATGACGATTCCGCAGTATTGGACGCTATCCAGTCTAAACATTTTACTATTACCCCCTCTGGCAAAAC | |
| TTCTTTTGCAAAAGCCTCTCGCTATTTTGGTTTTTATCGTCGTCTGGTAAACGAGGGTTATGATAGTGTTGCTCTTA | |
| CTATGCCTCGTAATTCCTTTTGGCGTTATGTATCTGCATTAGTTGAATGTGGTATTCCTAAATCTCAACTGATGAAT | |
| CTTTCTACCTGTAATAATGTTGTTCCGTTAGTTCGTTTTATTAACGTAGATTTTTCTTCCCAACGTCCTGACTGGTA | |
| TAATGAGCCAGTTCTTAAAATCGCATAAGGTAATTCACAATGATTAAAGTTGAAATTAAACCATCTCAAGCCCAATT | |
| TACTACTCGTTCTGGTGTTTCTCGTCAGGGCAAGCCTTATTCACTGAATGAGCAGCTTTGTTACGTTGATTTGGGTA | |
| ATGAATATCCGGTTCTTGTCAAGATTACTCTTGATGAAGGTCAGCCAGCCTATGCGCCTGGTCTGTACACCGTTCAT | |
| CTGTCCTCTTTCAAAGTTGGTCAGTTCGGTTCCCTTATGATTGACCGTCTGCGCCTCGTTCCGGCTAAGTAACATGG | |
| AGCAGGTCGCGGATTTCGACACAATTTATCAGGCGATGATACAAATCTCCGTTGTACTTTGTTTCGCGCTTGGTATA | |
| ATCGCTGGGGGTCAAAGATGAGTGTTTTAGTGTATTCTTTCGCCTCTTTCGTTTTAGGTTGGTGCCTTCGTAGTGGC | |
| ATTACGTATTTTACCCGTTTAATGGAAACTTCCTCATGAAAAAGTCTTTAGTCCTCAAAGCCTCTGTAGCCGTTGCT | |
| ACCCTCGTTCCGATGCTGTCTTTCGCTGCTGAGGGTGACGATCCCGCAAAAGCGGCCTTTAACTCCCTGCAAGCCTC | |
| AGCGACCGAATATATCGGTTATGCGTGGGCGATGGTTGTTGTCATTGTCGGCGCAACTATCGGTATCAAGCTGTTTA | |
| AGAAATTCACCTCGAAAGCAAGCTGATAAACCGATACAATTAAAGGCTCCTTTTGGAGCCTTTTTTTTTGGAGATTT | |
| TCAACGTGAAAAAATTATTATTCGCAATTCCTTTAGTTGTTCCTTTCTATTCTCACAGTGCACAATCACATCTAGAC | |
| GCGGCCGCTCATCACCACCATCATCACTCTGCTGAACAAAAACTCATCTCAGAAGAGGATCTGAATGGTGCCGCACA | |
| AGCGAGCTCTGCTGAAACTGTTGAAAGTTGTTTAGCAAAATCCCATACAGAAAATTCATTTACTAACGTCTGGAAAG | |
| ACGACAAAACTTTAGATCGTTACGCTAACTATGAGGGCTGTCTGTGGAATGCTACAGGCGTTGTAGTTTGTACTGGT | |
| GACGAAACTCAGTGTTACGGTACATGGGTTCCTATTGGGCTTGCTATCCCTGAAAATGAGGGTGGTGGCTCTGAGGG | |
| TGGCGGTTCTGAGGGTGGCGGTTCTGAGGGTGGCGGTACTAAACCTCCTGAGTACGGTGATACACCTATTCCGGGCT | |
| ATACTTATATCAACCCTCTCGACGGCACTTATCCGCCTGGTACTGAGCAAAACCCCGCTAATCCTAATCCTTCTCTT | |
| GAGGAGTCTCAGCCTCTTAATACTTTCATGTTTCAGAATAATAGGTTCCGAAATAGGCAGGGGGCATTAACTGTTTA | |
| TACGGGCACTGTTACTCAAGGCACTGACCCCGTTAAAACTTATTACCAGTACACTCCTGTATCATCAAAAGCCATGT | |
| ATGACGCTTACTGGAACGGTAAATTCAGAGACTGCGCTTTCCATTCTGGCTTTAATGAGGATTTATTTGTTTGTGAA | |
| TATCAAGGCCAATCGTCTGACCTGCCTCAACCTCCTGTCAATGCTGGCGGCGGCTCTGGTGGTGGTTCTGGTGGCGG | |
| CTCTGAGGGTGGTGGCTCTGAGGGAGGCGGTTCCGGTGGTGGCTCTGGTTCCGGTGATTTTGATTATGAAAAGATGG | |
| CAAACGCTAATAAGGGGGCTATGACCGAAAATGCCGATGAAAACGCGCTACAGTCTGACGCTAAAGGCAAACTTGAT | |
| TCTGTCGCTACTGATTACGGTGCTGCTATCGATGGTTTCATTGGTGACGTTTCCGGCCTTGCTAATGGTAATGGTGC | |
| TACTGGTGATTTTGCTGGCTCTAATTCCCAAATGGCTCAAGTCGGTGACGGTGATAATTCACCTTTAATGAATAATT | |
| TCCGTCAATATTTACCTTCCCTCCCTCAATCGGTTGAATGTCGCCCTTTTGTCTTTGGCGCTGGTAAACCATATGAA | |
| TTTTCTATTGATTGTGACAAAATAAACTTATTCCGTGGTGTCTTTGCGTTTCTTTTATATGTTGCCACCTTTATGTA | |
| TGTATTTTCTACGTTTGCTAACATACTGCGTAATAAGGAGTCTTAATCATGCCAGTTCTTTTGGGTATTCCGTTATT | |
| ATTGCGTTTCCTCGGTTTCCTTCTGGTAACTTTGTTCGGCTATCTGCTTACTTTTCTTAAAAAGGGCTTCGGTAAGA | |
| TAGCTATTGCTATTTCATTGTTTCTTGCTCTTATTATTGGGCTTAACTCAATTCTTGTGGGTTATCTCTCTGATATT | |
| AGCGCTCAATTACCCTCTGACTTTGTTCAGGGTGTTCAGTTAATTCTCCCGTCTAATGCGCTTCCCTGTTTTTATGT | |
| TATTCTCTCTGTAAAGGCTGCTATTTTCATTTTTGACGTTAAACAAAAAATCGTTTCTTATTTGGATTGGGATAAAT | |
| AATATGGCTGTTTATTTTGTAACTGGCAAATTAGGCTCTGGAAAGACGCTCGTTAGCGTTGGTAAGATTCAGGATAA | |
| AATTGTAGCTGGGTGCAAAATAGCAACTAATCTTGATTTAAGGCTTCAAAACCTCCCGCAAGTCGGGAGGTTCGCTA | |
| AAACGCCTCGCGTTCTTAGAATACCGGATAAGCCTTCTATATCTGATTTGCTTGCTATTGGGCGCGGTAATGATTCC | |
| TACGATGAAAATAAAAACGGCTTGCTTGTTCTCGATGAGTGCGGTACTTGGTTTAATACCCGTTCTTGGAATGATAA | |
| GGAAAGACAGCCGATTATTGATTGGTTTCTACATGCTCGTAAATTAGGATGGGATATTATTTTTCTTGTTCAGGACT | |
| TATCTATTGTTGATAAACAGGCGCGTTCTGCATTAGCTGAACATGTTGTTTATTGTCGTCGTCTGGACAGAATTACT | |
| TTACCTTTTGTCGGTACTTTATATTCTCTTATTACTGGCTCGAAAATGCCTCTGCCTAAATTACATGTTGGCGTTGT | |
| TAAATATGGCGATTCTCAATTAAGCCCTACTGTTGAGCGTTGGCTTTATACTGGTAAGAATTTGTATAACGCATATG | |
| ATACTAAACAGGCTTTTTCTAGTAATTATGATTCCGGTGTTTATTCTTATTTAACGCCTTATTTATCACACGGTCGG | |
| TATTTCAAACCATTAAATTTAGGTCAGAAGATGAAATTAACTAAAATATATTTGAAAAAGTTTTCTCGCGTTCTTTG | |
| TCTTGCGATTGGATTTGCATCAGCATTTACATATAGTTATATAACCCAACCTAAGCCGGAGGTTAAAAAGGTAGTCT | |
| CTCAGACCTATGATTTTGATAAATTCACTATTGACTCTTCTCAGCGTCTTAATCTAAGCTATCGCTATGTTTTCAAG | |
| GATTCTAAGGGAAAATTAA | |

TABLE 11
Nucleotide sequence of pRH07

| | |
|---|---|
| AATTCTCAGATGGCCCAGGTTGGAGATGGGGACAACAGTCCGCTTATGAACAACTTTAGACAGTACCTTCCGTCTCT | (SEQ ID NO: 13) |
| TCCGCAGAGTGTCGAGTGCCGTCCATTCGTTTTCGGAGCCGGCAAGCCTTACGAGTTCAGCATCGACTGCGATAAGA | |
| TCAATCTTTTCCGCGGCGTTTTCGCTTTCTTGCTATACGTCGCTACTTTCATGTACGTTTTCAGCACTTTCGCCAAT | |
| ATTTTACGCAACAAAGAAAGCTAGTGATCTCCTAGGAAGCCCGCCTAATGAGCGGGCTTTTTTTTTCTGGTATGCAT | |
| CCTGAGGCCGATACTGTCGTCGTCCCCTCAAACTGGCAGATGCACGGTTACGATGCGCCCATCTACACCAACGTGAC | |
| CTATCCCATTACGGTCAATCCGCCGTTTGTTCCCACGGAGAATCCGACGGGTTGTTACTCGCTCACATTTAATGTTG | |
| ATGAAAGCTGGCTACAGGAAGGCCAGACGCGAATTATTTTTGATGGCGTTCCTATTGGTTAAAAAATGAGCTGATTT | |
| AACAAAAATTTAATGCGAATTTTAACAAAATATTAACGTTTACAATTTAAATATTTGCTTATACAATCTTCCTGTTT | |
| TTGGGGCTTTTCTGATTATCAACCGGGGTACATATGATTGACATGCTAGTTTTACGATTACCGTTCATCGATTCTCT | |
| TGTTTGCTCCAGACTCTCAGGCAATGACCTGATAGCCTTTGTAGATCTCTCAAAAATAGCTACCCTCTCCGGCATGA | |
| ATTTATCAGCTAGAACGGTTGAATATCATATTGATGGTGATTTGACTGTCTCCGGCCTTTCTCACCCTTTTGAATCT | |
| TTACCTACACATTACTCAGGCATTGCATTTAAAATATATGAGGGTTCTAAAAATTTTTATCCTTGCGTTGAAATAAA | |
| GGCTTCTCCCGCAAAAGTATTACAGGGTCATAATGTTTTTGGTACAACCGATTTAGCTTTATGCTCTGAGGCTTTAT | |
| TGCTTAATTTTGCTAATTCTTTGCCTTGCCTGTATGATTTATTGGATGTTAATGCTACTACTATTAGTAGAATTGAT | |
| GCCACCTTTTCAGCTCGCGCCCCAAATGAAAATATAGCTAAACAGGTTATTGACCATTTGCGAAATGTATCTAATGG | |
| TCAAACTAAATCTACTCGTTCGCAGAATTGGGAATCAACTGTTACATGGAATGAAACTTCCAGACACCGTACTTTAG | |
| TTGCATATTTAAAACATGTTGAGCTACAGCACCAGATTCAGCAATTAAGCTCTAAGCCATCCGCAAAAATGACCTCT | |
| TATCAAAAGGAGCAATTAAAGGTACTCTCTAATCCTGACCTGTTGGAGTTTGCTTCCGGTCTGGTTCGCTTTGAAGC | |
| TCGAATTAAAACGCGATATTTGAAGTCTTTCGGGCTTCCTCTTAATCTTTTTGATGCAATCCGCTTTGCTTCTGACT | |
| ATAATAGTCAGGGTAAAGACCTGATTTTTGATTTATGGTCATTCTCGTTTTCTGAACTGTTTAAAGCATTTGAGGGG | |
| GATTCAATGAATATTTATGACGATTCCGCAGTATTGGACGCTATCCAGTCTAAACATTTTACTATTACCCCCTCTGG | |
| CAAAACTTCTTTTGCAAAAGCCTCTCGCTATTTTGGTTTTTATCGTCGTCTGGTAAACGAGGGTTATGATAGTGTTG | |
| CTCTTACTATGCCTCGTAATTCCTTTTGGCGTTATGTATCTGCATTAGTTGAATGTGGTATTCCTAAATCTCAACTG | |
| ATGAATCTTTCTACCTGTAATAATGTTGTTCCGTTAGTTCGTTTTATTAACGTAGATTTTTCTTCCCAACGTCCTGA | |
| CTGGTATAATGAGCCAGTTCTTAAAATCGCATAAGGTAATTCACAATGATTAAAGTTGAAATTAAACCATCTCAAGC | |
| CCAATTTACTACTCGTTCTGGTGTTTCTCGTCAGGGCAAGCCTTATTCACTGAATGAGCAGCTTTGTTACGTTGATT | |
| TGGGTAATGAATATCCGGTTCTTGTCAAGATTACTCTTGATGAAGGTCAGCCAGCCTATGCGCCTGGTCTGTACACC | |
| GTTCATCTGTCCTCTTTCAAAGTTGGTCAGTTCGGTTCCCTTATGATTGACCGTCTGCGCCTCGTTCCGGCTAAGTA | |
| ACATGGAGCAGGTCGCGGATTTCGACACAATTTATCAGGCGATGATACAAATCTCCGTTGTACTTTGTTTCGCGCTT | |
| GGTATAATCGCTGGGGGTCAAAGATGAGTGTTTTAGTGTATTCTTTCGCCTCTTTCGTTTTAGGTTGGTGCCTTCGT | |
| AGTGGCATTACGTATTTTACCCGTTTAATGGAAACTTCCTCATGAAAAAGTCTTTAGTCCTCAAAGCCTCTGTAGCC | |
| GTTGCTACCCTCGTTCCGATGCTGTCTTTCGCTGCTGAGGGTGACGATCCCGCAAAAGCGGCCTTTAACTCCCTGCA | |
| AGCCTCAGCGACCGAATATATCGGTTATGCGTGGGCGATGGTTGTTGTCATTGTCGGCGCAACTATCGGTATCAAGC | |
| TGTTTAAGAAATTCACCTCGAAAGCAAGCTGATAAACCGATACAATTAAAGGCTCCTTTTGGAGCCTTTTTTTTTGG | |
| AGATTTTCAACGTGAAAAAATTATTATTCGCAATTCCTTTAGTTGTTCCTTTCTATTCTCACAGTGCACAATCACAT | |
| CTAGACGCGGCCGCTCATCACCACCATCATCACTCTGCTGAACAAAAACTCATCTCAGAAGAGGATCTGAATGGTGC | |
| CGCACAAGCGAGCTCTGCTGAAACTGTTGAAAGTTGTTTAGCAAAATCCCATACAGAAAATTCATTTACTAACGTCT | |
| GGAAAGACGACAAAACTTTAGATCGTTACGCTAACTATGAGGGCTGTCTGTGGAATGCTACAGGCGTTGTAGTTTGT | |
| ACTGGTGACGAAACTCAGTGTTACGGTACATGGGTTCCTATTGGGCTTGCTATCCCTGAAAATGAGGGTGGTGGCTC | |
| TGAGGGTGGCGGTTCTGAGGGTGGCGGTTCTGAGGGTGGCGGTACTAAACCTCCTGAGTACGGTGATACACCTATTC | |
| CGGGCTATACTTATATCAACCCTCTCGACGGCACTTATCCGCCTGGTACTGAGCAAAACCCCGCTAATCCTAATCCT | |
| TCTCTTGAGGAGTCTCAGCCTCTTAATACTTTCATGTTTCAGAATAATAGGTTCCGAAATAGGCAGGGGGCATTAAC | |
| TGTTTATACGGGCACTGTTACTCAAGGCACTGACCCCGTTAAAACTTATTACCAGTACACTCCTGTATCATCAAAAG | |
| CCATGTATGACGCTTACTGGAACGGTAAATTCAGAGACTGCGCTTTCCATTCTGGCTTTAATGAGGATTTATTTGTT | |
| TGTGAATATCAAGGCCAATCGTCTGACCTGCCTCAACCTCCTGTCAATGCTGGCGGCGGCTCTGGTGGTGGTTCTGG | |
| TGGCGGCTCTGAGGGTGGTGGCTCTGAGGGAGGCGGTTCCGGTGGTGGCTCTGGTTCCGGTGATTTTGATTATGAAA | |
| AGATGGCAAACGCTAATAAGGGGGCTATGACCGAAAATGCCGATGAAAACGCGCTACAGTCTGACGCTAAAGGCAAA | |
| CTTGATTCTGTCGCTACTGATTACGGTGCTGCTATCGATGGTTTCATTGGTGACGTTTCCGGCCTTGCTAATGGTAA | |
| TGGTGCTACTGGTGATTTTGCTGGCTCTAATTCCCAAATGGCTCAAGTCGGTGACGGTGATAATTCACCTTTAATGA | |
| ATAATTTCCGTCAATATTTACCTTCCCTCCCTCAATCGGTTGAATGTCGCCCTTTTGTCTTTGGCGCTGGTAAACCA | |
| TATGAATTTTCTATTGATTGTGACAAAATAAACTTATTCCGTGGTGTCTTTGCGTTTCTTTTATATGTTGCCACCTT | |
| TATGTATGTATTTTCTACGTTTGCTAACATACTGCGTAATAAGGAGTCTTAATCATGCCAGTTCTTTTGGGTATTCC | |
| GTTATTATTGCGTTTCCTCGGTTTCCTTCTGGTAACTTTGTTCGGCTATCTGCTTACTTTTCTTAAAAAGGGCTTCG | |
| GTAAGATAGCTATTGCTATTTCATTGTTTCTTGCTCTTATTATTGGGCTTAACTCAATTCTTGTGGGTTATCTCTCT | |
| GATATTAGCGCTCAATTACCCTCTGACTTTGTTCAGGGTGTTCAGTTAATTCTCCCGTCTAATGCGCTTCCCTGTTT | |
| TTATGTTATTCTCTCTGTAAAGGCTGCTATTTTCATTTTTGACGTTAAACAAAAAATCGTTTCTTATTTGGATTGGG | |
| ATAAATAATATGGCTGTTTATTTTGTAACTGGCAAATTAGGCTCTGGAAAGACGCTCGTTAGCGTTGGTAAGATTCA | |
| GGATAAAATTGTAGCTGGGTGCAAAATAGCAACTAATCTTGATTTAAGGCTTCAAAACCTCCCGCAAGTCGGGAGGT | |
| TCGCTAAAACGCCTCGCGTTCTTAGAATACCGGATAAGCCTTCTATATCTGATTTGCTTGCTATTGGGCGCGGTAAT | |
| GATTCCTACGATGAAAATAAAAACGGCTTGCTTGTTCTCGATGAGTGCGGTACTTGGTTTAATACCCGTTCTTGGAA | |
| TGATAAGGAAAGACAGCCGATTATTGATTGGTTTCTACATGCTCGTAAATTAGGATGGGATATTATTTTTCTTGTTC | |
| AGGACTTATCTATTGTTGATAAACAGGCGCGTTCTGCATTAGCTGAACATGTTGTTTATTGTCGTCGTCTGGACAGA | |
| ATTACTTTACCTTTTGTCGGTACTTTATATTCTCTTATTACTGGCTCGAAAATGCCTCTGCCTAAATTACATGTTGG | |
| CGTTGTTAAATATGGCGATTCTCAATTAAGCCCTACTGTTGAGCGTTGGCTTTATACTGGTAAGAATTTGTATAACG | |
| CATATGATACTAAACAGGCTTTTTCTAGTAATTATGATTCCGGTGTTTATTCTTATTTAACGCCTTATTTATCACAC | |
| GGTCGGTATTTCAAACCATTAAATTTAGGTCAGAAGATGAAATTAACTAAAATATATTTGAAAAAGTTTTCTCGCGT | |
| TCTTTGTCTTGCGATTGGATTTGCATCAGCATTTACATATAGTTATATAACCCAACCTAAGCCGGAGGTTAAAAAGG | |
| TAGTCTCTCAGACCTATGATTTTGATAAATTCACTATTGACTCTTCTCAGCGTCTTAATCTAAGCTATCGCTATGTT | |
| TTCAAGGATTCTAAGGGAAAATTAATTAATAGCGACGATTTACAGAAGCAAGGTTATTCACTCACATATATTGATTT | |
| ATGTACTGTTTCCATTAAAAAAGGTAATTCAAATGAAATTGTTAAATGTAATTAATTTTGTTTTCTTGATGTTTGTT | |
| TCATCATCTTCTTTTGCTCAGGTAATTGAAATGAATAATTCGCCTCTGCGCGATTTTGTAACTTGGTATTCAAAGCA | |
| ATCAGGCGAATCCGTTATTGTTTCTCCCGATGTAAAAGGTACTGTTACTGTATATTCATCTGACGTTAAACCTGAAA | |
| ATCTACGCAATTTCTTTATTTCTGTTTTACGTGCAAATAATTTTGATATGGTAGGTTCTAACCCTTCCATTATTCAG | |
| AAGTATAATCCAAACAATCAGGATTATATTGATGAATTGCCATCATCTGATAATCAGGAATATGATGATAATTCCGC | |
| TCCTTCTGGTGGTTTCTTTGTTCCGCAAAATGATAATGTTACTCAAACTTTTAAAATTAATAACGTTCGGGCAAAGG | |
| ATTTAATACGAGTTGTCGAATTGTTTGTAAAGTCTAATACTTCTAAATCCTCAAATGTATTATCTATTGACGGCTCT | |
| AATCTATTAGTTGTTAGTGCTCCTAAGATATTTTAGATAACCTTCCTCAATTCCTTTCAACTGTTGATTTGCCAAC | |
| TGACCAGATATTGATTGAGGGTTTGATATTTGAGGTTCAGCAAGGTGATGCTTTAGATTTTTCATTTGCTGCTGGCT | |
| CTCAGCGTGGCACTGTTGCAGGCGGTGTTAATACTGACCGCCTCACCTCTGTTTTATCTTCTGCTGGTGGTTCGTTC | |
| GGTATTTTTAATGGCGATGTTTTAGGGCTATCAGTTCGCGCATTAAAGACTAATAGCCATTCAAAAATATTGTCTGT | |
| GCCACGTATTCTTACGCTTTCAGGTCAGAAGGGTTCTATCTCTGTTGGCCAGAATGTCCCTTTTATTACTGGTCGTG | |
| TGACTGGTGAATCTGCCAATGTAAATAATCCATTTCAGACGATTGAGCGTCAAAATGTAGGTATTTCCATGAGCGTT | |
| TTTCCTGTTGCAATGGCTGGCGGTAATATTGTTCTGGATATTACCAGCAAGGCCGATAGTTTGAGTTCTTCTACTCA | |
| GGCAAGTGATGTTATTACTAATCAAAGAAGTATTGCTACAACGGTTAATTTGCGTGATGGACAGACTCTTTTACTCG | |
| GTGGCCTCACTGATTATAAAAACACTTCTCAGGATTCTGGCGTACCGTTCCTGTCTAAAATCCCTTTAATCGGCCTC | |
| CTGTTTAGCTCCCGCTCTGATTCTAACGAGGAAAGCACGTTATACGTGCTCGTCAAAGCAACCATAGTACGCGCCCT | |
| GTAGCGGCGCATTAAGCGCGGCGGGTGTGGTGGTTACGCGCAGCGTGACCGCTACACTTGCCAGCGCCCTAGCGCCC | |
| GCTCCTTTCGCTTTCTTCCCTTCCTTTCTCGCCACGTTCGCCGGCTTTCCCCGTCAAGCTCTAAATCGGGGGCTCCC | |
| TTTAGGGTTCCGATTTAGTGCTTTACGGCACCTCGACCCCAAAAAACTTGATTTGGGTGATGGTTCACGTAGTGGGC | |
| CATCGCCCTGATAGACGGTTTTTCGCCCTTTGACGTTGGAGTCCACGTTCTTTAATAGTGGACTCTTGTTCCAAACT | |
| GGAACAACACTCAACCCTATCTCGGGCTATTCTTTTGATTTATAAGGGATTTTGCCGATTTCGGAACCACCATCAAA | |
| CAGGATTTTCGCCTGCTGGGGCAAACCAGCGTGGACCGCTTGCTGCAACTCTCTCAGGGCCAGGCGGTGAAGGGCAA | |
| TCAGCTGTTGCCCGTCTCACTGGTGAAAAGAAAAACCACCCTGGATCCAAGCTTGCAGGTGGCACTTTTCGGGGAAA | |
| TGTGCGCGGAACCCCTATTTGTTTATTTTTCTAAATACATTCAAATATGTATCCGCTCATGAGACAATAACCCTGAT | |
| AAATGCTTCAATAATATTGAAAAAGGAAGAGTATGAGTATTCAACATTTCCGTGTCGCCCTTATTCCCTTTTTTGCG | |
| GCATTTTGCCTTCCTGTTTTTGCTCACCCAGAAACGCTGGTGAAAGTAAAAGATGCTGAAGATCAGTTGGGCGCACT | |
| AGTGGGTTACATCGAACTGGATCTCAACAGCGGTAAGATCCTTGAGAGTTTTCGCCCCGAAGAACGTTTTCCAATGA | |
| TGAGCACTTTTAAAGTTCTGCTATGTGGCGCGGTATTATCCCGTATTGACGCCGGGCAAGAGCAACTCGGTCGCCGC | |
| ATACACTATTCTCAGAATGACTTGGTTGAGTACTCACCAGTCACAGAAAAGCATCTTACGGATGGCATGACAGTAAG | |
| AGAATTATGCAGTGCTGCCATAACCATGAGTGATAACACTGCGGCCAACTTACTTCTGACAACGATCGGAGGACCGA | |
| AGGAGCTAACCGCTTTTTTGCACAACATGGGGGATCATGTAACTCGCCTTGATCGTTGGGAACCGGAGCTGAATGAA | |
| GCCATACCAAACGACGAGCGTGACACCACGATGCCTGTAGCAATGGCAACAACGTTGCGCAAACTATTAACTGGCGA | |
| ACTACTTACTCTAGCTTCCCGGCAACAATTAATAGACTGGATGGAGGCGGATAAAGTTGCAGGACCACTTCTGCGCT | |
| CGGCCCTTCCGGCTGGCTGGTTTATTGCTGATAAATCTGGAGCCGGTGAGCGTGGGTCTCGCGGTATCATTGCAGCA | |
| CTGGGGCCAGATGGTAAGCCCTCCCGTATCGTAGTTATCTACACGACGGGGAGTCAGGCAACTATGGATGAACGAAA | |
| TAGACAGATCGCTGAGATAGGTGCCTCACTGATTAAGCATTGGTAACTGTCAGACCAAGTTTACTCATATATACTTT | |
| AGATTGATTTAAAACTTCATTTTTAATTTAAAAGGATCTAGGTGAAGATCCTTTTTGATAATCTCATGACCAAAATC | |
| CCTTAACGTGAGTTTTCGTTCCACTGTACGTAAGACCCCCAAGCTTGTCGACAGTGATAGACTAGTTAGACGCGTGC | |
| TTAAAGGCCTCCAATCCTCTTGGCGCGCCAATTCTATTTCAAGGAGACAGTCATAATGAAATACCTATTGCCTACGG | |
| CAGCCGCTGGATTGTTATTACTCGCGGCCCAGCCGGCCCTCTGATAAGATATCACTTGTTTAAACTCTGCTTGGCCC | |
| TCTTGGCCTTCTAGTAGACTTG | |

TABLE 12
Comparison of RH06-S and pRH05 Fab Display

| | DISPLAY | FITC | Background |
|---|---|---|---|
| pRHO6(s) E9 IPTG | 1.551 | 0.33 | 0.037 |
| pRHO6(s) E9 amp | 1.91 | 0.6 | 0.052 |
| pRHO6(s) E9 amp glu | 2.001 | 1.644 | 0.037 |
| pRHO5 E9 IPTG | 0.191 | 0.054 | 0.033 |
| pREO5 E9 glu | 0.88 | 0.299 | 0.037 |
| phagemid library | 0.667 | 0.052 | 0.035 |

A number of embodiments of the invention have been described. Nevertheless, it will be understood that various modifications may be made without departing from the spirit and scope of the invention. Accordingly, other embodiments are within the scope of the following claims.

What is claimed is:

1. A method of producing phage particles, the method comprising:
providing a set of host cells, wherein each of the host cells of the set comprises
a) a first expression unit comprising
(1) a first open reading frame, encoding a first polypeptide comprising
(i) an amino acid sequence to be displayed on a phage and
(ii) a portion of a phage coat protein of a filamentous phage, wherein the portion of the phage coat protein physically associates with phage particles, and
(2) a first promoter operably linked to the first open reading frame, and
b) a second expression unit comprising:
(1′) a second open reading frame, encoding a second polypeptide comprising a portion of the phage coat protein, and
(2′) a second promoter operably linked to the second open reading frame, wherein the second promoter is regulatable; and
maintaining the set of host cells under a first condition, wherein phage particles that include amino acid sequences to be displayed are produced.

2. The method of claim 1, wherein the amino acid sequence to be displayed varies among cells of the first set.

3. The method of claim 2, wherein the second polypeptide is invariant for all host cells of the set.

4. The method of claim 1, wherein the second polypeptide does not include a non-phage sequence of greater than five amino acids in length.

5. The method of claim 1, wherein the first condition increases activity of the regulatable promoter relative to a reference condition, and the phage particles produced by the first set of host cells are characterized by a first average number of copies of the first polypeptide.

6. The method of claim 1, wherein the first condition decreases activity of the regulatable promoter relative to a reference condition, and the phage particles produced by the first set of host cells are characterized by a first average number of copies of the first polypeptide.

7. The method of claim 1, wherein the first expression unit is a component of a nucleic acid element that further comprises a phage origin of replication and a phage packaging signal.

8. The method of claim 1, wherein the first polypeptide comprises an immunoglobulin variable domain sequence.

9. The method of claim 8, wherein the first expression unit further comprises an additional open reading frame that encodes a polypeptide comprising an immunoglobulin variable domain sequence, compatible with the immunoglobulin variable domain sequence in the first polypeptide.

10. The method of claim 1, wherein the second polypeptide comprises a mature full-length coat protein.

11. The method of claim 1, wherein the portion of the coat protein in the first and second open reading frame is a portion of a gene III protein.

12. The method of claim 11, wherein the gene III protein is a wild-type gene III protein.

13. The method of claim 11, wherein the gene III protein is a mutant of gene III protein that physically associates with phage particles less efficiently than wild-type.

14. The method of claim 1, wherein the portion of the coat protein in the first or second open reading frame is encoded by at least one synthetic codon.

15. The method of claim 1, wherein activity of the second promoter is regulated by an agent, and the first condition includes presence of the agent.

16. The method of claim 15, wherein the second promoter regulatable by the lacI repressor.

17. The method of claim 1, wherein the first promoter is a phage promoter.

18. The method of claim 17, wherein the phage promoter is a promoter naturally associated with an open reading frame encoding phage coat protein.

19. The method of claim 1, further comprising:
selecting a subset of the phage particles produced by the host cells,
introducing nucleic acid from phage particles of the subset into a second set of bacterial host cells,
maintaining at least two host cells of the second set under a second condition that results in a different level of activity of the regulatable, second promoter than the first condition, wherein phage particles produced by the second set of host cells are characterized by a second average number of copies of the first polypeptide physically attached to the phage, wherein the second average number of copies is different from the first average number of copies.

20. The method of claim 19, wherein the second average number of copies is less than the first average number of copies.

21. The method of claim 19, wherein the selecting comprises contacting phage to a target, and separating phage that bind the target from phage that do not bind the target.

22. The method of claim 19, further comprising selecting a subset of the phage particles produced by host cells of the second set.

23. A host cell comprising:
a) a first expression unit comprising (1) a first open reading frame and (2) a first promoter operably linked to the first open reading frame, wherein the first open reading frame encodes a first polypeptide comprising (i) an amino acid sequence to be displayed on a phage and (ii) a portion of a phage coat protein, the portion of the phage coat protein being capable of physically associating with phage particles, and
b) a second expression unit comprising (1′) a second open reading frame and (2′) a second promoter that is regulatable and operably linked to the second open reading frame, wherein the second open reading frame encodes a second polypeptide comprising a portion of the phage coat protein, the portion of the phage coat protein being capable of physically associating with phage particles.

24. The host cell of claim 23, wherein the first expression unit is a component of a nucleic acid element that further comprises a phage origin of replication and a phage packaging signal.

25. The host cell of claim 23, wherein the first expression unit and the second expression unit are on separate nucleic acid molecules.

26. A nucleic acid comprising:
a) a first expression unit comprising (1) an open reading frame and (2) a first promoter operably linked to the open reading frame, wherein the open reading frame encodes a first polypeptide comprising (i) an amino acid sequence to be displayed and (ii) a portion of a phage coat protein, the portion of the phage coat protein being capable of physically associating with phage particles, and
b) a second expression unit comprising a (1′) second open reading frame and (2′) a second promoter that is regulatable and operably linked to the second open reading frame, wherein the second open reading frame encodes a second polypeptide comprising a portion of the phage coat protein, the portion of the phage coat protein being capable of physically associating with phage particles.

27. The nucleic acid of claim 26, wherein the first promoter is a phage promoter and the second promoter is a lac promoter.

28. A phage genome that comprises the nucleic acid of claim 26.

29. A plurality of phage particles produced by the method of claim 1.

30. A library of host cells, the library comprising a plurality of host cells, each cell being according to claim 23, wherein the amino acid sequence to be displayed varies among cells of the plurality, and the host cells of the plurality collectively encode between 10³ to 10¹¹ different amino acid sequences to be displayed.

31. A library of phage particles, the library comprising a plurality of phage particles that comprise a phage genome of claim 28, wherein the amino acid sequence to be displayed varies among phage particles of the plurality, and the phage particles of the plurality collectively encode between 10³ to 10¹¹ different amino acid sequences to be displayed.

32. A phagemid comprising:
a) an open reading frame that encodes a polypeptide comprising an amino acid sequence to be displayed and a portion of a phage coat protein, wherein the amino acid sequence to be displayed is a heterologous sequence,
b) a promoter, operably linked to the open reading frame, wherein the promoter is (i) a phage promoter or (ii) a promoter that has less than 50% of the activity of the lac promoter in Luria Broth at 37° C.,
c) a phage origin of replication, and
d) a phage packaging signal.

33. A kit comprising:
(a) the phagemid of claim 32 or a phage particle or cell that contains the phagemid; and
(b) an isolated nucleic acid that comprises a nucleic acid sequence that includes an open reading frame that encodes a polypeptide comprising a portion of a phage coat protein and a regulatable promoter, operably linked to the open reading frame, or a phage particle or cell containing the nucleic acid.

34. A phagemid comprising:
a display cassette configured to receive a sequence encoding an amino acid sequence to be displayed; and
a sequence encoding at least a portion of a phage coat protein; and
a promoter that is identical, or substantially identical to an endogenous phage promoter, or includes a sequence that hybridizes to a strand of an endogenous phage promoter, the promoter being operably linked to the display cassette such that a transcript can be produced that includes a sequence inserted into the display cassette and the sequence encoding at least a portion of the phage coat protein.

35. A phagemid comprising:
a coding sequence encoding a polypeptide that comprises a first amino acid sequence to be displayed and at least a portion of a phage coat protein; and
a promoter that is identical, or substantially identical to an endogenous phage promoter, or includes a sequence that hybridizes to a strand of an endogenous phage promoter, the promoter being operably linked to the coding sequence.

36. The phagemid of claim 35, further comprising a second coding sequence that encodes a second amino acid sequence to be displayed, wherein the second amino acid sequence is not attached to a portion of phage coat protein, but can associate with the first amino acid sequence.

37. A method of providing phage particles that display a heterologous amino acid sequence, the method comprising:
providing a host cell that includes the phagemid of claim 32, and a genome of a helper phage, the genome comprising a regulatable promoter operably linked to a sequence encoding a coat protein whose abundance in the cell modulates incorporation of the amino acid sequence to be displayed into phage particles; and
maintaining the host cell under conditions, whereby phage particles that package the phagemid are produced.

38. The method of claim 37 wherein the conditions are selected to alter activity of the regulatable promoter relative to a reference activity level of the regulatable promoter.
